(19) United States
(12) Patent Application Publication
(10) Pub. No.: US 2022/0290245 A1
(43) Pub. Date: Sep. 15, 2022

(54) CANCER DETECTION AND CLASSIFICATION

(71) Applicant: The United States of America, as represented by the Secretary, Department of Health and Human Servic, Bethesda, MD (US)

(72) Inventors: Laura L. Elnitski, Germantown, MD (US); Gennady Margolin, Rockville, MD (US)

(73) Assignee: The United States of America, as represented by the Secretary, Department of Health and Human Servic, Bethesda, MD (US)

(21) Appl. No.: 17/632,783

(22) PCT Filed: Sep. 11, 2020
(86) PCT No.: PCT/US2020/050522

§ 371 Date: Feb. 3, 2022

Related U.S. Application Data
(60) Provisional application No. 62/898,670, filed on Sep. 11, 2019.

(51) Int. Cl. C12Q 1/6886; G16B 20/20

(52) U.S. Cl. CPC C12Q 1/6886; G16B 20/20; C12Q 2600/154

(57) ABSTRACT

The present application provides methods for the detection and classification of cancer. In one aspect, the application provides a method for detecting the presence of cancer in a subject or identifying a biological sample as from a subject with cancer by detecting the methylation status of a panel of eight genomic DNA segments. In another aspect, the application provides a method for classifying a cancer type in a subject or classifying a biological sample as from a subject with a particular cancer type by detecting the methylation status of a panel of 39 genomic DNA segments.

### CROSS REFERENCE TO RELATED APPLICATIONS

This application claims priority to U.S. Provisional Application No. 62/898,670, filed Sep. 11, 2019, which is incorporated by reference in its entirety.

### ACKNOWLEDGMENT OF GOVERNMENT SUPPORT

This invention was made with government support under Project No. ZIA HG200323-16 awarded by the National Institutes of Health, National Human Genome Research Institute. The government has certain rights in the invention.

### FIELD OF THE DISCLOSURE

The present disclosure relates to methods and processes for the detection and classification of cancer.

### BACKGROUND

Effective methods of tumor detection and diagnosis are essential for improving cancer survival. Current recommendations for cancer screening in the United States include cervical, prostate, colon and skin cancers, whereas lung cancer requires a strong family history and a ten-year smoking practice. Diagnosis is typically made from a cadre of screening and diagnostic tools that may include physical examination, radiographic imaging, sputum cytology, blood tests, endoscopy, and/or biopsies.

For many other cancer types, there are no screening guidelines for patients without symptoms and many tumors are found in later stages after significant advancement of tumor growth. Examples include ovarian and pancreatic tumors, for which the 5-year survival rates are 25 and 7%, respectively when detected in late stage. Late stage detection carries a poor survival rate for colon and breast cancers as well, even though these cancers are highly treatable when diagnosed early, 5-year survival rates are 20-25% when detected late.

Blood-based biopsies are emerging as noninvasive diagnostic modality that could be used for early cancer detection. Healthy human blood plasma contains cell-free DNA (cfDNA) that under normal conditions is believed to be primarily derived from apoptosis of normal cells of the hematopoietic lineage. In the event of malignancy, the pool of cfDNA can have traces of circulating tumor DNA (ctDNA) that can be detected through tumor-specific somatic variations and tumor specific methylation patterns. Although promising, the breadth of inter- and intra-tumoral heterogeneity and complexity of human cancer and human biology has impeded blood-based cancer screening.

### SUMMARY

The present application provides methods for the detection and classification of cancer. In one aspect, the application provides a method for detecting the presence of cancer in a subject or identifying a biological sample as from a subject with cancer by detecting the methylation status of a panel of eight genomic DNA segments. In another aspect, the application provides a method for classifying a cancer type in a subject or classifying a biological sample as from a subject with a particular cancer type by detecting the methylation status of a panel of 39 genomic DNA segments. The ability to classify samples as tumor or normal, and identify the tissue of origin using a minimal panel of markers provides a precision diagnostic tool for non-invasive cancer screening, monitoring tumor burden, and inferring drug sensitivities.

Described herein is the surprising finding that the methylation state of cytosines within a panel of eight genomic segments can be used as a biomarker for diagnosis of the presence of cancer in a subject, and to identify a biological sample from a subject with cancer. The genomic segments in the cancer detection panel contain the following genomic positions according to a GRCh37/hg19 reference human genome: chr6:88876741, chr6:150286508, chr7:19157193; chr10:14816201, chr12:129822259, chr14:89628169, chr17:40333009, and chr17:46655394.

Thus, a method is provided comprising obtaining a plurality of sequence reads of a methylation sequencing assay covering genomic segments of a biological sample from a human subject. The genomic segments contain the following genomic positions: chr6:88876741, chr6:150286508, chr7:19157193; chr10:14816201, chr12:129822259, chr14:89628169, chr17:40333009, and chr17:46655394 according to a GRCh37/hg19 reference human genome. A methylation status of altered or normal is assigned to each of the genomic segments by comparing methylation of CpG sites of the sequence reads covering the respective genomic segments to a normal control. The biological sample is identified as from a subject with cancer if at least one of the genomic segments is assigned an altered methylation status, or the biological sample is identified as from a subject without cancer if none of the genomic segments are assigned an altered methylation status.

Also described herein is the surprising finding that the methylation state of cytosines within a panel of 39 genomic segments can be used as a biomarker for classification of a cancer type in a subject, and to identify a biological sample from a subject with particular cancer type. The genomic segments in the cancer classification panel contain the following genomic positions according to a GRCh37/hg19 reference human genome: chr8:102451058, chr19:16189360, chr2:114035619, chr10:5566908, chr16:678127, chr6:106958645, chr4:142054417, chr10:116064472, chr11:60619955, chr16:51184392, chr2:8724060, chr13:113424938, chr2:240270793, chr2:219256101, chr11:8284312, chr19:1827498, chr19:18335182, chr9:140683797, chr10:21788638, chr8:1895558, chr7:27196759, chr7:4801993, chr10:114591733, chr4:156588387, chr10:1120831, chr12:54427173, chr2:25600752, chr10:8097689, chr6:133562470, chr10:8097331, chr10:103603810, chr17:46655394, chr5:140306231, chr2:66665428, chr2:176994448, chr2:61372138, chr2:176994764, chr8:97506675, and chr17:46711341.

Thus, a method for classifying a type of cancer in a subject is provided comprising obtaining a plurality of sequence reads of a methylation sequencing assay covering genomic segments of a biological sample from a human subject with cancer. The genomic segments contain the following genomic positions: chr8:102451058, chr19:16189360, chr2:114035619, chr10:5566908, chr16:678127, chr6:106958645, chr4:142054417, chr10:116064472, chr11:60619955, chr16:51184392, chr2:8724060, chr13:113424938, chr2:240270793, chr2:219256101, chr11:8284312, chr19:1827498, chr19:18335182, chr9:140683797, chr10:21788638, chr8:1895558, chr7:27196759, chr7:4801993, chr10:114591733, chr4:156588387, chr10:1120831, chr12:54427173, chr2:25600752, chr10:8097689, chr6:133562470, chr10:8097331, chr10:103603810, chr17:46655394, chr5:140306231, chr2:66665428, chr2:176994448, chr2:61372138, chr2:176994764, chr8:97506675, and chr17:46711341 according to a GRCh37/hg19 reference human genome. A methylation status of altered or normal is assigned to each of the genomic segments by comparing methylation of CpG sites of the sequence reads covering the respective genomic segments to a normal control. The type of cancer in the subject is classified into one of a plurality of different cancer types by comparing the methylation status of the genomic segments of the biological sample to a cancer type control, wherein the caner type control is the methylation status of the genomic segments in the different cancer types.

The biological sample from the subject can be, for example, a whole blood, serum, plasma, buccal epithelium, saliva, urine, stools, or bronchial aspirates sample. In some embodiments, the biological sample is a plasma or serum sample comprising cell-free DNA.

In several embodiments, the disclosed methods can be used to detect or classify colon cancer, rectum cancer, stomach cancer, pancreatic cancer, bladder cancer, head-neck cancer, lung cancer, breast cancer, kidney cancer, cervical cancer, liver cancer, prostate cancer, or uterine cancer.

In additional embodiments, computer-implemented methods, computer systems, and computer readable media are provided.

The foregoing and other features and advantages of this disclosure will become more apparent from the following detailed description of several embodiments which proceeds with reference to the accompanying figures.

### BRIEF DESCRIPTION OF THE DRAWINGS

FIGS. 1A and 1B. Tumor type classification of tumor and reference samples. FIG. 1A: Performance for 13 TCGA datasets and a blood reference. Samples are given in rows and classification categories (or classes) are in columns. Percentages in each row add up to 100% (only values ≥5% shown). FIG. 1B: Correct type recovery percentages using different criteria. Best score equals the values along the diagonal in FIG. 1A.

FIGS. 2A and 2B. Tumor-normal calling results. FIG. 2A: Percentage of TCGA tumor samples called as tumor (T; red) or normal (N; blue). Peripheral blood reference samples are shown in the last column. FIG. 2B: Percentage of TCGA normal samples predicted as normal or tumor. Sample numbers are given in parentheses.

FIGS. 3A-3F. Performance of type classification (FIGS. 3A and 3D) and tumor-normal calling (FIGS. 3B, 3C, 3E, 3F) procedures on non-TCGA data. Note that kidney data for 9 out of 39 classification probes (FIG. 3A) and 3 out of 8 tumor-normal calling probes (FIG. 3C) were absent across all samples. Two of the three latter missing probes were specifically discriminative for KIRC.T and KIRP.T, thus explaining poor kidney tumor calling. It is noted that WGBS blood plasma data from healthy controls (32 samples) were used, in aggregate, to filter out some candidate probes for tumor-type classification, but not for T-N calling.

FIGS. 4A-4C. Performance of tumor type classification and tumor-normal calling procedures in bisulfite amplicon sequence data on 46 amplicons. (FIG. 4A) Classification of normal plasma (“ref”) and tumor samples (“tissue.T”). Correct type recovery percentages with different criteria. (FIG. 4B, 4C) Tumor-normal calling: (FIG. 4B) Percentage of normal plasma and tumor samples called as tumor or normal. (FIG. 4C) Percentage of normal samples (“tissue.N”) predicted as normal or tumor. Sample numbers are given in parentheses.

FIGS. 5A-5D. Illustration of methylation signal detection at the locus within 200 bases of probe cg01163404. (FIG. 5A) average CpG methylation based on all reads (FIGS. 5B, 5C) methylation signal based on fully methylated and unmethylated reads, where reads are weighted: two functional forms for weights were assessed: (i) number of CpGs in a corresponding read, N, raised to some power, r, (i.e., Nr), here r=2, and (ii) some base, b, raised to the power of number of CpGs (i.e., bN), here b=2. For average methylation, (FIG. 5A), cu and cm are counts of unmethylated and methylated CpGs in a locus, while for (FIGS. 5B, 5C) cu and cm are (weighted) counts of fully unmethylated and fully methylated reads, respectively. (FIG. 5D) Average number of all, fully unmethylated and fully methylated reads in each group of samples, and corresponding average number of CpGs per read. One can see that low numbers of CpGs, in addition to low coverage for dilute signal, hamper improved signal detection in WGBS.

FIG. 6. Schematic of data processing. Initially, select and binarize classification probes (NAs are in gray). Next, for each sample, obtain and binarize methylation values at classification probes. Finally, calculate the mean distances across classification probes from the sample values to each of the classification types/categories; rank candidate categories from best to worst suitable and analyze prediction performance

FIG. 7. Illustration of correct type recoveries using ranks of the candidate types, or alternatively, using types within certain range. Five candidate types considered here instead of 14 used in actual analysis.

FIG. 8A-8C. Analysis of classification performance using 39-marker panel on TCGA and blood reference data (see Table 1). (FIG. 8A) Distribution of ranks of correct type for the 14 datasets considered. (FIG. 8B) Distribution of the number of types within range (see Methods) for the 14 datasets considered. The red dots indicate the mean values. (FIG. 8C) The mean values from FIGS. 8A and 8B summarized.

FIG. 9. Analysis of classification performance using 39-marker panel on non-TCGA data (see Table 2). The mean values of ranks of correct type (blue) and the mean values of types within range (see Methods) for the datasets considered, grouped by origin.

FIG. 10. Type classification of the normal TCGA samples with 39 markers. Except for BLCA.N, samples overwhelmingly tend to be classified either with correct tissue type or as reference. Note that these samples were not used in the selection of the 39 markers.

FIG. 11 depicts an exemplary computing environment.

### SEQUENCE LISTING

The nucleic acid sequences listed in the accompanying sequence listing are shown using standard letter abbreviations for nucleotide bases as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “98800-02 Sequence Listing.txt” (˜12 kb), which was created on Sep. 11, 2020 which is incorporated by reference herein.

### DETAILED DESCRIPTION

Biomarkers with high specificity and sensitivity are needed for use in clinically applicable, non-invasive blood-based diagnostic testing. To this end, provided herein is the identification of genomic segments, the methylation of which can be used to robustly detect multiple cancer types and to effectively classify the tissue of origin.

A panel of eight different genomic segments is provided, the methylation of which can be used to robustly detect tumors of all types with a true positive rate (TPR) of greater than 90% and a false positive rate (FPR) of less than 0.04%, facilitating the use of this panel for non-invasive blood-based testing.

Further, a second panel of 39 different genomic segments is provided, the methylation of which can be used to classify the type of tumor with a TPR ranging from 98% to 69% depending on tumor type and sample. The multi-cancer and cancer-specific panels are computationally validated in independent data from colon, pancreas, lung, breast, kidney, liver, and prostate solid tumor datasets, with minimal decreases in performance.

The ability to classify samples as tumor or normal, and identify the tissue of origin using a minimal panel of markers provides a precision diagnostic tool for non-invasive cancer screening, monitoring tumor burden, and inferring drug sensitivities.

### I. Abbreviations

BLCA Bladder urothelial carcinoma
BRCA Breast invasive carcinoma
CRAD Colon adenocarcinoma and rectum adenocarcinoma
HNSC Head-neck squamous cell carcinoma
GEO Gene expression omnibus
KIRC Kidney renal clear cell carcinoma
KIRP Cervical kidney renal papillary cell carcinoma
LIHC Liver hepatocellular carcinoma
LUAD Lung adenocarcinoma
LUSC Lung squamous cell carcinoma
PAAD Pancreatic adenocarcinoma
PRAD Prostate adenocarcinoma
STAD Stomach adenocarcinoma
TCGA The cancer genome atlas
UCEC Uterine corpus endometrial carcinoma
WGBS Whole genome bisulfite sequencing

### II. Summary of Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

About: Plus or minus 5% from a set amount. For example, “about 5” refers to 4.75 to 5.25. A ratio of “about 5:1” refers to a ratio of from 4.75:1 to 5.25:1.

Amplicon: The nucleic acid products resulting from the amplification of a target nucleic acid sequence. Amplification is often performed by PCR. Amplicons can range in size from 20 base pairs to 15000 base pairs in the case of long range PCR, but are more commonly 100-1000 base pairs for bisulfite-treated DNA used for methylation analysis.

Amplification: To increase the number of copies of a nucleic acid molecule. The resulting amplification products are called “amplicons.” Amplification of a nucleic acid molecule (such as a DNA or RNA molecule) refers to use of a technique that increases the number of copies of a nucleic acid molecule in a sample. An example of amplification is the polymerase chain reaction (PCR), in which a sample is contacted with a pair of oligonucleotide primers under conditions that allow for the hybridization of the primers to a nucleic acid template in the sample. The product of amplification can be characterized by such techniques as electrophoresis, restriction endonuclease cleavage patterns, oligonucleotide hybridization or ligation, and/or nucleic acid sequencing. In some embodiments, the methods provided herein can include a step of producing an amplified nucleic acid under isothermal or thermal variable conditions.

As used herein the term “selectively,” when used in reference to “amplifying” (or grammatical equivalents), refers to preferentially amplifying a first nucleic acid in a sample compared to one or more other nucleic acids in the sample. The term can refer to producing one or more copies of the first nucleic acid and substantially no copies of the other nucleic acids. The term can also refer to producing a detectable amount of copies of the first nucleic acid and an undetectable (or insignificant) amount of copies of the other nucleic acids under a particular detection condition used.

Biological Sample: A sample obtained from a subject. As used herein, biological samples include all clinical samples containing genomic DNA (such as cell-free genomic DNA) useful for cancer diagnosis and classification, including, but not limited to, cells, tissues, and bodily fluids, such as: blood, derivatives and fractions of blood (such as serum or plasma), buccal epithelium, saliva, urine, stools, bronchial aspirates, sputum, biopsy (such as tumor biopsy), and CVS samples. A “biological sample” obtained or derived from a subject includes any such sample that has been processed in any suitable manner (for example, processed to isolate genomic DNA for bisulfite treatment) after being obtained from the subject.

Bisulfite treatment: The treatment of DNA with bisulfite or a salt thereof, such as sodium bisulfite (NaHSO₃). Bisulfite reacts readily with the 5,6-double bond of cytosine, but poorly with methylated cytosine. Cytosine reacts with the bisulfite ion to form a sulfonated cytosine reaction intermediate which is susceptible to deamination, giving rise to a sulfonated uracil. The sulfonate group can be removed under alkaline conditions, resulting in the formation of uracil. Uracil is recognized as a thymine by polymerases and amplification will result in an adenine-thymine base pair instead of a cytosine-guanine base pair.

Cancer: A cancer is a biological condition in which a malignant tumor or other neoplasm has undergone characteristic anaplasia with loss of differentiation, increased rate of growth, invasion of surrounding tissue, and which is capable of metastasis. A malignant cancer is a new and abnormal growth of tissue or cells in which the growth is uncontrolled and progressive. Non-limiting examples of types of cancer include lung cancer, stomach cancer, colon cancer, breast cancer, uterine cancer, bladder, head and neck, kidney, liver, ovarian, pancreas, prostate, and rectum cancer.

Features often associated with malignancy include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc.

In many instances, cancer is characterized as including the presence of a tumor in a subject. The amount of a tumor in a subject is the “tumor burden” which can be measured as the number, volume, or weight of the tumor. A tumor that does not metastasize is referred to as “benign.” A tumor that invades the surrounding tissue and/or can metastasize is referred to as “malignant.”

Examples of hematological cancers include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of cancers that can include a solid tumor, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma breast cancer or colon cancer.

In several embodiments, the disclosed methods can be used it identify a subject with a cancer including an established tumor, and optionally also classify the established tumor in the subject. An “established” or “existing” tumor is an existing tumor that can be discerned by diagnostic tests. In some embodiments, and established tumor can be palpated. In some embodiments, and “established tumor” is at least 500 mm³, such as at least 600 mm³, at least 700 mm³, or at least 800 mm³ in size. In other embodiments, the tumor is at least 1 cm long. With regard to a solid tumor, and established tumor generally has a robust blood supply, and has induced Tregs and myeloid derived suppressor cells (MDSC).

Cell-free DNA: DNA which is no longer fully contained within an intact cell, for example DNA found in plasma or serum.

Consists of or consists essentially of: With regard to a polynucleotide (such as primers, a target nucleic acid molecule, or an amplicon), a polynucleotide consists essentially of a specified nucleotide sequence if it does not include any additional nucleotides. However, the polynucleotide can include additional non-nucleic acid components, such as labels (for example, fluorescent, radioactive, or solid particle labels), sugars or lipids. With regard to a polynucleotide, a polynucleotide that consists of a specified nucleotide sequence does not include any additional nucleotides, nor does it include additional non-nucleic acid components, such as lipids, sugars or labels.

Control: A sample or standard used for comparison with an experimental sample. In some embodiments, the control is a sample obtained from a healthy subject (such as a subject without cancer) or a non-tumor tissue sample obtained from a patient diagnosed with cancer. In some embodiments, the control is a historical control or standard reference value or range of values (such as a previously tested control sample, such as a group of cancer patients with poor prognosis, or group of samples that represent baseline or normal values, such as the level of methylation of a target nucleic acid or particular CpG site in non-tumor tissue or a subject without cancer.

As used herein, a “normal” control is a sample or standard from or based on a subject without cancer or non-cancerous tissue from a subject.

CpG Site: A di-nucleotide DNA sequence comprising a cytosine followed by a guanine in the 5′ to 3′ direction. The cytosine nucleotides of CpG sites in genomic DNA are the target of intracellular methytransferases and can have a methylation status of methylated or not methylated. Reference to “methylated CpG site” or similar language refers to a CpG site in genomic DNA having a 5-methylcytosine nucleotide.

Detecting: To identify the existence, presence, or fact of something. General methods of detecting are known to the skilled artisan and may be supplemented with the protocols and reagents disclosed herein. Detecting can include determining if a particular nucleotide, for example a cytosine, guanine, or methylated cytosine, is present or absent in a sequence.

Diagnosis: The process of identifying a disease (such as cancer) by its signs, symptoms and results of various tests. In several embodiments a diagnosis of the presence of cancer in a subject (or an increased likelihood of the presence of the cancer in the subject or a particular type of cancer in a subject) can be made based on the methylation state of CpG within regions of genomic DNA from a sample from the subject as described herein. The conclusion reached through that process is also called “a diagnosis.” Forms of testing performed include blood tests, stool tests, medical imaging, urinalysis, endoscopy, biopsy, and epigenetic characterization of genomic DNA.

DNA (deoxyribonucleic acid): DNA is a long chain polymer which comprises the genetic material of most living organisms. The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, for instance, it is appropriate to generate probes or primers from the reverse complement sequence of the disclosed nucleic acid molecules.

Genomic segment: A contiguous sequence of genomic DNA no more than 2000 bases in length.

Label: A detectable molecule that is conjugated directly or indirectly to a second molecule, such as an oligonucleotide primer, to facilitate detection, purification, or analysis of the second molecule. The labels used herein for labeling nucleic acid molecules (such as oligonucleotide primers) are conventional. Specific, non-limiting examples of labels that can be used to label oligonucleotide primers include fluorophores and additional nucleotide sequences linked to the 5′end of the primer (for example, bar codes and adaptor sequences to facilitate sequencing reactions).

Methylation: The addition of a methyl group (—CH₃) to cytosine nucleotides of CpG sites in DNA. DNA methylation, the addition of a methyl group onto a nucleotide, is a post-replicative covalent modification of DNA that is catalyzed by a DNA methyltransferase enzyme. In biological systems, DNA methylation can serve as a mechanism for changing the structure of DNA without altering its coding function or its sequence.

Methylation sequencing assay: A sequencing assay that detects the methylation status of one or more CpG sites in DNA. A non-limiting example of a methylation sequencing assay is a sequencing assay performed on bisulfite-treated and amplified genomic DNA.

Methylation status: The status of methylation (methylated or not methylated) of the cytosine nucleotide of one or more CpG sites within a genomic sequence. An “altered” methylation status compared to a control (such as a normal control) is the opposite of the methylation state in the normal control. For example, if the normal control status of a particular CpG is “methylated,” then the altered methylation state of that CpG compared to the normal control would be “not methylated.”

Primers: Primers are nucleic acid molecules, usually DNA oligonucleotides of about 10-50 nucleotides in length (longer lengths are also possible). Typically, primers are at least about 15 nucleotides in length, such as at least about 20, 25, 30, or 40 nucleotides in length. For example, a primer can be about 10-50 nucleotides in length, such as, 10-30, 15-20, 15-25, 15-30, or 20-30 nucleotides in length. Primers can also be of a maximum length, for example no more than 25, 30, 40, or 50 nucleotides in length. Forward and reverse primers may be annealed to a complementary target DNA strand by nucleic acid hybridization to form hybrids between the primers and the target DNA strand, and then extended along the target DNA strand by a DNA polymerase enzyme to form an amplicon. One of skill in the art will appreciate that the hybridization specificity of a particular probe or primer typically increases with its length. Thus, for example, a probe or primer including 20 consecutive nucleotides typically will anneal to a target with a higher specificity than a corresponding probe or primer of only 15 nucleotides. In some embodiments, forward and reverse primers are used in combination in a bisulfite amplicon sequencing assay.

Sensitivity and specificity: Statistical measurements of the performance of a binary classification test. Sensitivity measures the proportion of actual positives which are correctly. Specificity measures the proportion of negatives which are correctly identified.

Sequence Read: A sequence (e.g., of about 300 bp) of contiguous base pairs of a nucleic acid molecule. The sequence read may be represented symbolically by the base pair sequence (in ATCG) of the sample portion. It may be stored in a memory device and processed as appropriate to determine whether it matches a reference sequence or meets other criteria. A sequence read may be obtained directly from a sequencing apparatus or indirectly from stored sequence information concerning a sample.

Subject: A living multi-cellular vertebrate organism, a category that includes human and non-human mammals.

Target nucleic acid molecule: A nucleic acid molecule whose detection, amplification, quantitation, qualitative detection, or a combination thereof, is intended. The nucleic acid molecule need not be in a purified form. Various other nucleic acid molecules can also be present with the target nucleic acid molecule. For example, the target nucleic acid molecule can be a specific nucleic acid molecule of which the amplification and/or evaluation of methylation status is intended. Purification or isolation of the target nucleic acid molecule, if needed, can be conducted by methods known to those in the art, such as by using a commercially available purification kit or the like.

### III. Detecting Cancer

The present disclosure relates to diagnosis of cancer in a subject using DNA methylation of specific segments of genomic DNA from the subject as a biomarker. Having identified the specified segments as a highly sensitive and specific cancer markers, methods of detecting cancer in a subject and/or a biological sample from the subject are provided.

As disclosed herein, the methylation state of cytosines within a panel of eight genomic segments can be used as a biomarker for diagnosis of the presence of cancer in a subject, and to identify a biological sample from a subject with cancer. The genomic segments in the panel contain the following genomic positions: chr6:88876741, chr6:150286508, chr7:19157193; chr10:14816201, chr12:129822259, chr14:89628169, chr17:40333009, and chr17:46655394 according to a GRCh37/hg19 reference human genome.

The cancer can be any type of cancer, including but not limited to colon cancer, rectal cancer, stomach cancer, pancreatic cancer, bladder cancer, head-neck cancer, lung cancer, breast cancer, kidney cancer, cervical cancer, liver cancer, uterine cancer, ovarian cancer, and prostate cancer.

Unless context indicated otherwise, reference to positions of genomic DNA herein refers to the corresponding nucleotides of the human genome version GRCh37/hg19. Unless context indicated otherwise, reference to a particular CpG site position refers the position of the cytosine nucleotide of the CpG site in the forward strand of the human genome version GRCh37/hg19. It should be noted that CpG sites are symmetric in the forward (+) and reverse (−) strands of DNA (as C pairs to G and G to C). Therefore, the methods and systems provided herein for analysis of the methylation status of CpG sites can be applied to either or both of the forward and reverse strands of the human genome. In the context of the reverse strand, the genome position of the cytosine of a CpG site is in an n+1 position. In some embodiments, the methylation status of CpG sites in the forward strand of particular genomic regions are analyzed according to the methods and systems provided herein. In some embodiments, the methylation status of CpG sites in the reverse strand of particular genomic regions are analyzed according to the methods and systems provided herein. In some embodiments, the methylation status of CpG sites in the forward and reverse strands of particular genomic regions are analyzed according to the methods and systems provided herein.

Detecting cancer in a subject can include obtaining a biological sample from the subject. The sample can be any sample that includes genomic DNA. Such samples include, but are not limited to, tissue from biopsies (including formalin-fixed paraffin-embedded tissue), autopsies, and pathology specimens; sections of tissues (such as frozen sections or paraffin-embedded sections taken for histological purposes); body fluids, such as blood, sputum, serum, ejaculate, or urine, or fractions of any of these; and so forth. In one particular example, the sample from the subject is a tissue biopsy sample. In another specific example, the sample from the subject is urine. In some embodiments the biological sample is a plasma or serum sample comprising cell-free DNA. In several embodiments, the biological sample is from a subject suspected of having a cancer, such as colon cancer, rectal cancer, stomach cancer, pancreatic cancer, bladder cancer, head-neck cancer, lung cancer, breast cancer, kidney cancer, cervical cancer, liver cancer, uterine cancer, ovarian cancer, and prostate cancer. In some embodiments, the biological sample is a tumor sample or a suspected tumor sample. For example, the sample can be a biopsy sample from at or near or just beyond the perceived leading edge of a tumor in a subject. Testing of the sample using the methods provided herein can be used to confirm the location of the leading edge of the tumor in the subject. This information can be used, for example, to determine if further surgical removal of tumor tissue is appropriate.

In some embodiments, an amplicon generated from cell-free DNA derived from blood (or a portion thereof, such as plasma or serum) can be used to detect the methylation of circulating tumor DNA (ctDNA). There are many studies detecting and assessing the fraction of ctDNA based on mutations. However, mutation-based detection is only specific to the tumors harboring those mutations and without a detailed understanding of normal samples it is not always clear what levels of ctDNA should be considered abnormal and warrant intervention. Conversely, the methylation state of cytosines within the disclosed genomic segments may be similar throughout different tumor types and may complement or supersede mutation markers for better diagnosis.

In some embodiments, a plurality of sequence reads of a methylation sequencing assay are obtained to detect the methylation of circulating tumor DNA (ctDNA). The sequence reads cover the panel of eight genomic segments as provided herein for diagnosis of the presence of cancer in a subject, and to identify a biological sample from a subject with cancer. Thus, the sequence reads cover a panel of eight genomic segments containing the following eight genomic positions: chr6:88876741, chr6:150286508, chr7:19157193; chr10:14816201, chr12:129822259, chr14:89628169, chr17:40333009, and chr17:46655394 according to a GRCh37/hg19 reference human genome. A methylation status of altered or normal is assigned to each of the genomic segments by comparing methylation of CpG sites of the sequence reads covering the respective genomic segments to a normal control. The biological sample is identified as from a subject with cancer if at least one (such as at least two, at least three, at least four, at least five, at least six, at least seven, or all eight) of the genomic segments is assigned an altered methylation status. Alternatively, the biological sample is identified as from a subject without cancer is none of the genomic segments are assigned an altered methylation status.

Each genomic segment contains an appropriate amount of contiguous DNA containing the chr6:88876741, chr6:150286508, chr7:19157193; chr10:14816201, chr12:129822259, chr14:89628169, chr17:40333009, or chr17:46655394 genomic position to capture a sufficient number of the CpG sites surrounding these positions to determine whether or not the genomic segment has an altered or normal methylation status. In some embodiments, each genomic segment independently contains plus or minus up to 300 bases (for example, up to 200 bases, up to 100 bases, or up to 50 bases) of the genomic positions, such as plus or minus 50 to 300 bases of the genomic positions. In some embodiments, the genomic segments containing chr6:88876741, chr6:150286508, chr7:19157193; chr10:14816201, chr12:129822259, chr14:89628169, chr17:40333009, and chr17:46655394 correspond to genomic sequence comprising or consisting of SEQ ID NOs: 25-32, respectively.

Any appropriate method can be used to assign a methylation status of altered or normal to the eight genomic segments. For example, in some embodiments, a genomic segment is assigned an altered methylation status if the CpG sites of the genomic segment are not methylated or have a low frequency of methylation (such as less than 20%) in non-cancerous (normal) tissue and the CpG sites of the genomic segment from the biological sample are identified as hypermethylated (such as more than 80% of the CpG sites in the genomic segment are methylated). In some embodiments, a genomic segment is assigned an altered methylation status if the CpG sites of the genomic segment are all methylated or have a high frequency of methylation (such as more than 80%) in non-cancerous (normal) tissue and the CpG sites of the genomic segment from the biological sample are identified as hypomethylated (such as less than 20% of the CpG sites in the genomic segment are methylated).

In some embodiments, assigning a methylation status to the genomic segments containing chr17:40333009, chr17:46655394, chr6:88876741, chr6:150286508, and chr7:19157193 comprises calculating a ratio X₁ according to: X₁=F₂/(F₁+F₂). F₁ is a frequency of sequence reads in the plurality of sequence reads corresponding to a particular genomic segment where less than 40% (such as less than 30%, less than 25%, less than 20%, less than 10%, or none) of the CpG sites are methylated based on the sequence read. F₂ is a frequency of sequence reads in the plurality corresponding to a particular genomic segment where at least 60% (such as at least 70%, at least 80%, at least 90%, or all) of the CpG sites are methylated based on the sequence read. The ratio X₁ calculated for the sequence reads of genomic segments of the biological sample is compared to a normal control (such as a corresponding normal control ratio X₁ based on genomic segments from non-cancerous tissue). A genomic segment is assigned an altered methylation status if there is an increase in the ratio X₁ compared to the normal control (such as an increase of at least 50% or at least 100%, or at least one standard deviation, or at least two standard deviations compared to the normal control) and a genomic segment is assigned a normal methylation status if there is not an increase in the ratio X₁ compared to the normal control (such as an increase of at least 50% or at least 100%, or at least one standard deviation, or at least two standard deviations compared to the normal control).

In some embodiments, assigning a methylation status to the genomic segments containing chr10:14816201, chr12:129822259, and chr14:89628169 comprises calculating a ratio X₂ according to: X₂=F₁/(F₁+F₂). F₁ is a frequency of sequence reads in the plurality of sequence reads corresponding to a particular genomic segment where less than 40% (such as less than 30%, less than 20%, less than 10%, or none) of the CpG sites are methylated based on the sequence read. F₂ is a frequency of sequence reads in the plurality corresponding to a particular genomic segment where at least 60% (such as at least 70%, at least 80% at least 90%, or all) of the CpG sites are methylated based on the sequence read. The ratio X₂ calculated for the sequence reads of genomic segments of the biological sample is compared to a normal control (such as a corresponding normal control ratio X₂ based on genomic segments from non-cancerous tissue). A genomic segment is assigned an altered methylation status if there is an increase in the ratio X₂ compared to the normal control (such as an increase of at least 50% or at least 100%, or at least one standard deviation, or at least two standard deviations compared to the normal control) and a genomic segment is assigned a normal methylation status if there is not an increase in the ratio X₂ compared to the normal control (such as an increase of at least 50% or at least 100%, or at least one standard deviation, or at least two standard deviations compared to the normal control).

In some embodiments, assigning a methylation status to the genomic segments containing chr17:40333009, chr17:46655394, chr6:88876741, chr6:150286508, and chr7:19157193 comprises calculating a ratio X₃ according to: X₃=F₄/(F₃+F₄). F₃ is a frequency of sequence reads in the plurality of sequence reads corresponding to a particular genomic segment where less than 20% (such as less than 10%, less than 5%, or none) of the CpG sites are methylated based on the sequence read. F₄ is a frequency of sequence reads in the plurality corresponding to a particular genomic segment where at least 80% (such as at least 90%, at least 95%, or all) of the CpG sites are methylated based on the sequence read. The ratio X₃ calculated for the sequence reads of genomic segments of the biological sample is compared to a normal control (such as a corresponding normal control ratio X₃ based on genomic segments from non-cancerous tissue). A genomic segment is assigned an altered methylation status if there is an increase in the ratio X₃ compared to the normal control (such as an increase of at least 50% or at least 100%, or at least one standard deviation, or at least two standard deviations compared to the normal control) and a genomic segment is assigned a normal methylation status if there is not an increase in the ratio X₃ compared to the normal control (such as an increase of at least 50% or at least 100%, or at least one standard deviation, or at least two standard deviations compared to the normal control).

In some embodiments, assigning a methylation status to the genomic segments containing chr10:14816201, chr12:129822259, and chr14:89628169 comprises calculating a ratio X₄ according to: X₄=F₃/(F₃+F₄). F₃ is a frequency of sequence reads in the plurality of sequence reads corresponding to a particular genomic segment where less than 20% (such as less than 10%, less than 5%, or none) of the CpG sites are methylated based on the sequence read. F₄ is a frequency of sequence reads in the plurality corresponding to a particular genomic segment where at least 80% (such as at least 90%, at least 95%, or all) of the CpG sites are methylated based on the sequence read. The ratio X₄ calculated for the sequence reads of genomic segments of the biological sample is compared to a normal control (such as a corresponding normal control ratio X₄ based on genomic segments from non-cancerous tissue). A genomic segment is assigned an altered methylation status if there is an increase in the ratio X₄ compared to the normal control (such as an increase of at least 50% or at least 100%, or at least one standard deviation, or at least two standard deviations compared to the normal control) and a genomic segment is assigned a normal methylation status if there is not an increase in the ratio X₄ compared to the normal control (such as an increase of at least 50% or at least 100%, or at least one standard deviation, or at least two standard deviations compared to the normal control).

In some embodiments, assigning a methylation status to the genomic segments containing chr17:40333009, chr17:46655394, chr6:88876741, chr6:150286508, and chr7:19157193 comprises calculating a ratio X₅ according to: X₅=F₆/(F₅+F₆). F₅ is a frequency of sequence reads in the plurality of sequence reads corresponding to a particular genomic segment where none of the CpG sites are methylated based on the sequence read. F₆ is a frequency of sequence reads in the plurality corresponding to a particular genomic segment where all of the CpG sites are methylated based on the sequence read. The ratio X₅ calculated for the sequence reads of genomic segments of the biological sample is compared to a normal control (such as a corresponding normal control ratio X₅ based on genomic segments from non-cancerous tissue). A genomic segment is assigned an altered methylation status if there is an increase in the ratio X₅ compared to the normal control (such as an increase of at least 50% or at least 100%, or at least one standard deviation, or at least two standard deviations compared to the normal control) and a genomic segment is assigned a normal methylation status if there is not an increase in the ratio X₅ compared to the normal control (such as an increase of at least 50% or at least 100%, or at least one standard deviation, or at least two standard deviations compared to the normal control).

In some embodiments, assigning a methylation status to the genomic segments containing chr10:14816201, chr12:129822259, and chr14:89628169 comprises calculating a ratio X₆ according to: X₆=F₅/(F₅+F₆). F₅ is a frequency of sequence reads in the plurality of sequence reads corresponding to a particular genomic segment where none of the CpG sites are methylated based on the sequence read. F₆ is a frequency of sequence reads in the plurality corresponding to a particular genomic segment where all of the CpG sites are methylated based on the sequence read. The ratio X₆ calculated for the sequence reads of genomic segments of the biological sample is compared to a normal control (such as a corresponding normal control ratio X₆ based on genomic segments from non-cancerous tissue). A genomic segment is assigned an altered methylation status if there is an increase in the ratio X₆ compared to the normal control (such as an increase of at least 50% or at least 100%, or at least one standard deviation, or at least two standard deviations compared to the normal control) and a genomic segment is assigned a normal methylation status if there is not an increase in the ratio X₆ compared to the normal control (such as an increase of at least 50% or at least 100%, or at least one standard deviation, or at least two standard deviations compared to the normal control).

In several embodiments, methylation of CpG sites within the eight tumor detection genomic segments is detected using bisulfite-amplicon sequencing (see, e.g., Frommer, et al., Proc Natl Acad Sci USA 89(5): 1827-31, 1992; Feil, et al., Nucleic Acids Res. 22(4): 695-6, 1994). Bisulfite-amplicon sequencing involves treating genomic DNA from a sample with bisulfite to convert unmethylated cytosine to uracil followed by amplification (such as PCR amplification) of a target nucleic acid (such as a target nucleic acid comprising or consisting of any one of the chr6:88876741, chr6:150286508, chr7:19157193; chr10:14816201, chr12:129822259, chr14:89628169, chr17:40333009, or chr17:46655394 genomic segments provided herein) within the treated genomic DNA, and sequencing of the resulting amplicon. Sequencing produces reads that can be aligned to a genomic reference sequence that can be used to quantitate methylation levels of all the CpGs within an amplicon. Cytosines in non-CpG context can be used to track bisulfite conversion efficiency for each individual sample. The procedure is both time and cost-effective, as multiple samples can be sequenced in parallel using a 96 well plate, and generates reproducible measurements of methylation when assayed in independent experiments.

An appropriate primer pair for amplifying the amplicon (such as a target nucleic acid comprising or consisting of any one of the chr6:88876741, chr6:150286508, chr7:19157193; chr10:14816201, chr12:129822259, chr14:89628169, chr17:40333009, or chr17:46655394 genomic segments) is selected. In some embodiments, amplifying the chr6:88876741 genomic segment comprises forward and reverse primers comprising or consisting of SEQ ID NOs: 1 and 2, respectively. In some embodiments, amplifying the chr6:150286508 genomic segment comprises forward and reverse primers comprising or consisting of SEQ ID NOs: 3 and 4, respectively. In some embodiments, amplifying the chr7:19157193 genomic segment comprises forward and reverse primers comprising or consisting of SEQ ID NOs: 5 and 6, respectively. In some embodiments, amplifying the chr10:14816201 genomic segment comprises forward and reverse primers comprising or consisting of SEQ ID NOs: 7 and 8, respectively. In some embodiments, amplifying the chr12:129822259 genomic segment comprises forward and reverse primers comprising or consisting of SEQ ID NOs: 9 and 10, respectively. In some embodiments, amplifying the chr14:89628169 genomic segment comprises forward and reverse primers comprising or consisting of SEQ ID NOs: 11 and 12, respectively. In some embodiments, amplifying the chr17:40333009 genomic segment comprises forward and reverse primers comprising or consisting of SEQ ID NOs: 13 and 14, respectively. In some embodiments, amplifying the chr17:46655394 genomic segment comprises forward and reverse primers comprising or consisting of SEQ ID NOs: 15 and 16, respectively.

In some embodiments, a multiplex amplification assay is performed where multiple primer pairs are used to amplify two or more (such as 2, 3, 4, 5, 6, 7, or all 8) of the genomic segments. In some embodiments, two multiplex amplification reactions are performed to amplify all eight genomic segments, with four genomic segments amplified in each amplification reaction. The primers for use in the amplification reactions can have a maximum length, such as no more than 75 nucleotides in length (for example, no more than 50 nucleotides in length). In several embodiments, the forward and/or reverse primers can be labeled (for example, with adapter sequences or barcode sequences) to facilitate sequencing or purification of the amplicons.

Bisulfite-amplicon sequencing potentially recovers all read patterns present in the sample and allows a more detailed analysis of methylation. Using this approach, altered or normal methylation of the eight genomic segments may be utilized as a pan-cancer biomarker for ctDNA in methods for diagnosing tumors and/or to track effectiveness of chemotherapy from the blood.

Another factor that may help in distinguishing tumors from normals is spiking in internal DNA standards to quantify DNA concentration in blood. That information can be used to quantify the number of methylated reads in unit volume of blood, which serves as a useful additional discriminative tumor signature. Other absolute quantification methods, like ddPCR (digital droplet PCR), may be used as well.

Any suitable amplification methodology can be utilized to selectively or non-selectively amplify one or more of the eight genomic segments from a sample according to the methods provided herein. It will be appreciated that any of the amplification methodologies described herein or generally known in the art can be utilized with target-specific primers to selectively amplify a nucleic acid molecule of interest. Suitable methods for selective amplification include, but are not limited to, the polymerase chain reaction (PCR), strand displacement amplification (SDA), transcription mediated amplification (TMA) and nucleic acid sequence based amplification (NASBA), degenerate oligonucleotide primed polymerase chain reaction (DOP-PCR), primer-extension preamplification polymerase chain reaction (PEP-PCR). The above amplification methods can be employed to selectively amplify one or more nucleic acids of interest. For example, PCR, including multiplex PCR, SDA, TMA, NASBA, DOP-PCR, PEP-PCR, and the like can be utilized to selectively amplify one or more nucleic acids of interest. In such embodiments, primers directed specifically to the nucleic acid of interest are included in the amplification reaction. In some embodiments, selectively amplifying can include one or more non-selective amplification steps. For example, an amplification process using random or degenerate primers can be followed by one or more cycles of amplification using target-specific primers.

In some embodiments presented herein, the methods comprise carrying out one or more sequencing reactions to generate sequence reads of at least a portion of a nucleic acid such as an amplified nucleic acid molecule (e.g. an amplicon or copy of a template nucleic acid). The identity of nucleic acid molecules can be determined based on the sequencing information. Paired-end sequencing allows the determination of two reads of sequence from two places on a single polynucleotide template. One advantage of the paired-end approach is that although a sequencing read may not be long enough to sequence an entire target nucleic acid, significant information can be gained from sequencing two stretches from each end of a single template.

In some embodiments of the methods provided herein, one or more copies of the eight genomic segments from bisulfite treated genomic DNA is sequenced a plurality of times. It can be advantageous to perform repeated sequencing of an amplified nucleic acid molecule in order to ensure a redundancy sufficient to overcome low accuracy base calls. Because sequencing error rates often become higher with longer read lengths, redundancy of sequencing any given nucleotide can enhance sequencing accuracy.

The number of sequencing reads of a nucleotide or nucleic acid is referred to as sequencing depth. In some embodiments, a sequencing read of at least the first region or second region of the amplified exon pair is performed to a depth of at least 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 900, 950 or at least 1000×. In typical embodiments, the accuracy in determining methylation of a genomic DNA sample increases proportionally with the number of reads.

The sequencing reads described herein may be obtained using any suitable sequencing methodology, such as direct sequencing, including sequencing by synthesis (SBS), sequencing by hybridization, and the like. Exemplary SBS procedures, fluidic systems and detection platforms that can be readily adapted for use with amplicons produced by the methods of the present disclosure are described, for example, in Bentley et al., Nature 456:53-59 (2008), WO 04/018497; U.S. Pat. No. 7,057,026; WO 91/06678; WO 07/123,744; U.S. Pat. Nos. 7,329,492; 7,211,414; 7,315,019; 7,405,281, and US 2008/0108082, each of which is incorporated herein by reference. An exemplary sequencing system for use with the disclosed methods is the Illumina MiSeq platform.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568 and 6,274,320, each of which is incorporated herein by reference).

Alternative methods to assay the methylation status of CpG sites can also be used. Numerous DNA methylation detection methods are known in the art, including but not limited to: methylation-specific enzyme digestion (Singer-Sam, et al., Nucleic Acids Res. 18(3): 687, 1990; Taylor, et al., Leukemia 15(4): 583-9, 2001), methylation-specific PCR (MSP or MSPCR) (Herman, et al., Proc Natl Acad Sci USA 93(18): 9821-6, 1996), methylation-sensitive single nucleotide primer extension (MS-SnuPE) (Gonzalgo, et al., Nucleic Acids Res. 25(12): 2529-31, 1997), restriction landmark genomic scanning (RLGS) (Kawai, Mol Cell Biol. 14(11): 7421-7, 1994; Akama, et al., Cancer Res. 57(15): 3294-9, 1997), and differential methylation hybridization (DMH) (Huang, et al., Hum Mol Genet. 8(3): 459-70, 1999). See also the following issued U.S. Pat. Nos. 7,229,759; 7,144,701; b 7,125,857; 7,118,868; 6,960,436; 6,905,669; 6,605,432; 6,265,171; 5,786,146; 6,017,704; and 6,200,756; each of which is incorporated herein by reference.

In some embodiments, the method of detecting cancer comprises providing a biological sample containing cell-free DNA from a human subject, treating the sample with bisulfite, and amplifying the genomic segments containing chr6:88876741, chr6:150286508, chr7:19157193; chr10:14816201, chr12:129822259, chr14:89628169, chr17:40333009, and chr17:46655394 from the bisulfite-treated sample. The methylation of CpG sites in the cell-free DNA is then detected by analyzing the amplified genomic segments using any appropriate procedure, and a methylation status of altered or normal is assigned to the genomic segments. If at least one (such as at least two, or at least three) of the genomic segments is assigned an altered methylation status, then the biological sample is identified as from a subject with cancer. If none of the genomic segments are assigned an altered methylation status, then the biological sample is identified as from a subject without cancer

In several embodiments, the amplification reaction comprises PCR, such as a single multiplex PCR amplification including amplification of each of the genomic segments.

In some such embodiments, amplifying the chr6:88876741 genomic segment comprises forward and reverse primers comprising or consisting of SEQ ID NOs: 1 and 2, respectively. In some embodiments, amplifying the chr6:150286508 genomic segment comprises forward and reverse primers comprising or consisting of SEQ ID NOs: 3 and 4, respectively. In some embodiments, amplifying the chr7:19157193 genomic segment comprises forward and reverse primers comprising or consisting of SEQ ID NOs: 5 and 6, respectively. In some embodiments, amplifying the chr10:14816201 genomic segment comprises forward and reverse primers comprising or consisting of SEQ ID NOs: 7 and 8, respectively. In some embodiments, amplifying the chr12:129822259 genomic segment comprises forward and reverse primers comprising or consisting of SEQ ID NOs: 9 and 10, respectively. In some embodiments, amplifying the chr14:89628169 genomic segment comprises forward and reverse primers comprising or consisting of SEQ ID NOs: 11 and 12, respectively. In some embodiments, amplifying the chr17:40333009 genomic segment comprises forward and reverse primers comprising or consisting of SEQ ID NOs: 13 and 14, respectively. In some embodiments, amplifying the chr17:46655394 genomic segment comprises forward and reverse primers comprising or consisting of SEQ ID NOs: 15 and 16, respectively.

Any appropriate method can be used to detect the methylation of the cell-free DNA corresponding the amplified genomic segments comprises sequencing the amplified genomic segments. In some embodiments, detecting methylation of the cell-free DNA corresponding the amplified genomic segments comprises sequencing the amplicons of the bisulfite-treated cell free DNA. In other embodiments, the amplicons are subjected to a high-resolution PCR melt assay (such as the DREAMing method described in Pisanic et al., “Dreaming a simple and ultrasensitive method for assessing intratumor epigenetic heterogeneity directly from liquid biopsies,” Nucleic Acids Research, 43(22):e154, 2015, which is incorporated by reference herein) to determine methylation status.

Once the methylation of the methylation of the cell-free DNA corresponding the amplified genomic segments is detected, a methylation status of normal or altered is assigned to the genomic segments (for example, as discussed above), and the sample is identified as from a subject with or without cancer.

In another aspect, reagents and kits are provided for bisulfite amplicon sequencing of the eight genomic segments as provided herein. The kits include forward and reverse primers to amplify the genomic segments. In some embodiments, the kit can include one or more containers containing forward and/or reverse primers for amplifying one or more target nucleic acid molecule comprising or consisting of one or more of the genomic segments. The target nucleic acid molecule can have a maximum length, for example no more than 1000 (such as no more than 750, no more than 500, no more than 400, or no more than 350) nucleotides in length. In some embodiments, also included are sodium bisulfite reagents as well as reagents used for amplicon sequencing. The kit may also include adapter sequences for the amplicon.

In some embodiments, the kit includes one or more (such as 1, 2, 3, 4, 5, 6, 7, or all 8) primers comprising the amino acid sequence of any of SEQ ID NOs: 1-16, wherein the primers are up to 75 (such as up to 50) nucleotides in length. In some embodiments, the kit includes a primer for each of the amino acid sequence of any of SEQ ID NOs: 1-16, wherein the primers are up to 75 (such as up to 50) nucleotides in length. In some embodiments, the primers in the kit consist of the amino acid sequences set forth as SEQ ID NOs: 1-16. The primers can be labelled with a detectable marker as needed for the intended purpose of the kit, such as dyes and fluorescent markers for detection in a PCT assay.

Following detection of cancer in a subject, any appropriate treatment can be administered to the subject to inhibit or reduce the cancer, such as surgical removal of the cancer and/or administration of a therapeutically effective amount of one or more anti-cancer agents and/or a radiotherapy and/or a chemotherapy to the subject to treat the cancer in the subject. In some embodiments, the subject identified as with cancer as described above is treated by performing frequent monitoring for the cancer, for example by ultrasound imaging, CT imaging, MRI imaging, PET scan or digital rectal exam. In some embodiments, the subject has a prior history of the cancer, and identifying the subject as having cancer as described herein identifies a relapse or a high risk of relapse of the cancer and the subject is treated with is treated by performing frequent monitoring for the cancer, for example by ultrasound imaging, CT imaging, MRI imaging, PET scan or digital rectal exam.

### IV. Classifying Cancer

The present disclosure relates to classification of cancer type in a subject using DNA methylation of specific segments of genomic DNA from the subject as a biomarker. Having identified the specified segments as a highly sensitive and specific cancer type markers, methods of classifying cancer in a subject and/or a biological sample from the subject are provided.

As disclosed herein, the methylation state of cytosines within a panel of 39 genomic segments can be used as a biomarker for classification of the cancer type in a subject, and to identify a biological sample from a subject with particular cancer type. The genomic segments in the panel contain the following genomic positions: chr8:102451058, chr19:16189360, chr2:114035619, chr10:5566908, chr16:678127, chr6:106958645, chr4:142054417, chr10:116064472, chr11:60619955, chr16:51184392, chr2:8724060, chr13:113424938, chr2:240270793, chr2:219256101, chr11:8284312, chr19:1827498, chr19:18335182, chr9:140683797, chr10:21788638, chr8:1895558, chr7:27196759, chr7:4801993, chr10:114591733, chr4:156588387, chr10:1120831, chr12:54427173, chr2:25600752, chr10:8097689, chr6:133562470, chr10:8097331, chr10:103603810, chr17:46655394, chr5:140306231, chr2:66665428, chr2:176994448, chr2:61372138, chr2:176994764, chr8:97506675, and chr17:46711341 according to a GRCh37/hg19 reference human genome.

The cancer type classified by the method can be any type of cancer, including but not limited to colon cancer, rectal cancer, stomach cancer, pancreatic cancer, bladder cancer, head-neck cancer, lung cancer, breast cancer, kidney cancer, cervical cancer, liver cancer, uterine cancer, ovarian cancer, and prostate cancer.

Classifying a cancer type in a subject based on the 39 classification probes can include obtaining a biological sample from the subject. The sample can be any sample that includes genomic DNA. Such samples include, but are not limited to, tissue from biopsies (including formalin-fixed paraffin-embedded tissue), autopsies, and pathology specimens; sections of tissues (such as frozen sections or paraffin-embedded sections taken for histological purposes); body fluids, such as blood, sputum, serum, ejaculate, or urine, or fractions of any of these; and so forth. In one particular example, the sample from the subject is a tissue biopsy sample. In another specific example, the sample from the subject is urine. In some embodiments the biological sample is a plasma or serum sample comprising cell-free DNA. In several embodiments, the biological sample is from a subject suspected of having a cancer, such as colon cancer, rectal cancer, stomach cancer, pancreatic cancer, bladder cancer, head-neck cancer, lung cancer, breast cancer, kidney cancer, cervical cancer, liver cancer, uterine cancer, ovarian cancer, and prostate cancer. In some embodiments, the biological sample is a tumor sample or a suspected tumor sample. For example, the sample can be a biopsy sample from at or near or just beyond the perceived leading edge of a tumor in a subject. Testing of the sample using the methods provided herein can be used to confirm the location of the leading edge of the tumor in the subject. This information can be used, for example, to determine if further surgical removal of tumor tissue is appropriate.

In some embodiments, an amplicon generated from cell-free DNA derived from blood (or a portion thereof, such as plasma or serum) can be used to detect the methylation of circulating tumor DNA (ctDNA). There are many studies detecting and assessing the fraction of ctDNA based on mutations. However, mutation-based detection is only specific to the tumors harboring those mutations and without a detailed understanding of normal samples it is not always clear what levels of ctDNA should be considered abnormal and warrant intervention. Conversely, the methylation state of cytosines within the disclosed genomic segments may be similar throughout different tumor types and may complement or supersede mutation markers for better diagnosis.

In some embodiments, a plurality of sequence reads of a methylation sequencing assay are obtained. The sequence reads cover the panel of 39 genomic segments as provided herein for classifying the type of cancer in a subject, and to identify a biological sample from a subject a particular cancer type. Thus, the sequence reads cover a panel of 39 genomic segments containing the following 39 genomic positions: chr8:102451058, chr19:16189360, chr2:114035619, chr10:5566908, chr16:678127, chr6:106958645, chr4:142054417, chr10:116064472, chr11:60619955, chr16:51184392, chr2:8724060, chr13:113424938, chr2:240270793, chr2:219256101, chr11:8284312, chr19:1827498, chr19:18335182, chr9:140683797, chr10:21788638, chr8:1895558, chr7:27196759, chr7:4801993, chr10:114591733, chr4:156588387, chr10:1120831, chr12:54427173, chr2:25600752, chr10:8097689, chr6:133562470, chr10:8097331, chr10:103603810, chr17:46655394, chr5:140306231, chr2:66665428, chr2:176994448, chr2:61372138, chr2:176994764, chr8:97506675, and chr17:46711341 according to a GRCh37/hg19 reference human genome. A methylation status of altered or normal is assigned to each of the genomic segments by comparing methylation of CpG sites of the sequence reads covering the respective genomic segments to a normal control. The cancer type is classified based on the pattern of methylation status of the genomic segments.

Each genomic segment contains an appropriate amount of contiguous DNA containing the chr8:102451058, chr19:16189360, chr2:114035619, chr10:5566908, chr16:678127, chr6:106958645, chr4:142054417, chr10:116064472, chr11:60619955, chr16:51184392, chr2:8724060, chr13:113424938, chr2:240270793, chr2:219256101, chr11:8284312, chr19:1827498, chr19:18335182, chr9:140683797, chr10:21788638, chr8:1895558, chr7:27196759, chr7:4801993, chr10:114591733, chr4:156588387, chr10:1120831, chr12:54427173, chr2:25600752, chr10:8097689, chr6:133562470, chr10:8097331, chr10:103603810, chr17:46655394, chr5:140306231, chr2:66665428, chr2:176994448, chr2:61372138, chr2:176994764, chr8:97506675, and chr17:46711341 genomic position to capture a sufficient number of the CpG sites surrounding these positions to determine whether or not the genomic segment has an altered or normal methylation status. In some embodiments, each genomic segment independently contains plus or minus up to 300 bases (for example, up to 200 bases, up to 100 bases, or up to 50 bases) of the genomic positions, such as plus or minus 50 to 300 bases of the genomic positions.

Any appropriate method can be used to assign a methylation status of altered or normal to the 39 genomic segments. For example, in some embodiments, a genomic segment is assigned an altered methylation status if the CpG sites of the segment are not methylated or have a low frequency of methylation (such as less than 20%) in non-cancerous (normal) tissue and the CpG sites of the genomic segment from the biological sample are identified as hypermethylated (such as more than 80% of the CpG sites in the genomic segment are methylated). In some embodiments, a genomic segment is assigned an altered methylation status if the CpG sites of the genomic segment are all methylated or have a high frequency of methylation (such as more than 80%) in non-cancerous (normal) tissue and the CpG sites of the genomic segment from the biological sample are identified as hypomethylated (such as less than 20% of the CpG sites in the genomic segment are methylated).

In some embodiments, assigning a methylation status to the genomic segments containing chr10:8097331, chr10:8097689, chr10:103603810, chr10:116064472, chr11:8284312, chr12:54427173, chr16:51184392, chr17:46655394, chr17:46711341, chr19:16189360, chr19:18335182, chr2:8724060, chr2:61372138, chr2:66665428, chr2:114035619, chr2:176994448, chr2:176994764, chr4:142054417, chr4:156588387, chr5:140306231, chr6:106958645, chr6:133562470, chr7:27196759, and chr8:97506675 comprises calculating a ratio Y₁ according to: Y₁=F₂/(F₁+F₂). F₁ is a frequency of sequence reads in the plurality of sequence reads corresponding to a particular genomic segment where less than 40% (such as less than 30%, less than 25%, less than 20%, less than 10%, or none) of the CpG sites are methylated based on the sequence read. F₂ is a frequency of sequence reads in the plurality corresponding to a particular genomic segment where at least 60% (such as at least 70%, at least 80%, at least 90% or all) of the CpG sites are methylated based on the sequence read. The ratio Y₁ calculated for the sequence reads of genomic segments of the biological sample is compared to a normal control (such as a corresponding normal control ratio Y₁ based on genomic segments from non-cancerous tissue). A genomic segment is assigned an altered methylation status if there is an increase in the ratio Y₁ compared to the normal control (such as an increase of at least 50% or at least 100%, or at least one standard deviation, or at least two standard deviations compared to the normal control) and a genomic segment is assigned a normal methylation status if there is not an increase in the ratio Y₁ compared to the normal control (such as an increase of at least 50% or at least 100%, or at least one standard deviation, or at least two standard deviations compared to the normal control).

In some embodiments, assigning a methylation status to the genomic segments containing chr10:1120831, chr10:5566908, chr10:21788638, chr10:114591733, chr11:60619955, chr13:113424938, chr16:678127, chr19:1827498, chr2:25600752, chr2:219256101, chr2:240270793, chr7:4801993, chr8:1895558, chr8:102451058, and chr9:140683797 comprises calculating a ratio Y₂ according to: Y₂=F₁/(F₁+F₂). F₁ is a frequency of sequence reads in the plurality of sequence reads corresponding to a particular genomic segment where less than 40% (such as less than 30%, less than 20%, less than 10%, or none) of the CpG sites are methylated based on the sequence read. F₂ is a frequency of sequence reads in the plurality corresponding to a particular genomic segment where at least 60% (such as at least 70%, at least 80% at least 90%, or all) of the CpG sites are methylated based on the sequence read. The ratio Y₂ calculated for the sequence reads of genomic segments of the biological sample is compared to a normal control (such as a corresponding normal control ratio Y₂ based on genomic segments from non-cancerous tissue). A genomic segment is assigned an altered methylation status if there is an increase in the ratio Y₂ compared to the normal control (such as an increase of at least 50% or at least 100%, or at least one standard deviation, or at least two standard deviations compared to the normal control) and a genomic segment is assigned a normal methylation status if there is not an increase in the ratio Y₂ compared to the normal control (such as an increase of at least 50% or at least 100%, or at least one standard deviation, or at least two standard deviations compared to the normal control).

In some embodiments, assigning a methylation status to the genomic segments containing chr10:8097331, chr10:8097689, chr10:103603810, chr10:116064472, chr11:8284312, chr12:54427173, chr16:51184392, chr17:46655394, chr17:46711341, chr19:16189360, chr19:18335182, chr2:8724060, chr2:61372138, chr2:66665428, chr2:114035619, chr2:176994448, chr2:176994764, chr4:142054417, chr4:156588387, chr5:140306231, chr6:106958645, chr6:133562470, chr7:27196759, and chr8:97506675 comprises calculating a ratio Y₃ according to: Y₃=F₄/(F₃+F₄). F₃ is a frequency of sequence reads in the plurality of sequence reads corresponding to a particular genomic segment where less than 20% (such as less than 10%, less than 5%, or none) of the CpG sites are methylated based on the sequence read. F₄ is a frequency of sequence reads in the plurality corresponding to a particular genomic segment where at least 80% (such as at least 90%, at least 95%, or all) of the CpG sites are methylated based on the sequence read. The ratio Y₃ calculated for the sequence reads of genomic segments of the biological sample is compared to a normal control (such as a corresponding normal control ratio Y₃ based on genomic segments from non-cancerous tissue). A genomic segment is assigned an altered methylation status if there is an increase in the ratio Y₃ compared to the normal control (such as an increase of at least 50% or at least 100%, or at least one standard deviation, or at least two standard deviations compared to the normal control) and a genomic segment is assigned a normal methylation status if there is not an increase in the ratio Y₃ compared to the normal control (such as an increase of at least 50% or at least 100%, or at least one standard deviation, or at least two standard deviations compared to the normal control).

In some embodiments, assigning a methylation status to the genomic segments containing chr10:1120831, chr10:5566908, chr10:21788638, chr10:114591733, chr11:60619955, chr13:113424938, chr16:678127, chr19:1827498, chr2:25600752, chr2:219256101, chr2:240270793, chr7:4801993, chr8:1895558, chr8:102451058, and chr9:140683797 comprises calculating a ratio Y₄ according to: Y₄=F₃/(F₃+F₄). F₃ is a frequency of sequence reads in the plurality of sequence reads corresponding to a particular genomic segment where less than 20% (such as less than 10%, less than 5%, or none) of the CpG sites are methylated based on the sequence read. F₄ is a frequency of sequence reads in the plurality corresponding to a particular genomic segment where at least 80% (such as at least 90%, at least 95%, or all) of the CpG sites are methylated based on the sequence read. The ratio Y₄ calculated for the sequence reads of genomic segments of the biological sample is compared to a normal control (such as a corresponding normal control ratio Y₄ based on genomic segments from non-cancerous tissue). A genomic segment is assigned an altered methylation status if there is an increase in the ratio Y₄ compared to the normal control (such as an increase of at least 50% or at least 100%, or at least one standard deviation, or at least two standard deviations compared to the normal control) and a genomic segment is assigned a normal methylation status if there is not an increase in the ratio Y₄ compared to the normal control (such as an increase of at least 50% or at least 100%, or at least one standard deviation, or at least two standard deviations compared to the normal control).

In some embodiments, assigning a methylation status to the genomic segments containing chr10:8097331, chr10:8097689, chr10:103603810, chr10:116064472, chr11:8284312, chr12:54427173, chr16:51184392, chr17:46655394, chr17:46711341, chr19:16189360, chr19:18335182, chr2:8724060, chr2:61372138, chr2:66665428, chr2:114035619, chr2:176994448, chr2:176994764, chr4:142054417, chr4:156588387, chr5:140306231, chr6:106958645, chr6:133562470, chr7:27196759, and chr8:97506675 comprises calculating a ratio Y₅ according to: Y₅=F₆/(F₅+F₆). F₅ is a frequency of sequence reads in the plurality of sequence reads corresponding to a particular genomic segment where none of the CpG sites are methylated based on the sequence read. F₆ is a frequency of sequence reads in the plurality corresponding to a particular genomic segment where all of the CpG sites are methylated based on the sequence read. The ratio Y₅ calculated for the sequence reads of genomic segments of the biological sample is compared to a normal control (such as a corresponding normal control ratio Y₅ based on genomic segments from non-cancerous tissue). A genomic segment is assigned an altered methylation status if there is an increase in the ratio Y₅ compared to the normal control (such as an increase of at least 50% or at least 100%, or at least one standard deviation, or at least two standard deviations compared to the normal control) and a genomic segment is assigned a normal methylation status if there is not an increase in the ratio Y₅ compared to the normal control (such as an increase of at least 50% or at least 100%, or at least one standard deviation, or at least two standard deviations compared to the normal control).

In some embodiments, assigning a methylation status to the genomic segments containing chr10:1120831, chr10:5566908, chr10:21788638, chr10:114591733, chr11:60619955, chr13:113424938, chr16:678127, chr19:1827498, chr2:25600752, chr2:219256101, chr2:240270793, chr7:4801993, chr8:1895558, chr8:102451058, and chr9:140683797 comprises calculating a ratio Y₆ according to: Y₆=F₆/(F₅+F₆). F₃ is a frequency of sequence reads in the plurality of sequence reads corresponding to a particular genomic segment where none of the CpG sites are methylated based on the sequence read. F₆ is a frequency of sequence reads in the plurality corresponding to a particular genomic segment where all of the CpG sites are methylated based on the sequence read. The ratio Y₆ calculated for the sequence reads of genomic segments of the biological sample is compared to a normal control (such as a corresponding normal control ratio Y₆ based on genomic segments from non-cancerous tissue). A genomic segment is assigned an altered methylation status if there is an increase in the ratio Y₆ compared to the normal control (such as an increase of at least 50% or at least 100%, or at least one standard deviation, or at least two standard deviations compared to the normal control) and a genomic segment is assigned a normal methylation status if there is not an increase in the ratio Y₆ compared to the normal control (such as an increase of at least 50% or at least 100%, or at least one standard deviation, or at least two standard deviations compared to the normal control).

Classifying the type of cancer in the subject into one of the plurality of different cancer types comprises comparing the methylation status of the genomic segments of the biological sample to the cancer type control. In several embodiments, a distance calculation (such as mean Euclidean distance, quantile normalization, or naïve Bayes) is used to compare the methylation status of the genomic segments of the biological sample to the cancer type control.

In some embodiments, a biological sample is classified as from a subject with colon and/or rectum cancer if the genomic segments containing chr2:114035619, chr4:142054417, chr11:60619955, chr16:51184392, chr2:240270793, chr11:8284312, chr9:140683797, chr4:156588387, chr2:66665428, chr2:61372138, and chr8:97506675 have an altered methylation status and the remaining tumor classification genomic segments have a normal methylation status, and/or the methylation status of altered or normal assigned to the 39 tumor classification genomic segments has pattern with minimal distance to chr2:114035619, chr4:142054417, chr11:60619955, chr16:51184392, chr2:240270793, chr11:8284312, chr9:140683797, chr4:156588387, chr2:66665428, chr2:61372138, and chr8:97506675 having an altered methylation status and the remaining genomic segments have a normal methylation status.

In some embodiments, a biological sample is classified as from a subject with stomach cancer the genomic segments containing chr2:114035619, chr4:142054417, chr11:60619955, chr16:51184392, chr11:8284312, chr9:140683797, chr7:27196759, chr4:156588387, chr2:66665428, chr2:176994448, chr2:61372138, chr2:176994764, chr8:97506675, and chr17:46711341 have an altered methylation status and the remaining tumor classification genomic segments have a normal methylation status, and/or the methylation status of altered or normal assigned to the 39 tumor classification genomic segments has pattern with minimal distance to chr2:114035619, chr4:142054417, chr11:60619955, chr16:51184392, chr11:8284312, chr9:140683797, chr7:27196759, chr4:156588387, chr2:66665428, chr2:176994448, chr2:61372138, chr2:176994764, chr8:97506675, and chr17:46711341 having an altered methylation status and the remaining genomic segments have a normal methylation status.

In some embodiments, a biological sample is classified as from a subject with pancreatic cancer the genomic segments containing chr2:114035619, chr11:60619955, chr16:51184392, chr9:140683797, chr7:27196759, chr2:176994448, chr2:176994764, and chr17:46711341 have an altered methylation status and the remaining tumor classification genomic segments have a normal methylation status, and/or the methylation status of altered or normal assigned to the 39 tumor classification genomic segments has pattern with minimal distance to chr2:114035619, chr11:60619955, chr16:51184392, chr9:140683797, chr7:27196759, chr2:176994448, chr2:176994764, and chr17:46711341 having an altered methylation status and the remaining genomic segments have a normal methylation status.

In some embodiments, a biological sample is classified as from a subject with bladder cancer the genomic segments containing chr8:102451058, chr2:114035619, chr10:5566908, chr6:106958645, chr16:51184392, chr2:219256101, chr7:27196759, chr6:133562470, chr10:103603810, and chr5:140306231 have an altered methylation status and the remaining tumor classification genomic segments have a normal methylation status, and/or the methylation status of altered or normal assigned to the 39 tumor classification genomic segments has pattern with minimal distance to chr8:102451058, chr2:114035619, chr10:5566908, chr6:106958645, chr16:51184392, chr2:219256101, chr7:27196759, chr6:133562470, chr10:103603810, and chr5:140306231 having an altered methylation status and the remaining genomic segments have a normal methylation status.

In some embodiments, a biological sample is classified as from a subject with head-neck cancer if the genomic segments containing chr8:102451058, chr2:114035619, chr10:5566908, chr6:106958645, chr16:51184392, chr19:18335182, chr7:27196759, chr7:4801993, chr2:25600752, chr10:8097689, chr6:133562470, chr10:8097331, chr10:103603810, chr17:46655394, and chr5:140306231 have an altered methylation status and the remaining tumor classification genomic segments have a normal methylation status, and/or the methylation status of altered or normal assigned to the 39 tumor classification genomic segments has pattern with minimal distance to chr8:102451058, chr2:114035619, chr10:5566908, chr6:106958645, chr16:51184392, chr19:18335182, chr7:27196759, chr7:4801993, chr2:25600752, chr10:8097689, chr6:133562470, chr10:8097331, chr10:103603810, chr17:46655394, and chr5:140306231 having an altered methylation status and the remaining genomic segments have a normal methylation status.

In some embodiments, a biological sample is classified as from a subject with lung squamous cell carcinoma the genomic segments containing chr8:102451058, chr2:114035619, chr10:5566908, chr16:51184392, chr7:27196759, chr7:4801993, chr10:8097689, chr10:8097331, and chr17:46655394 have an altered methylation status and the remaining tumor classification genomic segments have a normal methylation status, and/or the methylation status of altered or normal assigned to the 39 tumor classification genomic segments has pattern with minimal distance to chr8:102451058, chr2:114035619, chr10:5566908, chr16:51184392, chr7:27196759, chr7:4801993, chr10:8097689, chr10:8097331, and chr17:46655394 having an altered methylation status and the remaining genomic segments have a normal methylation status.

In some embodiments, a biological sample is classified as from a subject with lung adenocarcinoma the genomic segments containing chr8:102451058, chr2:114035619, chr16:678127, chr16:51184392, chr13:113424938, chr7:27196759, and chr10:1120831 have an altered methylation status and the remaining tumor classification genomic segments have a normal methylation status, and/or the methylation status of altered or normal assigned to the 39 tumor classification genomic segments has pattern with minimal distance to chr8:102451058, chr2:114035619, chr16:678127, chr16:51184392, chr13:113424938, chr7:27196759, and chr10:1120831 having an altered methylation status and the remaining genomic segments have a normal methylation status.

In some embodiments, a biological sample is classified as from a subject with breast cancer the genomic segments containing chr8:102451058, chr2:114035619, chr16:51184392, chr13:113424938, chr8:1895558, and chr7:27196759 have an altered methylation status and the remaining tumor classification genomic segments have a normal methylation status, and/or the methylation status of altered or normal assigned to the 39 tumor classification genomic segments has pattern with minimal distance to chr8:102451058, chr2:114035619, chr16:51184392, chr13:113424938, chr8:1895558, and chr7:27196759 having an altered methylation status and the remaining genomic segments have a normal methylation status.

In some embodiments, a biological sample is classified as from a subject with kidney cancer the genomic segments containing chr19:16189360, chr16:678127, chr11:60619955, chr19:1827498, chr9:140683797, chr10:21788638, and chr7:27196759 have an altered methylation status and the remaining tumor classification genomic segments have a normal methylation status, and/or the methylation status of altered or normal assigned to the 39 tumor classification genomic segments has pattern with minimal distance to chr19:16189360, chr16:678127, chr11:60619955, chr19:1827498, chr9:140683797, chr10:21788638, and chr7:27196759 having an altered methylation status and the remaining genomic segments have a normal methylation status.

In some embodiments, a biological sample is classified as from a subject with cervical kidney renal papillary cell carcinoma the genomic segments containing chr19:16189360, chr11:60619955, chr9:140683797, chr10:21788638, chr7:27196759, and chr12:54427173 have an altered methylation status and the remaining tumor classification genomic segments have a normal methylation status, and/or the methylation status of altered or normal assigned to the 39 tumor classification genomic segments has pattern with minimal distance to chr19:16189360, chr11:60619955, chr9:140683797, chr10:21788638, chr7:27196759, and chr12:54427173 having an altered methylation status and the remaining genomic segments have a normal methylation status.

In some embodiments, a biological sample is classified as from a subject with liver cancer the genomic segments containing chr19:16189360, chr2:114035619, chr11:60619955, chr2:8724060, chr10:21788638, chr8:1895558, and chr7:27196759 have an altered methylation status and the remaining tumor classification genomic segments have a normal methylation status, and/or the methylation status of altered or normal assigned to the 39 tumor classification genomic segments has pattern with minimal distance to chr19:16189360, chr2:114035619, chr11:60619955, chr2:8724060, chr10:21788638, chr8:1895558, and chr7:27196759 having an altered methylation status and the remaining genomic segments have a normal methylation status.

In some embodiments, a biological sample is classified as from a subject with prostate cancer the genomic segments containing chr8:102451058, chr19:16189360, chr2:114035619, chr10:5566908, chr16:51184392, chr2:8724060, chr2:240270793, chr8:1895558, chr7:27196759, and chr10:114591733 have an altered methylation status and the remaining tumor classification genomic segments have a normal methylation status, and/or the methylation status of altered or normal assigned to the 39 tumor classification genomic segments has pattern with minimal distance to chr8:102451058, chr19:16189360, chr2:114035619, chr10:5566908, chr16:51184392, chr2:8724060, chr2:240270793, chr8:1895558, chr7:27196759, and chr10:114591733 having an altered methylation status and the remaining genomic segments have a normal methylation status.

In some embodiments, a biological sample is classified as from a subject with uterine cancer the genomic segments containing chr8:102451058, chr13:113424938, chr10:21788638, and chr7:27196759 have an altered methylation status and the remaining tumor classification genomic segments have a normal methylation status, and/or the methylation status of altered or normal assigned to the 39 tumor classification genomic segments has pattern with minimal distance to chr8:102451058, chr13:113424938, chr10:21788638, and chr7:27196759 having an altered methylation status and the remaining genomic segments have a normal methylation status.

In several embodiments, methylation of CpG sites within the 39 tumor classification genomic segments is detected using bisulfite-amplicon sequencing (see, e.g., Frommer, et al., Proc Natl Acad Sci USA 89(5): 1827-31, 1992; Feil, et al., Nucleic Acids Res. 22(4): 695-6, 1994). Bisulfite-amplicon sequencing involves treating genomic DNA from a sample with bisulfite to convert unmethylated cytosine to uracil followed by amplification (such as PCR amplification) of a target nucleic acid (such as a target nucleic acid comprising or consisting of any one of the chr8:102451058, chr19:16189360, chr2:114035619, chr10:5566908, chr16:678127, chr6:106958645, chr4:142054417, chr10:116064472, chr11:60619955, chr16:51184392, chr2:8724060, chr13:113424938, chr2:240270793, chr2:219256101, chr11:8284312, chr19:1827498, chr19:18335182, chr9:140683797, chr10:21788638, chr8:1895558, chr7:27196759, chr7:4801993, chr10:114591733, chr4:156588387, chr10:1120831, chr12:54427173, chr2:25600752, chr10:8097689, chr6:133562470, chr10:8097331, chr10:103603810, chr17:46655394, chr5:140306231, chr2:66665428, chr2:176994448, chr2:61372138, chr2:176994764, chr8:97506675, and chr17:46711341 genomic segments provided herein) within the treated genomic DNA, and sequencing of the resulting amplicon. Sequencing produces reads that can be aligned to a genomic reference sequence that can be used to quantitate methylation levels of all the CpGs within an amplicon. Cytosines in non-CpG context can be used to track bisulfite conversion efficiency for each individual sample. The procedure is both time and cost-effective, as multiple samples can be sequenced in parallel using a 96 well plate, and generates reproducible measurements of methylation when assayed in independent experiments.

An appropriate primer pair for amplifying the amplicon (such as a target nucleic acid comprising or consisting of any one of the chr8:102451058, chr19:16189360, chr2:114035619, chr10:5566908, chr16:678127, chr6:106958645, chr4:142054417, chr10:116064472, chr11:60619955, chr16:51184392, chr2:8724060, chr13:113424938, chr2:240270793, chr2:219256101, chr11:8284312, chr19:1827498, chr19:18335182, chr9:140683797, chr10:21788638, chr8:1895558, chr7:27196759, chr7:4801993, chr10:114591733, chr4:156588387, chr10:1120831, chr12:54427173, chr2:25600752, chr10:8097689, chr6:133562470, chr10:8097331, chr10:103603810, chr17:46655394, chr5:140306231, chr2:66665428, chr2:176994448, chr2:61372138, chr2:176994764, chr8:97506675, and chr17:46711341 genomic segments) is selected. In some embodiments, a multiplex amplification assay is performed where multiple primer pairs are used to amplify two or more (such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or all 39) of the genomic segments. In some embodiments, two or more multiplex amplification reactions are performed to amplify all 39 genomic segments, with a portion (such as four or five) of the genomic segments amplified in each amplification reaction. The primers for use in the amplification reactions can have a maximum length, such as no more than 75 nucleotides in length (for example, no more than 50 nucleotides in length). In several embodiments, the forward and/or reverse primers can be labeled (for example, with adapter sequences or barcode sequences) to facilitate sequencing or purification of the amplicons.

Bisulfite-amplicon sequencing potentially recovers all read patterns present in the sample and allows a more detailed analysis of methylation. Using this approach, altered or normal methylation of the 39 tumor classification genomic segments may be utilized to assess cancer type across a wide variety of different cancers for diagnosing cancer type from the blood. Another factor that may help in classifying tumor type is spiking in internal DNA standards to quantify DNA concentration in blood. That information can be used to quantify the number of methylated reads in unit volume of blood, which serves as a useful additional discriminative tumor signature. Other absolute quantification methods, like ddPCR (digital droplet PCR), may be used as well.

Any suitable amplification methodology can be utilized to selectively or non-selectively amplify one or more of the 39 tumor classification genomic segments from a sample according to the methods provided herein. It will be appreciated that any of the amplification methodologies described herein or generally known in the art can be utilized with target-specific primers to selectively amplify a nucleic acid molecule of interest. Suitable methods for selective amplification include, but are not limited to, the polymerase chain reaction (PCR), strand displacement amplification (SDA), transcription mediated amplification (TMA) and nucleic acid sequence based amplification (NASBA), degenerate oligonucleotide primed polymerase chain reaction (DOP-PCR), primer-extension preamplification polymerase chain reaction (PEP-PCR). The above amplification methods can be employed to selectively amplify one or more nucleic acids of interest. For example, PCR, including multiplex PCR, SDA, TMA, NASBA, DOP-PCR, PEP-PCR, and the like can be utilized to selectively amplify one or more nucleic acids of interest. In such embodiments, primers directed specifically to the nucleic acid of interest are included in the amplification reaction. In some embodiments, selectively amplifying can include one or more non-selective amplification steps. For example, an amplification process using random or degenerate primers can be followed by one or more cycles of amplification using target-specific primers.

In some embodiments presented herein, the methods comprise carrying out one or more sequencing reactions to generate sequence reads of at least a portion of a nucleic acid such as an amplified nucleic acid molecule (e.g., an amplicon or copy of a template nucleic acid). The identity of nucleic acid molecules can be determined based on the sequencing information. Paired-end sequencing allows the determination of two reads of sequence from two places on a single polynucleotide template. One advantage of the paired-end approach is that although a sequencing read may not be long enough to sequence an entire target nucleic acid, significant information can be gained from sequencing two stretches from each end of a single template.

In some embodiments of the methods provided herein, one or more copies of the 39 tumor classification genomic segments from bisulfite treated genomic DNA is sequenced a plurality of times. It can be advantageous to perform repeated sequencing of an amplified nucleic acid molecule in order to ensure a redundancy sufficient to overcome low accuracy base calls. Because sequencing error rates often become higher with longer read lengths, redundancy of sequencing any given nucleotide can enhance sequencing accuracy.

The number of sequencing reads of a nucleic acid is referred to as sequencing depth. In some embodiments, a sequencing read of the 39 tumor classification genomic segments is performed to a depth of at least 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 900, 950 or at least 1000×. In typical embodiments, the accuracy in determining methylation of a genomic DNA sample increases proportionally with the number of reads.

The sequencing reads of the 39 tumor classification genomic segments described herein may be obtained using any suitable sequencing methodology, such as direct sequencing, including sequencing by synthesis (SBS), sequencing by hybridization, and the like. Exemplary SBS procedures, fluidic systems and detection platforms that can be readily adapted for use with amplicons produced by the methods of the present disclosure are described, for example, in Bentley et al., Nature 456:53-59 (2008), WO 04/018497; U.S. Pat. No. 7,057,026; WO 91/06678; WO 07/123,744; U.S. Pat. Nos. 7,329,492; 7,211,414; 7,315,019; 7,405,281, and US 2008/0108082, each of which is incorporated herein by reference. An exemplary sequencing system for use with the disclosed methods is the Illumina MiSeq platform.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568 and 6,274,320, each of which is incorporated herein by reference).

Alternative methods to assay the methylation status of CpG sites within the 39 tumor classification genomic segments can also be used. Numerous DNA methylation detection methods are known in the art, including but not limited to: methylation-specific enzyme digestion (Singer-Sam, et al., Nucleic Acids Res. 18(3): 687, 1990; Taylor, et al., Leukemia 15(4): 583-9, 2001), methylation-specific PCR (MSP or MSPCR) (Herman, et al., Proc Natl Acad Sci USA 93(18): 9821-6, 1996), methylation-sensitive single nucleotide primer extension (MS-SnuPE) (Gonzalgo, et al., Nucleic Acids Res. 25(12): 2529-31, 1997), restriction landmark genomic scanning (RLGS) (Kawai, Mol Cell Biol. 14(11): 7421-7, 1994; Akama, et al., Cancer Res. 57(15): 3294-9, 1997), and differential methylation hybridization (DMH) (Huang, et al., Hum Mol Genet. 8(3): 459-70, 1999). See also the following issued U.S. Pat. Nos. 7,229,759; 7,144,701; b 7,125,857; 7,118,868; 6,960,436; 6,905,669; 6,605,432; 6,265,171; 5,786,146; 6,017,704; and 6,200,756; each of which is incorporated herein by reference.

In another aspect, reagents and kits are provided for bisulfite amplicon sequencing of the 39 tumor classification genomic segments as provided herein. The kits include forward and reverse primers to amplify the genomic segments. In some embodiments, the kit can include one or more containers containing forward and/or reverse primers for amplifying one or more target nucleic acid molecule comprising or consisting of one or more of the genomic segments. The target nucleic acid molecule can have a maximum length, for example no more than 1000 (such as no more than 750, no more than 500, no more than 400, or no more than 350) nucleotides in length. In some embodiments, also included are sodium bisulfite reagents as well as reagents used for amplicon sequencing. The kit may also include adapter sequences for the amplicon.

Following classification of the cancer in a subject, any appropriate treatment can be administered to the subject to inhibit or reduce the classified cancer, such as surgical removal of the cancer and/or administration of a therapeutically effective amount of one or more anti-cancer agents to the subject to treat the cancer in the subject.

### III. Computer Implemented Embodiments

The analytic methods described herein can be implemented by use of computer systems. For example, any of the steps described above for evaluating sequence reads to determine methylation status of a CpG site may be performed by means of software components loaded into a computer or other information appliance or digital device. When so enabled, the computer, appliance or device may then perform all or some of the above-described steps to assist the analysis of values associated with the methylation of a one or more CpG sites, or for comparing such associated values. The above features embodied in one or more computer programs may be performed by one or more computers running such programs.

Aspects of the disclosed methods for identifying a biological sample from a subject with cancer or classifying the type of cancer can be implemented using computer-based calculations and tools. For example, a methylation status for a CpG site can be assigned by a computer based on an underlying sequence read of an amplicon from a bisulfite amplicon sequencing assay. In another example, a methylation status for a genomic segment as provided herein can be compared by a computer to a threshold value, as described herein. The tools are advantageously provided in the form of computer programs that are executable by a general purpose computer system (for example, as described in the following section) of conventional design.

Computer code for implementing aspects of the present invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages. The host computer system advantageously provides an interface via which the user controls operation of the tools.

Any of the methods described herein can be implemented by computer-executable instructions in (e.g., encoded on) one or more computer-readable media (e.g., computer-readable storage media or other tangible media). Such instructions can cause a computer to perform the method. The technologies described herein can be implemented in a variety of programming languages. Any of the methods described herein can be implemented by computer-executable instructions stored in one or more computer-readable storage devices (e.g., memory, magnetic storage, optical storage, or the like). Such instructions can cause a computer to perform the method.

### Example Computing System

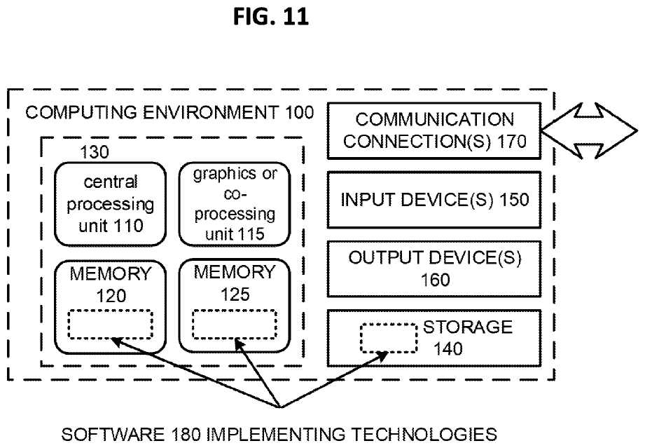

FIG. 11 illustrates a generalized example of a suitable computing system 100 in which several of the described innovations may be implemented. The computing system 100 is not intended to suggest any limitation as to scope of use or functionality, as the innovations may be implemented in diverse computing systems, including special-purpose computing systems. In practice, a computing system can comprise multiple networked instances of the illustrated computing system.

With reference to FIG. 11, the computing system 100 includes one or more processing units 110, 115 and memory 120, 125. In FIG. 11, this basic configuration 130 is included within a dashed line. The processing units 110, 115 execute computer-executable instructions. A processing unit can be a central processing unit (CPU), processor in an application-specific integrated circuit (ASIC), or any other type of processor. In a multi-processing system, multiple processing units execute computer-executable instructions to increase processing power. For example, FIG. 11 shows a central processing unit 110 as well as a graphics processing unit or co-processing unit 115. The tangible memory 120, 125 may be volatile memory (e.g., registers, cache, RAM), non-volatile memory (e.g., ROM, EEPROM, flash memory, etc.), or some combination of the two, accessible by the processing unit(s). The memory 120, 125 stores software 180 implementing one or more innovations described herein, in the form of computer-executable instructions suitable for execution by the processing unit(s).

A computing system may have additional features. For example, the computing system 100 includes storage 140, one or more input devices 150, one or more output devices 160, and one or more communication connections 170. An interconnection mechanism (not shown) such as a bus, controller, or network interconnects the components of the computing system 100. Typically, operating system software (not shown) provides an operating environment for other software executing in the computing system 2600, and coordinates activities of the components of the computing system 100.

The tangible storage 140 may be removable or non-removable, and includes magnetic disks, magnetic tapes or cassettes, CD-ROMs, DVDs, or any other medium which can be used to store information in a non-transitory way and which can be accessed within the computing system 100. The storage 140 stores instructions for the software 180 implementing one or more innovations described herein.

The input device(s) 150 may be a touch input device such as a keyboard, mouse, pen, or trackball, a voice input device, a scanning device, or another device that provides input to the computing system 100. For video encoding, the input device(s) 150 may be a camera, video card, TV tuner card, or similar device that accepts video input in analog or digital form, or a CD-ROM or CD-RW that reads video samples into the computing system 100. The output device(s) 160 may be a display, printer, speaker, CD-writer, or another device that provides output from the computing system 100.

The communication connection(s) 170 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

The innovations can be described in the general context of computer-executable instructions, such as those included in program modules, being executed in a computing system on a target real or virtual processor. Generally, program modules include routines, programs, libraries, objects, classes, components, data structures, etc. that perform particular tasks or implement particular abstract data types. The functionality of the program modules may be combined or split between program modules as desired in various embodiments. Computer-executable instructions for program modules may be executed within a local or distributed computing system.

For the sake of presentation, the detailed description uses terms like “determine” and “use” to describe computer operations in a computing system. These terms are high-level abstractions for operations performed by a computer, and should not be confused with acts performed by a human being. The actual computer operations corresponding to these terms vary depending on implementation.

### Computer-Readable Media

Any of the methods described herein can be implemented by computer-executable instructions in (e.g., stored on, encoded on, or the like) one or more computer-readable media (e.g., computer-readable storage media or other tangible media) or one or more computer-readable storage devices (e.g., memory, magnetic storage, optical storage, or the like). Such instructions can cause a computing device to perform the method. The technologies described herein can be implemented in a variety of programming languages.

Any of the computer-readable media herein can be non-transitory (e.g., volatile memory such as DRAM or SRAM, nonvolatile memory such as magnetic storage, optical storage, or the like) and/or tangible. Any of the storing actions described herein can be implemented by storing in one or more computer-readable media (e.g., computer-readable storage media or other tangible media). Any of the things (e.g., data created and used during implementation) described as stored can be stored in one or more computer-readable media (e.g., computer-readable storage media or other tangible media). Computer-readable media can be limited to implementations not consisting of a signal.

### IV. Additional Description of Embodiments of Interest

Clause 1. A method for classifying a type of cancer in a subject, comprising:

obtaining a plurality of sequence reads of a methylation sequencing assay covering genomic segments of a biological sample from a human subject with cancer, wherein the genomic segments contain the following genomic positions: chr8:102451058, chr19:16189360, chr2:114035619, chr10:5566908, chr16:678127, chr6:106958645, chr4:142054417, chr10:116064472, chr11:60619955, chr16:51184392, chr2:8724060, chr13:113424938, chr2:240270793, chr2:219256101, chr11:8284312, chr19:1827498, chr19:18335182, chr9:140683797, chr10:21788638, chr8:1895558, chr7:27196759, chr7:4801993, chr10:114591733, chr4:156588387, chr10:1120831, chr12:54427173, chr2:25600752, chr10:8097689, chr6:133562470, chr10:8097331, chr10:103603810, chr17:46655394, chr5:140306231, chr2:66665428, chr2:176994448, chr2:61372138, chr2:176994764, chr8:97506675, and chr17:46711341 according to a GRCh37/hg19 reference human genome;

assigning a methylation status of altered or normal to each of the genomic segments by comparing methylation of CpG sites of the sequence reads covering the respective genomic segments to a normal control; and

classifying the type of cancer in the subject into one of a plurality of different cancer types by comparing the methylation status of the genomic segments of the biological sample to a cancer type control, wherein the caner type control is the methylation status of the genomic segments in the different cancer types.

Clause 2. The method of Clause 1, wherein the different cancer types are colon cancer, rectum cancer, stomach cancer, pancreatic cancer, bladder cancer, head-neck cancer, lung cancer, breast cancer, kidney cancer, cervical cancer, liver cancer, prostate cancer, and uterine cancer.

Clause 3. The method of Clause 1 or Clause 2, wherein:

assigning a methylation status to the genomic segments containing chr10:8097331, chr10:8097689, chr10:103603810, chr10:116064472, chr11:8284312, chr12:54427173, chr16:51184392, chr17:46655394, chr17:46711341, chr19:16189360, chr19:18335182, chr2:8724060, chr2:61372138, chr2:66665428, chr2:114035619, chr2:176994448, chr2:176994764, chr4:142054417, chr4:156588387, chr5:140306231, chr6:106958645, chr6:133562470, chr7:27196759, and chr8:97506675 comprises calculating a ratio Y₁ according to:

Y₁=F₂/(F₁+F₂)

wherein F₁ and F₂ are frequencies of sequence reads in the plurality corresponding to a genomic segment where less than 40% or at least 60% of the CpG sites are methylated, respectively, and wherein a genomic segment is assigned an altered methylation status if there is an increase in the ratio Y₁ compared to the normal control and a genomic segment is assigned a normal methylation status if there is not an increase in the ratio Y₁ compared to the normal control; and

assigning a methylation status to the genomic segments containing chr10:1120831, chr10:5566908, chr10:21788638, chr10:114591733, chr11:60619955, chr13:113424938, chr16:678127, chr19:1827498, chr2:25600752, chr2:219256101, chr2:240270793, chr7:4801993, chr8:1895558, chr8:102451058, and chr9:140683797 comprises calculating a ratio Y₂ according to:

Y₂=F₁/(F₁±F₂)

wherein F₁ and F₂ are as defined above, and wherein a genomic segment is assigned an altered methylation status if there is an increase in the ratio Y₂ compared to the normal control and a genomic segment is assigned a normal methylation status if there is not an increase in the ratio Y₂ compared to the normal control.

Clause 4. The method of Clause 1 or Clause 2, wherein:

assigning a methylation status to the genomic segments containing chr10:8097331, chr10:8097689, chr10:103603810, chr10:116064472, chr11:8284312, chr12:54427173, chr16:51184392, chr17:46655394, chr17:46711341, chr19:16189360, chr19:18335182, chr2:8724060, chr2:61372138, chr2:66665428, chr2:114035619, chr2:176994448, chr2:176994764, chr4:142054417, chr4:156588387, chr5:140306231, chr6:106958645, chr6:133562470, chr7:27196759, and chr8:97506675 comprises calculating a ratio Y₃ according to:

Y₃=F₄/(F₃+F₄)

wherein F₃ and F₄ are frequencies of sequence reads in the plurality corresponding to a genomic segment where less than 20% or at least 80% of the CpG sites are methylated, respectively, and wherein a genomic segment is assigned an altered methylation status if there is an increase in the ratio Y₃ compared to the normal control and a genomic segment is assigned a normal methylation status if there is not an increase in the ratio Y₃ compared to the normal control; and

assigning the methylation status to the genomic segments containing chr10:1120831, chr10:5566908, chr10:21788638, chr10:114591733, chr11:60619955, chr13:113424938, chr16:678127, chr19:1827498, chr2:25600752, chr2:219256101, chr2:240270793, chr7:4801993, chr8:1895558, chr8:102451058, and chr9:140683797 comprises calculating a ratio Y₄ according to:

Y₄=F₃/(F₃+F₄)

wherein F₃ and F₄ are as defined above, and wherein a genomic segment is assigned an altered methylation status if there is an increase in the ratio Y₄ compared to the normal control and a genomic segment is assigned a normal methylation status if there is not an increase in the ratio Y₄ compared to the normal control.

Clause 5. The method of Clause 1 or Clause 2, wherein:

assigning a methylation status to the genomic segments containing chr10:8097331, chr10:8097689, chr10:103603810, chr10:116064472, chr11:8284312, chr12:54427173, chr16:51184392, chr17:46655394, chr17:46711341, chr19:16189360, chr19:18335182, chr2:8724060, chr2:61372138, chr2:66665428, chr2:114035619, chr2:176994448, chr2:176994764, chr4:142054417, chr4:156588387, chr5:140306231, chr6:106958645, chr6:133562470, chr7:27196759, and chr8:97506675 comprises calculating a ratio Y₅ according to:

Y₅=F₆/(F₅+F₆)

wherein F₃ and F₄ are frequencies of sequence reads in the plurality corresponding to a genomic segment where none or all of the CpG sites are methylated, respectively, and wherein a genomic segment is assigned an altered methylation status if there is an increase in the ratio Y₅ compared to the normal control and a genomic segment is assigned a normal methylation status if there is not an increase in the ratio Y₅ compared to the normal control; and

assigning the methylation status to the genomic segments containing chr10:1120831, chr10:5566908, chr10:21788638, chr10:114591733, chr11:60619955, chr13:113424938, chr16:678127, chr19:1827498, chr2:25600752, chr2:219256101, chr2:240270793, chr7:4801993, chr8:1895558, chr8:102451058, and chr9:140683797 comprises calculating a ratio Y₆ according to:

Y₆=F₅/(F₅+F₆)

wherein F₃ and F₄ are as defined above, and wherein a genomic segment is assigned an altered methylation status if there is an increase in the ratio Y₆ compared to the normal control and a genomic segment is assigned a normal methylation status if there is not an increase in the ratio Y₆ compared to the normal control.

Clause 6. The method of any one of Clauses 3-5, wherein the increase in the ratios Y₁ and Y₂, Y₃ and Y₄, and/or Y₅ and Y₆ compared to the normal control is an increase of at least 50%.

Clause 7. The method of any one of Clauses 3-5, wherein the increase in the ratios Y₁ and Y₂, Y₃ and Y₄, and/or Y₅ and Y₆ compared to the normal control is an increase of at least two standard deviations.

Clause 8. The method of any one of Clauses 1-7, wherein the genomic segments are plus or minus up to 300 bases of the genomic positions.

Clause 9. The method of any one of Clauses 1-8, wherein the genomic segments are plus or minus 50 to 300 bases of the genomic positions.

Clause 10. The method of any one of Clauses 1-9, wherein classifying the type of cancer in the subject into one of the plurality of different cancer types comprises comparing the methylation status of the genomic segments of the biological sample to the cancer type control using a distance calculation.

Clause 11. The method of any one of Clauses 1-10, wherein the methylation status of the genomic segments in the different cancer types of the cancer type control is as follows:

for colon and/or rectum cancer the genomic segments containing chr2:114035619, chr4:142054417, chr11:60619955, chr16:51184392, chr2:240270793, chr11:8284312, chr9:140683797, chr4:156588387, chr2:66665428, chr2:61372138, and chr8:97506675 have an altered methylation status and the remaining genomic segments have a normal methylation status;

for stomach cancer the genomic segments containing chr2:114035619, chr4:142054417, chr11:60619955, chr16:51184392, chr11:8284312, chr9:140683797, chr7:27196759, chr4:156588387, chr2:66665428, chr2:176994448, chr2:61372138, chr2:176994764, chr8:97506675, and chr17:46711341 have an altered methylation status and the remaining genomic segments have a normal methylation status;

for pancreatic cancer the genomic segments containing chr2:114035619, chr11:60619955, chr16:51184392, chr9:140683797, chr7:27196759, chr2:176994448, chr2:176994764, and chr17:46711341 have an altered methylation status and the remaining genomic segments have a normal methylation status;

for bladder cancer the genomic segments containing chr8:102451058, chr2:114035619, chr10:5566908, chr6:106958645, chr16:51184392, chr2:219256101, chr7:27196759, chr6:133562470, chr10:103603810, and chr5:140306231 have an altered methylation status and the remaining genomic segments have a normal methylation status;

for head-neck cancer if the genomic segments containing chr8:102451058, chr2:114035619, chr10:5566908, chr6:106958645, chr16:51184392, chr19:18335182, chr7:27196759, chr7:4801993, chr2:25600752, chr10:8097689, chr6:133562470, chr10:8097331, chr10:103603810, chr17:46655394, and chr5:140306231 have an altered methylation status and the remaining genomic segments have a normal methylation status;

for lung squamous cell carcinoma the genomic segments containing chr8:102451058, chr2:114035619, chr10:5566908, chr16:51184392, chr7:27196759, chr7:4801993, chr10:8097689, chr10:8097331, and chr17:46655394 have an altered methylation status and the remaining genomic segments have a normal methylation status;

for lung adenocarcinoma the genomic segments containing chr8:102451058, chr2:114035619, chr16:678127, chr16:51184392, chr13:113424938, chr7:27196759, and chr10:1120831 have an altered methylation status and the remaining genomic segments have a normal methylation status;

for breast cancer the genomic segments containing chr8:102451058, chr2:114035619, chr16:51184392, chr13:113424938, chr8:1895558, and chr7:27196759 have an altered methylation status and the remaining genomic segments have a normal methylation status;

for kidney cancer the genomic segments containing chr19:16189360, chr16:678127, chr11:60619955, chr19:1827498, chr9:140683797, chr10:21788638, and chr7:27196759 have an altered methylation status and the remaining genomic segments have a normal methylation status;

for cervical kidney renal papillary cell carcinoma the genomic segments containing chr19:16189360, chr11:60619955, chr9:140683797, chr10:21788638, chr7:27196759, and chr12:54427173 have an altered methylation status and the remaining genomic segments have a normal methylation status;

for liver cancer the genomic segments containing chr19:16189360, chr2:114035619, chr11:60619955, chr2:8724060, chr10:21788638, chr8:1895558, and chr7:27196759 have an altered methylation status and the remaining genomic segments have a normal methylation status;

for prostate cancer the genomic segments containing chr8:102451058, chr19:16189360, chr2:114035619, chr10:5566908, chr16:51184392, chr2:8724060, chr2:240270793, chr8:1895558, chr7:27196759, and chr10:114591733 have an altered methylation status and the remaining genomic segments have a normal methylation status; and/or

for uterine cancer the genomic segments containing chr8:102451058, chr13:113424938, chr10:21788638, and chr7:27196759 have an altered methylation status and the remaining genomic segments have a normal methylation status.

Clause 12. The method of any one of Clauses 1-11, wherein the cancer is classified as: colon and/or rectum cancer if the genomic segments containing chr2:114035619, chr4:142054417, chr11:60619955, chr16:51184392, chr2:240270793, chr11:8284312, chr9:140683797, chr4:156588387, chr2:66665428, chr2:61372138, and chr8:97506675 are assigned an altered methylation status and the remaining genomic segments are assigned a normal methylation status;

stomach cancer if the genomic segments containing chr2:114035619, chr4:142054417, chr11:60619955, chr16:51184392, chr11:8284312, chr9:140683797, chr7:27196759, chr4:156588387, chr2:66665428, chr2:176994448, chr2:61372138, chr2:176994764, chr8:97506675, and chr17:46711341 are assigned an altered methylation status and the remaining genomic segments are assigned a normal methylation status;

pancreatic cancer if the genomic segments containing chr2:114035619, chr11:60619955, chr16:51184392, chr9:140683797, chr7:27196759, chr2:176994448, chr2:176994764, and chr17:46711341 are assigned an altered methylation status and the remaining genomic segments are assigned a normal methylation status;

bladder cancer if the genomic segments containing chr8:102451058, chr2:114035619, chr10:5566908, chr6:106958645, chr16:51184392, chr2:219256101, chr7:27196759, chr6:133562470, chr10:103603810, and chr5:140306231 are assigned an altered methylation status and the remaining genomic segments are assigned a normal methylation status;

head-neck cancer if the genomic segments containing chr8:102451058, chr2:114035619, chr10:5566908, chr6:106958645, chr16:51184392, chr19:18335182, chr7:27196759, chr7:4801993, chr2:25600752, chr10:8097689, chr6:133562470, chr10:8097331, chr10:103603810, chr17:46655394, and chr5:140306231 are assigned an altered methylation status and the remaining genomic segments are assigned a normal methylation status;

lung squamous cell carcinoma if the genomic segments containing chr8:102451058, chr2:114035619, chr10:5566908, chr16:51184392, chr7:27196759, chr7:4801993, chr10:8097689, chr10:8097331, and chr17:46655394 are assigned an altered methylation status and the remaining genomic segments are assigned a normal methylation status;

lung adenocarcinoma if the genomic segments containing chr8:102451058, chr2:114035619, chr16:678127, chr16:51184392, chr13:113424938, chr7:27196759, and chr10:1120831 are assigned an altered methylation status and the remaining genomic segments are assigned a normal methylation status;

breast cancer if the genomic segments containing chr8:102451058, chr2:114035619, chr16:51184392, chr13:113424938, chr8:1895558, and chr7:27196759 are assigned an altered methylation status and the remaining genomic segments are assigned a normal methylation status;

kidney cancer if the genomic segments containing chr19:16189360, chr16:678127, chr11:60619955, chr19:1827498, chr9:140683797, chr10:21788638, and chr7:27196759 are assigned an altered methylation status and the remaining genomic segments are assigned a normal methylation status;

cervical kidney renal papillary cell carcinoma if the genomic segments containing chr19:16189360, chr11:60619955, chr9:140683797, chr10:21788638, chr7:27196759, and chr12:54427173 are assigned an altered methylation status and the remaining genomic segments are assigned a normal methylation status;

liver cancer if the genomic segments containing chr19:16189360, chr2:114035619, chr11:60619955, chr2:8724060, chr10:21788638, chr8:1895558, and chr7:27196759 are assigned an altered methylation status and the remaining genomic segments are assigned a normal methylation status;

prostate cancer if the genomic segments containing chr8:102451058, chr19:16189360, chr2:114035619, chr10:5566908, chr16:51184392, chr2:8724060, chr2:240270793, chr8:1895558, chr7:27196759, and chr10:114591733 are assigned an altered methylation status and the remaining genomic segments are assigned a normal methylation status; and/or

uterine cancer if the genomic segments containing chr8:102451058, chr13:113424938, chr10:21788638, and chr7:27196759 are assigned an altered methylation status and the remaining genomic segments are assigned a normal methylation status.

Clause 13. The method of any one of Clauses 1-12, wherein the methylation sequencing assay is a bisulfite sequencing assay.

Clause 14. The method of any one of Clauses 1-13, wherein the biological sample is a whole blood, serum, plasma, buccal epithelium, saliva, urine, stools, ascites, cervical pap smears, or bronchial aspirates sample.

Clause 15. The method of Clause 14, wherein the biological sample is a blood or plasma sample.

Clause 16. The method of any one of Clauses 1-15, wherein the biological sample contains cell-free DNA comprising the genomic segments.

Clause 17. The method of any one of Clauses 1-16, wherein the genomic segments are PCR amplified prior to sequencing.

Clause 18. The method of any one of Clauses 1-17, further comprising obtaining the biological sample from the subject.

Clause 19. The method of any one of Clauses 1-18, further comprising administering a therapeutically effective amount of an anti-cancer agent to the subject to treat the cancer in the subject.

Clause 20. The method of any one of Clauses 1-19, implemented at least in part using a computer.

Clause 21. A computing system, comprising:

one or more processors;

memory; and

a classification tool configured to:
receive a plurality of sequence reads of a methylation sequencing assay covering genomic segments of a biological sample from a human subject with cancer, wherein the genomic segments contain the following genomic positions: chr8:102451058, chr19:16189360, chr2:114035619, chr10:5566908, chr16:678127, chr6:106958645, chr4:142054417, chr10:116064472, chr11:60619955, chr16:51184392, chr2:8724060, chr13:113424938, chr2:240270793, chr2:219256101, chr11:8284312, chr19:1827498, chr19:18335182, chr9:140683797, chr10:21788638, chr8:1895558, chr7:27196759, chr7:4801993, chr10:114591733, chr4:156588387, chr10:1120831, chr12:54427173, chr2:25600752, chr10:8097689, chr6:133562470, chr10:8097331, chr10:103603810, chr17:46655394, chr5:140306231, chr2:66665428, chr2:176994448, chr2:61372138, chr2:176994764, chr8:97506675, and chr17:46711341 according to a GRCh37/hg19 reference human genome;
assign a methylation status of altered or normal to each of the genomic segments by comparing methylation of CpG sites of the sequence reads covering the respective genomic segments to a normal control; and
classify the type of cancer in the subject into one of a plurality of different cancer types by comparing the methylation status of the genomic segments of the biological sample to a cancer type control, wherein the caner type control is the methylation status of the genomic segments in the different cancer types.

### EXAMPLES

The following examples are provided to illustrate particular features of certain embodiments, but the scope of the claims is not limited to those features exemplified.

### Example 1

### Methods

### Data and Data Pre-Processing

To select markers/probes and analyze their performance, Infinium Human Methylation 450K BeadChip array data was used from 14 solid tumor types made available by TCGA (Table 1): bladder urothelial carcinoma (BLCA), breast invasive carcinoma (BRCA), colon adenocarcinoma (COAD), head and neck squamous cell carcinoma (HNSC), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), pancreatic adenocarcinoma (PAAD), prostate adenocarcinoma (PRAD), rectum adenocarcinoma (READ), stomach adenocarcinoma (STAD), and uterine corpus endometrioid carcinoma (UCEC).

TABLE 1
Blood reference and TCGA sample counts

| Blood reference | | | |
|---|---|---|---|
| or TCGA Cancer | | Normal | Tumor |
| Type | Description | (N) | (T) |
| ref | Peripheral blood | 2,711 | 0 |
| BLCA | Bladder urothelial carcinoma | 20 | 201 |
| BRCA | Breast invasive carcinoma | 96 | 676 |
| COAD | Colon adenocarcinoma | 38 | 274 |
| HNSC | Head & neck squamous cell | 50 | 426 |
| | carcinoma | | |
| KIRC | Kidney renal clear cell carcinoma | 160 | 296 |
| KIRP | Kidney renal papillary cell | 45 | 156 |
| | carcinoma | | |
| LIHC | Liver hepatocellular carcinoma | 50 | 151 |
| LUAD | Lung adenocarcinoma | 32 | 437 |
| LUSC | Lung squamous cell carcinoma | 42 | 359 |
| PAAD | Pancreatic adenocarcinoma | 9 | 65 |
| PRAD | Prostate adenocarcinoma | 49 | 248 |
| READ | Rectum adenocarcinoma | 7 | 96 |
| STAD | Stomach adenocarcinoma | 2 | 260 |
| UCEC | Uterine corpus endometrioid | 46 | 407 |
| | carcinoma | | |

For each type of cancer, data from both tumor and normal tissue were available; an ‘.N’ or ‘.T’ was appended to distinguish normal and tumor samples, respectively. The overwhelming majority of normal samples (621 of 646) were matched to tumor samples of the same type by participant id, indicating that these normal samples were from tissue adjacent to the cancer site. The remaining 25 normal samples (13 from UCEC, 6 from BRCA, 1 from LIHC, 3 from LUAD and 2 from LUSC) did not match any of the tumor samples used across all the types. In this analyses, colon adenocarcinoma (COAD) and rectum adenocarcinoma (READ) samples were pooled, resulting in a colorectal adenocarcinoma (CRAD) category, as the data were virtually indistinguishable in an initial analysis. Relevant to detecting tumors using blood plasma samples, GSE55763 samples were used as references for healthy blood DNA methylation levels [the Gene Expression Omnibus (GEO) repository, ncbi.nlm.nih.gov/geo]. GSE55763 dataset contains over 2,700 peripheral blood samples, with over 1,600 samples from healthy subjects with no reported condition/pathology, and over 1,000 samples from individuals with type 2 diabetes. The sample identities of the two categories were not available; however, 95% of all markers had methylation beta value standard deviation <0.07, indicating only negligible to small possible differences between the conditions for the overwhelming majority of markers. Given that, and the fact that in the marker selection algorithms markers with large standard deviation were filter out, it was concluded that the presence of ˜40% of blood samples from individuals with type 2 diabetes in this dataset does not preclude its use as a healthy peripheral blood reference (PB_{ref}) for purposes herein. Thus, in total data from 13 tumor types were analyzed, plus the PB_{ref} samples (Table 1).

To validate the performance of the selected probes, the following GEO methylation array datasets were used: GSE37754, GSE49149, GSE53051, GSE55479, GSE61441, GSE66695, GSE69914. Whole genome bisulfite sequencing (WGBS) data from Chan et al. (PNAS, 110(47):18761-18768, 2013) was also used (Table 2). Additionally, methylation arrays for several non-cancer conditions were used to serve as negative controls: GSE32148, GSE85566, GSE50874, GSE49542, and GSE87621, as well as array data from Dayeh et al. (2014) (see ludc.med.lu.se/research-units/epigenetics-and-diabetes/published-data/dna-methylation-human-islets/) (Table 3).

TABLE 2
Validation samples: tumors and normal controls

| | | Sample | |
|---|---|---|---|
| Data Source | Description | Count | Format |
| Chan et al. | solid HCC | 15 | WGBS |
| (PNAS, | normal plasma | 32 | WGBS |
| 110(47): 18761- | | | |
| 18768, 2013) | | | |
| GSE49149 | pancreatic ductal | 167 | Infinium 450K array |
| | adenocarcinoma | | |
| | adjacent non-tumor | 29 | Infinium 450K array |
| GSE37754 | breast tumor | 62 | Infinium 450K array |
| | adjacent noncancerous | 10 | Infinium 450K array |
| | tissue | | |
| GSE66695 | breast tumor | 80 | Infinium 450K array |
| | normal tissue | 40 | Infinium 450K array |
| GSE69914 | breast cancer | 305 | Infinium 450K array |
| | cancer-brca1 | 3 | Infinium 450K array |
| | Normal | 50 | Infinium 450K array |
| | normal adjacent | 42 | Infinium 450K array |
| | normal-brca1 | 7 | Infinium 450K array |
| GSE61441 | clear cell renal cell | 46 | Infinium 450K array |
| | carcinoma | | |
| | matched normal | 46 | Infinium 450K array |
| | kidney tissue | | |
| GSE53051 | breast cancer | 14 | Infinium 450K array |
| | breast normal | 10 | Infinium 450K array |
| | colon cancer | 35 | Infinium 450K array |
| | colon normal | 18 | Infinium 450K array |
| | lung cancer | 9 | Infinium 450K array |
| | lung normal | 11 | Infinium 450K array |
| | pancreas cancer | 29 | Infinium 450K array |
| | pancreas normal | 12 | Infinium 450K array |
| GSE55479 | prostate cancer | 143 | Infinium 450K array |

To prepare the array data, Illumina Infinium array beta methylation values were BMIQ-normalized. To align the WGBS data and extract methylation information, samtools, bcftools, and bismark were used. Perl and R were used for downstream data processing. The hg19 human reference genome, and the Illumina annotation file of the Illumina Infinium HumanMethylation450 Beadchip array were used to obtain information on probes.

### Marker Selection Strategy

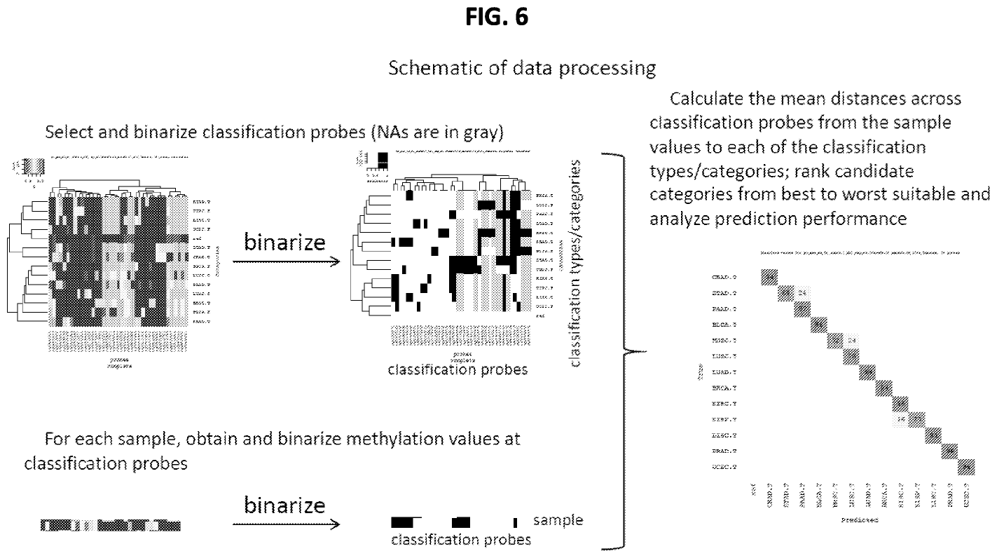

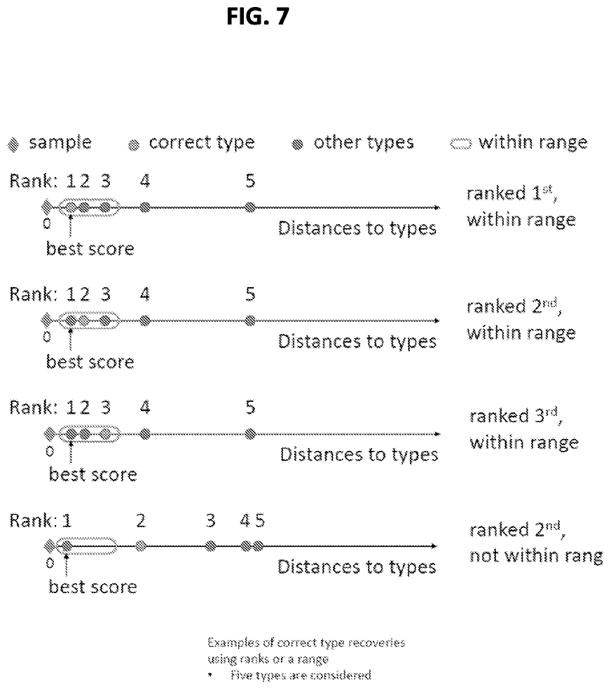

Since, in the blood-based diagnostics, it was expected that the tumor signal would be diluted in the normal blood background, loci whose normal methylation represented the extremes of the scale were assessed, i.e., virtually absent or saturated, with minor variability across normal reference samples. In such cases, a weakly abnormal signal becomes apparent as an outlier against the background. In addition, binary calls were made at each CpG position to describe whether it differs from the normal state or not, permitting indeterminate calls as well. To enhance robustness of the classification, several independent sets of markers were selected, with redundancy between them, wherein each set was either selected to independently classify a sample by type or to distinguish tumor from normal sample. These marker sets were pooled into corresponding combined panels for the scoring analysis, to enhance performance. The maximum number of total probes was capped at 48 for compatibility with current experimental platforms, as described next. The technical details of marker selection and subsequent utilization for tumor type classification and tumor detection are provided below (see also FIGS. 6-7).

### Bisulfite Amplicon Sequencing

To further validate the markers in the panels, their performance was tested using bisulfite amplicon sequencing. To do so, DNA was obtained from three sources. First, 13 DNA samples of normal blood plasma (1 μg DNA each) were purchased from Fox Chase Cancer Center. This DNA had been extracted using Qiagen Mini-prep kits or the Qiagen Autopure. Second, tumor and normal DNA samples (5 μg DNA each) were collected from Origene for colon, stomach, pancreas, lung, breast, kidney, liver and prostate tissue. There were 5 tumor samples for each tissue, except stomach with 4 samples, and there were 3 normal samples for each tissue, except colon and liver with 2 samples each. DNA was isolated in a proprietary protocol similar to the EasyDNA isolation system provided by Invitrogen. Finally, 5 tumor and 2 normal 1-μg DNA samples were collected from BioServe for each of the following types of tissue: breast, stomach, and lung. This DNA had been isolated by grinding the tissue under liquid nitrogen, lysing it overnight with SDS buffer and proteinase K, subjecting it to RNaseA treatment, and precipitating the DNA. In the analysis, all these samples were have excluded as they had 5 times less DNA than colon and liver samples from Origene; sequencing data quality was a serious concern, with missing or unreliable measurements, especially in lung samples.

DNA was processed and sequenced as described below. 500 ng per sample was used for bisulfite conversions. After bisulfite conversion, 50 ng of the bisulfite converted DNA was input the Fluidigm Access Array system. This translates to roughly 1 ng per primer set.

Assays were designed to target CpG sites in the specified regions of interest (ROIs) using custom primers created for this purpose. Parameters were selected such that PCR amplicons would be 100 to 300 bp. In addition, as much as possible, primers were designed that would not anneal to CpG sites in the ROI. In the event that CpG sites were absolutely necessary for target amplification, primers were ordered to be synthesized with a pyrimidine (C or T) at the CpG cytosine in the forward primer, or a purine (A or G) in the reverse primer to minimize amplification bias due to either a methylated or unmethylated allele.

All primers were resuspended or ordered in TE solution at 100 μM. Primers were then mixed (if necessary) and diluted to 2 μM each. Primers were tested using real-time PCR with 1 ng bisulfite-converted control DNA, in duplicate individual reactions. DNA melt analysis was performed to confirm the presence of a specific PCR product. The following guidelines were used to assess performance (i) had average crossing point (Cp) values <40, (ii) duplicate Cps did not have a Cp difference >1 (within 5% CV), (iii) reached the plateau phase before the run ended at cycle 45, (iv) produced melting curves in the expected range for PCR products, and (v) duplicate melts had calculated melting temperatures within 10% CV.

Following primer validation, 25 samples (5 each) of breast, colon, liver, lung, and stomach tumor; 2 normal samples of each of these tissue types; and 13 normal blood plasma samples were bisulfite converted using the EZ DNA Methylation-Lightning™ Kit (www.zymoresearch.com-catalog number D5030), at 500 ng a sample and according to the manufacturer's instructions. Multiplex amplification of all samples was performed according to the manufacturer's instructions, using 50 ng bisulfite converted DNA (roughly 1 ng per primer set), ROI-specific primer pairs, and the Fluidigm Access Array™ System. After barcoding, samples were purified (ZR-96 DNA Clean & Concentrator™-ZR, Cat #D4023) and then prepared for massively parallel sequencing using an Illumina MiSeq V2 300 bp Reagent Kit and paired-end sequencing protocol according to the manufacturer's guidelines. Sequencing read data were aligned using bismark and extracted as uniquely aligning reads across the 46 amplicons. To calculate methylation at each locus, amplicon-wide averages across multiple CpGs in each amplicon were used to reduce uncertainty due to data quality concerns.

### Plasma WGBS Analysis

In the comparison of plasma samples from 32 controls and from 26 HCC subjects (Chan et al., PNAS, 110(47):18761-18768, 2013), for each sample and at each of the 46 probe loci sequenced reads overlapping the respective genomic intervals within 200 bp from the probe CpG position were extracted.

The most straightforward calculation is to average methylation of all sequenced CpGs in the region (here, reads overlapping an interval within 200 bp from the probe). Thus, unmethylated and methylated CpGs across all the individual reads were separately added up and the counts denoted as cu and cm, respectively.

Other alternatives include considering only fully methylated and fully unmethylated reads. The rationale behind these alternatives is based on our previous studies of ZNF154 promoter locus (Sanchez-Vega et al., Epigenetics, 8(12):1355-1372, 2013; Margolin et al., J Mol Diagn., 18(2):283-298, 2016) where focus on individual reads/fragments with either zero or full methylation of multiple CpGs resulted in improved classification performance under signal dilution simulations, compared to using average methylation. It was also desired to give more weight to reads containing more CpGs (either all methylated or all unmethylated), as a soft thresholding alternative to considering only reads with a certain minimal number of CpGs.

Here, cu and cm are defined as (weighted) counts/numbers of fully unmethylated and fully methylated reads, respectively. Two functional forms for weights were considered: (i) number of CpGs in a corresponding read, N, raised to some power, r, (i.e., N^{r}), with r=0, 1 or 2 (note that r=0 means unweighted read counts), and (ii) some base, b, raised to the power of number of CpGs (i.e., b^{N}), with b=2.

Next, given the values of cu and cm, the signal fraction x was calculated for each sample at each locus. Based on the blood reference level of methylation at the probe, x=cm/(cu+cm) was either calculated when reference is close to 0, or x=cu/(cu+cm) when reference is close to 1.

Since tumor signal in plasma is diluted, binarization thresholds at each probe locus were simply selected as the highest observed x-value in the 32 control plasma samples. If a given sample had an x-value above threshold it was set as 1, or otherwise it was set to zero. After binarization, tumor-normal calling and tumor type classification was performed as described below.

Two tests were used to verify that tumor methylation signal at the probe loci expected to show signal relative to the reference for HCC (LIHC.T) is stronger than at the loci expected to be similar to the reference. This defines two probe/locus groups: one with binarized LIHC.T class probe values of 1 and the other with values of 0 (and the NA values were ignored). First, for each of the alternative calculations of x, a one-sided Spearman correlation test was performed between the 39 binarized LIHC.T class probe values and the corresponding locus averages across the HCC plasma samples. Second, a one-sided Wilcoxon test on these locus averages between the two probe groups was performed.

### Example 2

### Tumor Type Classification

### Selection of Tumor-Type Classification Probes

A pool of candidate probes was preselected to assign tumor samples one of the TCGA types considered, or blood reference, drawing from all of the probes/markers present on the 450 k Illumina methylation arrays. During the selection process, several criteria were employed:
(1) Each locus had to have at least four CpGs within 50 bp of the probe (including itself);
(2) Each probe had to be predominantly unmethylated or methylated in blood reference (median beta value <0.15 or >0.85, with standard deviation <0.15), with less than 3.7% missing data (100 values out of 2,711 samples); and
(3) Each probe had to have a substantially different methylation level from the blood reference in at least one tumor type (i.e., median >0.35 when reference methylation was near zero, or median <0.65 when reference methylation was near 1.0), while the probe's methylation in all remaining tumor types had to satisfy the same thresholds as were valid for the reference.

This resulted in 2,130 candidate probes. Additional filtering was then applied, keeping only probes whose target CpGs had average beta values either <0.1 or >0.9 in the WGBS sequencing of 32 control blood plasma samples reported by Chan et al. (PNAS, 110(47):18761-18768, 2013) (samples were pooled for the calculation of average). This reduced the candidate pool to 1,220 loci.

Tumor-type classification markers were selected from this pool of 1,220 candidate probes. First, median beta values of candidate loci were binarized. Specifically, blood reference median values were rounded to either 0 or 1. If a probe had a rounded reference value of 0 in peripheral blood, then for each of the classification categories/types (i.e., the tumor types considered, as well as the blood reference itself), the binarized value was set to 1 if the median for that type was >0.35, or set to 0 if the median was ≤0.15, or otherwise NA (not available). Similarly, if a reference locus had a rounded value of 1, then for each of the classification types, the binarized value was set to 1 if the median value for that type was <0.65, or set to 0 if the median was 0.85, or NA. In this way, the binarized reference values all end up being set to 0, both for probes with low and with high methylation. Values of 1 in other classification types indicate that the methylation is sufficiently far from reference (as defined by the thresholds), while values of 0 indicate methylation similar to the reference.

The binarized candidate marker values were used to iteratively choose the set of tumor-type classification markers. In each step, a marker with maximal entropy across all subsets of classification types was selected, as described in the following. A subset of classification types was called ambiguous if it had more than one type. We started with an initial single (sub)set of all types together (14 classification types: PB_{ref}, CRAD.T, STAD.T, PAAD.T, BLCA.T, HNSC.T, LUSC.T, LUAD.T, BRCA.T, KIRC.T, KIRP.T, LIHC.T, PRAD.T and UCEC.T). In each iteration step, for each probe/marker in each (ambiguous) subset, the entropy was calculated as −nΣᵢ₌₀¹pᵢlogpᵢ, where pᵢ are the fractions of i's (0's and 1's) in the binarized probe values and n is the subset cardinality. In the case of NAs in a subset, the entropy was set to zero. The entropies across the subsets were then added up for each marker, with the intent of choosing a marker with the maximal sum. (When there were multiple markers with an identical entropy sum, the first one with smallest Euclidean distance between its median beta values and its binarized values (or their reciprocal, i.e., one minus binarized values) was chosen, across the types in ambiguous subsets.) Given the marker, the subsets in which it has both 0's and 1's (and no NAs) are split in two per these values, and this marker is excluded from subsequent iterations. If, after splitting, a new subset contains single classification type, this subset (or type) is no longer ambiguous and is excluded from further iterations. If there are no probes with positive entropy the process stops due to failure; whereas the process stops successfully if there are no ambiguous subsets left to split.

The algorithm for selecting classification markers can be run multiple times, by excluding previously selected markers and possibly also markers in the genomic neighborhood, to obtain new sets of classification markers. Three sets of markers (each successfully splitting the classification types) were initially compiled, and candidates were excluded within 100 bp of each selected marker. The three sets together yielded 27 markers.

When sample type classification was performed (see next section) using these 27 markers, some tumor types were predicted worse than others. To improve the ability to assign samples to the correct type, the selection algorithm was applied separately to each of the two worst clusters of BLCA-HNSC-LUSC and CRAD-STAD-PAAD tumors, ignoring all other tumor types except the reference. Additionally, each marker was required to have at least one tumor type satisfying the thresholds attained for the reference (in view of the goal to distinguish between the types, not distinguish between tumor types and reference). This yielded 273 and 1,684 candidate loci, respectively. After triple runs of the algorithm, an additional six probes were added for each cluster, raising the total number of classification probes to 39, based on the following genomic positions (GRCh37/hg19): chr10:1120831, chr10:5566908, chr10:21788638, chr10:114591733, chr11:60619955, chr13:113424938, chr16:678127, chr19:1827498, chr2:25600752, chr2:219256101, chr2:240270793, chr7:4801993, chr8:1895558, chr8:102451058, chr9:140683797, chr10:8097331, chr10:8097689, chr10:103603810, chr10:116064472, chr11:8284312, chr12:54427173, chr16:51184392, chr17:46655394, chr17:46711341, chr19:16189360, chr19:18335182, chr2:8724060, chr2:61372138, chr2:66665428, chr2:114035619, chr2:176994448, chr2:176994764, chr4:142054417, chr4:156588387, chr5:140306231, chr6:106958645, chr6:133562470, chr7:27196759, and chr8:97506675.

In normal tissue, CpG sites located at chr10:1120831, chr10:5566908, chr10:21788638, chr10:114591733, chr11:60619955, chr13:113424938, chr16:678127, chr19:1827498, chr2:25600752, chr2:219256101, chr2:240270793, chr7:4801993, chr8:1895558, chr8:102451058, and chr9:140683797 are methylated, and CpG sites located at chr10:8097331, chr10:8097689, chr10:103603810, chr10:116064472, chr11:8284312, chr12:54427173, chr16:51184392, chr17:46655394, chr17:46711341, chr19:16189360, chr19:18335182, chr2:8724060, chr2:61372138, chr2:66665428, chr2:114035619, chr2:176994448, chr2:176994764, chr4:142054417, chr4:156588387, chr5:140306231, chr6:106958645, chr6:133562470, chr7:27196759, and chr8:97506675 are not methylated.

The methylation beta values and binarized values of the added probes were set to NA for the types ignored in their selection (FIGS. 6-7).

### Sample Type Classification

### Distance to Classification Type

The central classification measurement for each sample described is mean distance, described as follows. Each classification type is represented by values of the 39 classification markers, and each sample has methylation beta values at those positions (since WGBS is not deep the methylation signal within 50 bases from the probe coordinate was averaged; similarly, amplicon-wide averages were used in the targeted sequencing, to reduce uncertainty due to data quality concerns). Binarized median beta values were used for the classification types, as well as binarized beta values of individual samples (using the same thresholds as during marker selection). Note that binarization of individual samples can introduce NAs even when the corresponding classification type marker values are available (i.e., 0 or 1). The arithmetic mean (instead of sum) of all non-NA squared differences between the sample and the classification marker values was used, in line with Euclidean distance calculation. Taking the mean of non-NA values compensates for the possible difference in number of NAs in different samples. Note that for the binarized data, taking the square of the value is identical to simply taking the absolute value, as the only possible numbers are 0's and 1's.

Thus, for each sample there is vector of mean distances to the classification types. The simplest classification is to the closest type (for most samples, the best predicted type is unique; in case of tied types sample contribution is split uniformly among the tied types yielding expected classification performance with randomly resolved ties). This worked well and is reported, unless stated otherwise; several extensions were also considered, as well as additional descriptors of the classification performance beyond the best pick.

### Distance Adjustments

Two ways to adjust for the classification bias due to different distributions of distances from individual samples to the correct classification type in different types were considered. In the first approach, each sample's quantile fraction in each classification type was calculated, i.e., the fraction of samples of that type with distances less than the one observed (plus half the fraction of samples having distances exactly equal to the one considered). The result for each sample was a vector of sample quantile fractions in the classification types, and the simplest classification would be a type with the smallest fraction. For these calculations (which are calculations of empirical cumulative distribution functions), both the raw distributions (i.e., collections of calculated distance values), as well as their parametrizations (see fitted distributions section, below) were used.

In the second approach, naïve Bayes approximation was used to estimate posterior probabilities for a sample to belong to each of considered 14 (classification) types, given the vector of distances. Using Bayes' formula, this probability is given by

$$P ( i \vert \{ {d}_{j} \} ) = \frac{P ( \{ {d}_{j} \} \vert i ) P ( i )}{{\sum }_{k} P ( \{ {d}_{j} \} \vert k ) P ( k )} ,$$

where {dⱼ} is a vector of distances of a given sample to all the classification types, i is the considered/possible resulting type in sample classification, and k runs through all possible classification types. P(i) are prior probabilities and are taken to be identical in this work; however, they can be adjusted to the observed prevalence of different types of tumor. Naïve Bayes approach approximates P({dⱼ}|k) with ΠⱼP(dⱼ|k); hence, only the univariate distributions of distances from samples of type k to classification type j, for all possible values of j and k, needed to be known. The raw (observed) distributions were fitted as described below, and P(dⱼ|k) was calculated by integrating the fitted densities in a small interval (0.01) around dⱼ. In most cases, one could use densities instead of probabilities, as Bayes' formula only contains ratios; however, for an exact zero distance, integration will always yield a non-zero value (even without the point mass at 0, as discussed in the next section), thus allowing for a non-zero estimate for any valid P({dⱼ}|k). The result of this approach is then a vector of estimated posterior probabilities of possible sample types, and the simplest classification would be a type with highest probability.

### Fitted Distributions of Sample Distances to Classification Types

Raw distributions of sample distances to classification types were approximated by beta distributions, with a modification. Due to binarization, there is a noticeable number of exact zero distances. In order to reflect that, finite masses at the extreme distance values of 0 and 1 were allowed (the point mass at 1 was added for symmetry, and its actual mass always was 0). After assigning the distances of exact 0 and 1 to the point masses, the remainder were fit using R function fitdistr from package MASS. Optionally, the fitted distributions of the TCGA normal tissue types (.N) were also combined together with blood reference distributions, by equally weighting them and using the law of total variance (the point masses at 0 and 1 were not used here). This option was used when calculating fitted quantile fractions, resulting in a substantial increase in the proportion of normal TCGA samples classified as reference, with a smaller effect on TCGA tumors (classification using QFfit).

### Classification Evaluation

Thus far, several different measures have been described that can be calculated for a sample of interest for classification: a vector of distances to possible classes/outcomes/classification types, vectors of quantile fractions (both raw and fitted), and a vector of probabilities. Note that all these values are defined to lie in the interval [0,1]. Here, these measures were not combined to improve the overall performance; the classification performances based on measures other than ranked distances is reported. The simplest classification using any of these measures was to choose the best class (shortest distance, highest probability, etc.). However, from a practical perspective, it would be desirable to establish criteria for how reliable the classification results are for each individual sample and when to consider the second-best and other possibilities. To this end, several statistics were considered for each sample. To estimate the classification performance on the samples of known type the following were performed: (1) check whether the best class was correct (i.e., whether it matched the sample's known type—this is the simplest classification), (2) check whether any of the classes ranked (i.e., best or second-best, with nuances in case of ties) were correct, and recorded the rank of the correct class, and (3) define ranges within which the class measures could be accepted (irrespective of rank), checked whether the correct class was within range, and recorded the number of classes within range. The ranges were defined as follows: distance up to 1.1*max{shortest distance, 0.1}, quantile fraction up to 0.9 and up to 0.95 (i.e., 90% and 95%), and posterior probability ≥0.1.

Classification with Random Forests

The R randomForest package was used to perform random forest classifications on binarized data. Default parameters were used, unless stated explicitly otherwise. The list of 3,077 candidate classification probes was obtained after merging separate lists for the 1,220 initial candidate probes with 273 and 1,684 candidate probes for subsequent refinements. An alternative subset of probes, derived from this set of 3,077 probes, was selected by only retaining probes with high (>7) maximal importance in at least one type/class. The class-wise importances are provided by the algorithm output.

### Example 3

### Tumor-Normal Calling

### Selection of Tumor-Normal Calling Markers

To make a pool of candidate tumor-normal (T-N) markers, the first two criteria were identical to the selection of candidate tumor-type classification probes above: (1) require at least four CpGs within 50 bp of the probe and (2) each probe had to be predominantly unmethylated or methylated in blood reference. Additionally, it was required that (3) each marker had to differ substantially from the blood reference in at least one tumor type (median methylation beta values of tumor samples of that type >0.4 when reference methylation was near zero, or median <0.6 when reference methylation was near 1). There were no conditions imposed on the remaining tumor types; however, median methylation was set to NA for any tumor type (and its normal counterpart) violating the same thresholds as were valid for the reference. Lastly, (4) it was required that all normal tissue types were similar to the reference (normal samples of each of the 13 types satisfied the same thresholds as the blood reference, allowing up to 50% missing values per type). This yielded 4,287 candidate markers, with accompanying median methylation values, or NAs.

From this pool of candidate markers, T-N calling markers were selected. The analysis was started with a list of all 13 tumor types, which initially remain unresolved. At each iteration, a probe was chosen whose median methylation was substantially different from median methylation in blood reference samples (as defined above), in the maximal number of remaining tumor types. (In case of multiple such probes the first one with the maximal absolute difference between the mean methylation across the remaining types in tumors (excluding NAs) and the reference was chosen.) After the probe was selected, tumor types that were substantially different from reference, were thus resolved and removed from the list of remaining tumor types. Then the chosen marker, as well as all of its neighbors within 100 bp were excluded from subsequent iterations. Iterations proceeded until no tumor types remained, or until the approach failed to find suitable markers. This algorithm can be run multiple times and selected new markers when previous selections (and their neighbors) are excluded from consideration. Markers from two runs were selected, with each set of markers resolving all of the types, and together the two sets yielded 8 T-N call probes. These 8 probes were called “unconditional” T-N call probes, because within the collection of 13 tumor types considered here, these probes can be used to differentiate tumors of all types from normal samples and PB_{ref}, without knowing the type a priori. One of the 8 probes was also present among the 39 tumor-type classification probes discussed above, thus giving a total of 46 unique probes.

The 8 T-N call probes are based on the following genomic positions (GRCh37/hg19): chr17:40333009, chr17:46655394, chr6:88876741, chr6:150286508, chr7:19157193, chr10:14816201, chr12:129822259, and chr14:89628169.

In normal tissue, CpG sites located at chr10:14816201, chr12:129822259, and chr14:89628169 are methylated and CpG sites located at chr17:40333009, chr17:46655394, chr6:88876741, chr6:150286508, and chr7:19157193 are not methylated.

### Sample Calling

For each sample, methylation beta values were binarized at the T-N markers. Here, the thresholds for setting the value to 1 were >0.4 for markers with low reference methylation, and <0.6 for markers with high reference methylation, in agreement with probe selection thresholds. A sample was called as tumor if at least one of the binarized values was 1; otherwise, it was called as normal.

### Modification of Type Classification by Tumor-Normal Calls

When the two prediction steps were combined (tumor-type classification and T-N calling), the best prediction class for a sample was changed to reference if the T-N call was normal.

### Addressing Overfitting in TCGA and PB_{ref} Data

Generally speaking, designing a classifier and estimating its performance on the same dataset might lead to overfitting (with overoptimistic performance estimates). However, one should not expect noticeable overfitting in our tumor-type classification and T-N calling, as the criteria are primarily based on median methylation values, which are simple, stable and limited summaries of the data. Using leave-one-sample-out cross-validation, only medians of that sample's type were (marginally) affected, if at all, in each round. For an explicit calculation, samples of each type were split into training (90%) and validation (10%) sets, and used the training set for probe selection. For the T-N calling, true positive rates were similar in each set (93.7% and 95.1% respectively; both at 95.1% if weighted by number of samples in each type), compared to a lower 90.3% (91.4%, weighted) using the markers derived from all the data and reported in the Results section. However, the false positive rates for TCGA normal tissues were also higher, at 3.6% and 7.5%, respectively (3.6% and 7.6%, weighted), compared with 1.3% (1.2%, weighted) using the markers derived from all of the data. In addition, one of the PB_{ref} training samples (out of 2,440) was miscalled as a tumor. The increased false positive rate in validation samples was due, in large part, to two normal PRAD samples, which were consistently called tumors in multiple scenarios and coincidently ended up in the validation set. In tumor-type classification, training and validation sets yielded 84.6% and 84.0% (weighted 85.1% and 84.9%) correct, respectively (best by distance), compared to 85.3% (weighted 86.1%) using the probes derived from all the data and reported in the Results section. None of the training and two of the validation set PB_{ref} samples were classified incorrectly (as PAAD.T); however, combination of T-N calling and tumor-type classification leads to correct prediction for all reference samples. It is concluded that type classification and T-N calling performances are comparable between the training and validation sets.

### Example 4

### Performance of 39-Marker Tumor-Type Classification Panel

This example illustrates the identification of a number of methylation sites capable of distinguishing amongst the 13 major tumor types studied by TCGA and healthy peripheral blood (Table 1). nUsing DNA methylation data from 4,052 samples from 13 tumor types and 2,711 peripheral blood reference samples (Table 1), 39 CpG loci were identified that could be used for tumor-type classification (see Methods).

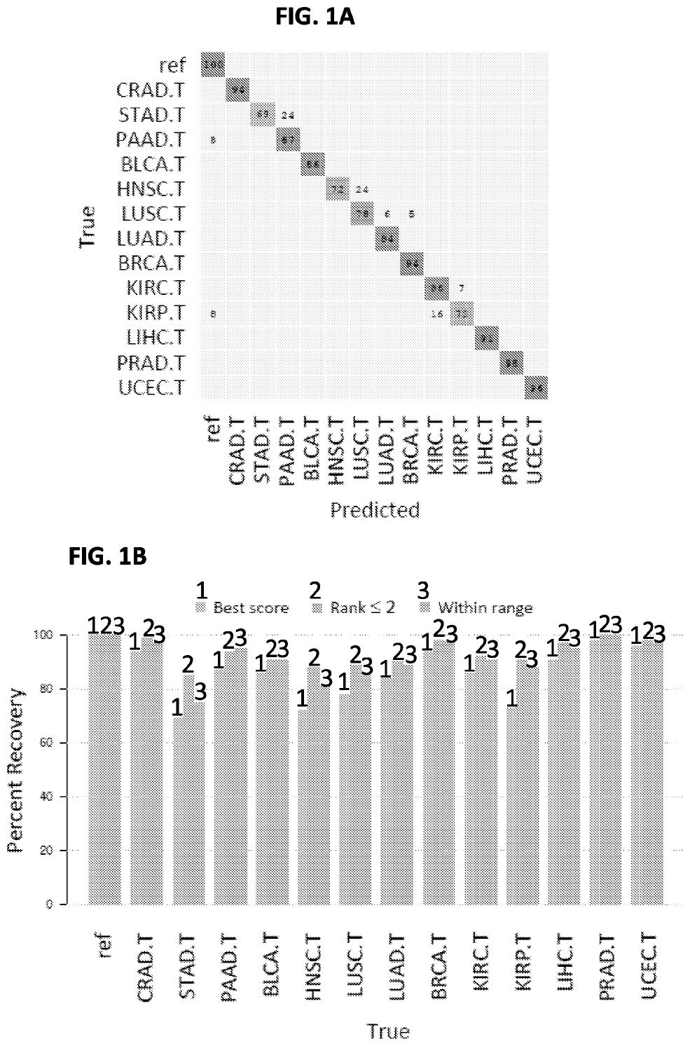

When applied to the discovery dataset, the 39-marker classification panel returned a median of 86% correct classifications across all 13 tumor types (range 69-98%) (FIG. 1A). Five tumor types (colorectal, breast, liver, prostate, and uterine) returned >90% correct classifications. Four additional tumor types (pancreas, bladder, lung adenocarcinoma and kidney renal cell) were classified correctly 84 to 87% of the time, whereas the four remaining tumor types (stomach, head and neck, lung squamous cell and kidney papillary renal) were classified correctly 69 to 78% of the time. In most cases, the samples misclassified as either another cancer type from the same organ (in the case of lung adenocarcinoma and lung squamous cell or kidney renal cell and kidney papillary adenocarcinoma), or a cancer from an adjacent organ (in the case of stomach and pancreas tumors). The exception was head and neck tumors, where 24% classified as lung squamous cell tumors; however, previous studies have found that lung squamous, head and neck squamous, and a subset of bladder adenocarcinomas coalesce into one subtype (Hoadley et al., Cell, 158(4):929-944, 2014). Finally, 99.9% of the 2,711 peripheral blood reference samples were classified correctly as reference samples (only two samples were incorrectly classified as pancreas tumors). These findings indicate that the marker panel robustly distinguishes among the 13 tumor types when used on DNA methylation data extracted directly from tumors.

### Additional Classification Criteria/Characterization

To gain further insights into the classification performance two alternative modifications of the analysis that increase correct tumor type recovery percentage were identified (FIG. 1B). Specifically, in the initial approach i.e., “best match” the best scoring match was picked after comparison of a sample to every tumor-type and the blood reference category. Because the site of origin is known, it is possible to assess the classification accuracy (Methods; FIG. 7). If, in addition to the best match, all predicted types ranked second or better were included in each sample classification, i.e., “rank ≤2” the fraction of samples with recovered correct type rises to 93% (range 85-100%) (FIG. 1B). Alternatively, if, in addition to the best match, certain range of score values are accepted, i.e., “within range” and include those respective types, the average rate of recovering the correct type is 91% (range 75-100%) (FIG. 1B).

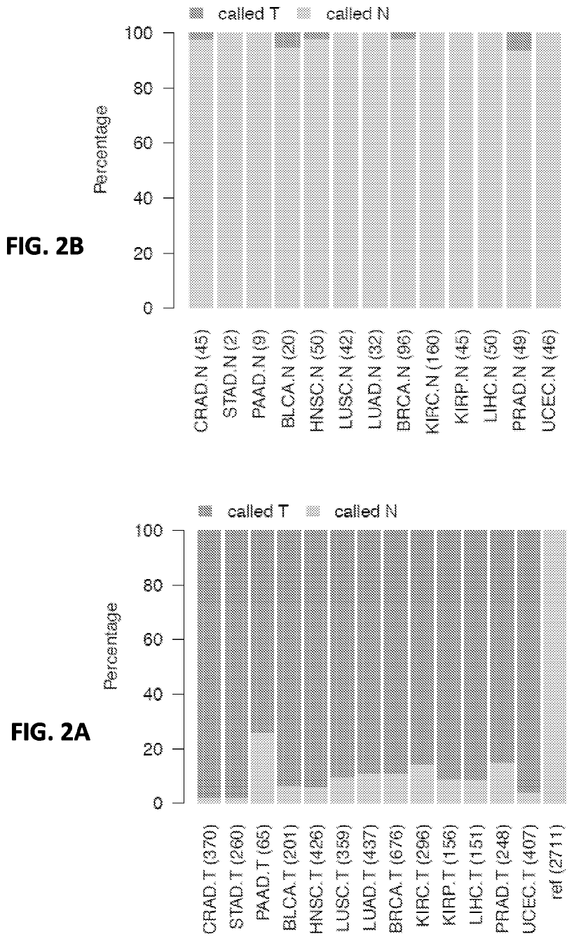

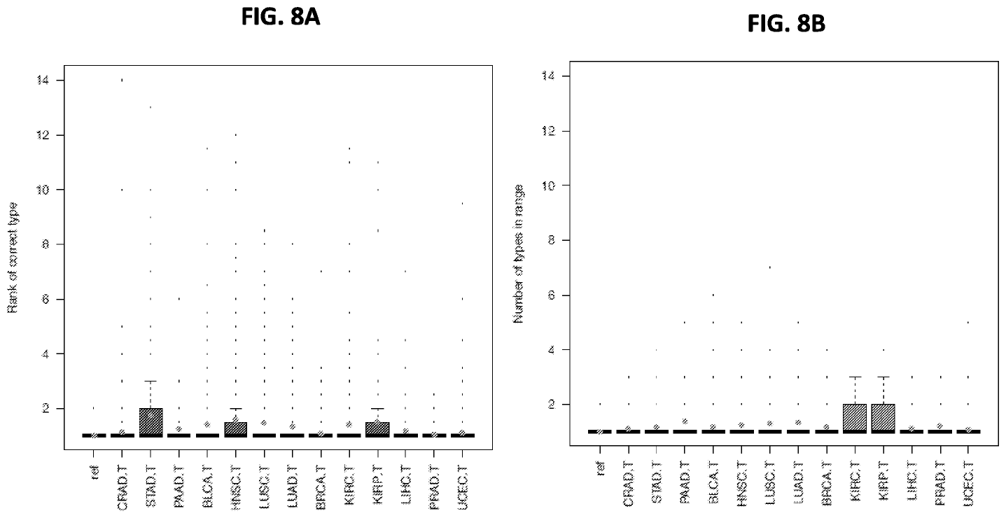

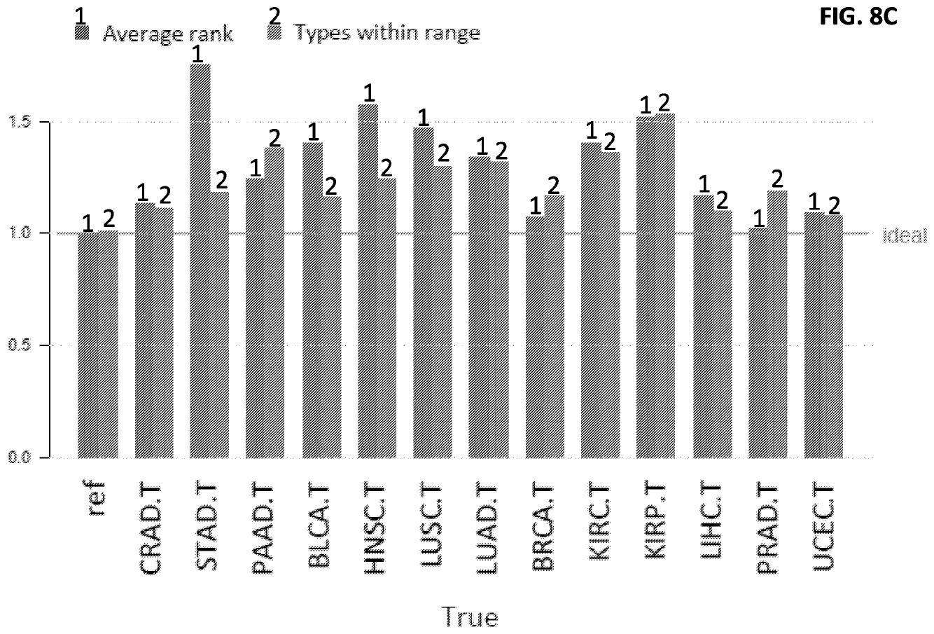

These two approaches recovered the correct type more often than by considering the best match predictions alone (FIG. 2B), but at a price of retaining more candidate types for downstream assessment. The results show that for prediction of most tumor types, the correct type ranks between 1-1.5 on average, and the average number of types within range is also between 1-1.5 (FIG. 8). Hence the within range approach is more economical than rank ≤2, as fewer than 2 types will typically be retained for further consideration, while delivering comparable performance (whereas rank ≤2, is primarily defined to retain 2 types). The ability of either of these extensions to enhance recovery of the correct type may be clinically valuable on a per-sample basis, especially when blood-based assessment precedes other diagnostic modalities.

### Example 5

### Performance of 8-Marker Tumor-Normal Panel

This example describes a set of loci/probes whose methylation not only distinguishes tumors from healthy blood but whose methylation in healthy tissues is similar to that in blood. Thus, when tumor methylation deviates from normal methylation is detected. The eight T-N call probes are identified to distinguish the tumors and normal samples, inclusive of peripheral blood and tissue samples (see above). When applied to the discovery dataset, the panel correctly identified 91.4% of tumor samples (FIG. 2A). In three tumor types, colorectal, stomach and uterine tumors, over 95% of samples were correctly identified. Fewer samples were correctly identified in pancreatic tumors, 74% (48/65). All samples from peripheral blood were identified correctly as non-tumor as were 98.8% of normal tissue samples. The false-positive rate for normal tissue samples from stomach, pancreas, lung, kidney, liver, and uterus was zero, whereas it rose in prostate tissue to 6.1% (FIG. 2B).

### Example 6

### Performance of 46-Marker Combination Panel

Next, the 8-marker T-N call panel, coupled to the 39-marker classification panel, was used to assess samples in concert. This yields a 46-marker panel for classification (where one marker was present in both panel sets). All the samples called as normal by the 8-marker panel were assigned a final classification as reference (i.e., non-tumor). This combination panel correctly detected and classified samples from the 13 TCGA solid tumor types from the discovery set at a rate of 68 to 93%, depending on tumor type, with a slightly worse performance than the 39-marker panel alone. As was true of the 39-marker panel, many misclassified samples were identified as a similar or related tumor type from the same or an adjacent organ. The 46-marker panel misclassified larger fractions of tumor samples as reference samples, due to the performance of the 8-marker panel. For example, the same 26% of pancreatic tumors were now classified as reference as were reported for the 8-marker panel alone vs. 8% of pancreatic tumors for the 39-marker panel. Also, normal tissue samples were incorrectly identified as tumor samples only 1.2% of the time, as with the 8-panel marker. The 46-marker panel correctly assigned all peripheral blood reference samples as normal.

### Example 7

### Performance of Panels when Applied to Validation Dataset

The 39- and 8-marker panels were applied to a more diverse set of eight independently generated cancer datasets, consisting of both array and whole genome bisulfite sequencing data. These datasets contained 908 tumor samples containing colon, pancreas, lung (unspecified subtype), breast, kidney clear cell, liver, and prostate tissues; 275 normal samples containing colon, pancreas, lung, breast and kidney tissues, and 32 normal blood plasma samples (Table 2).

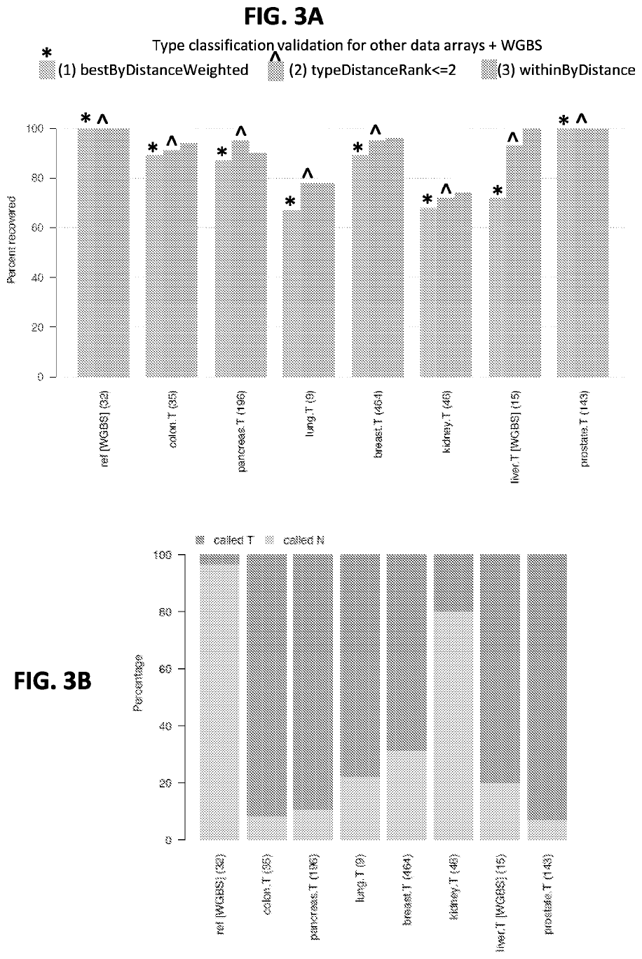

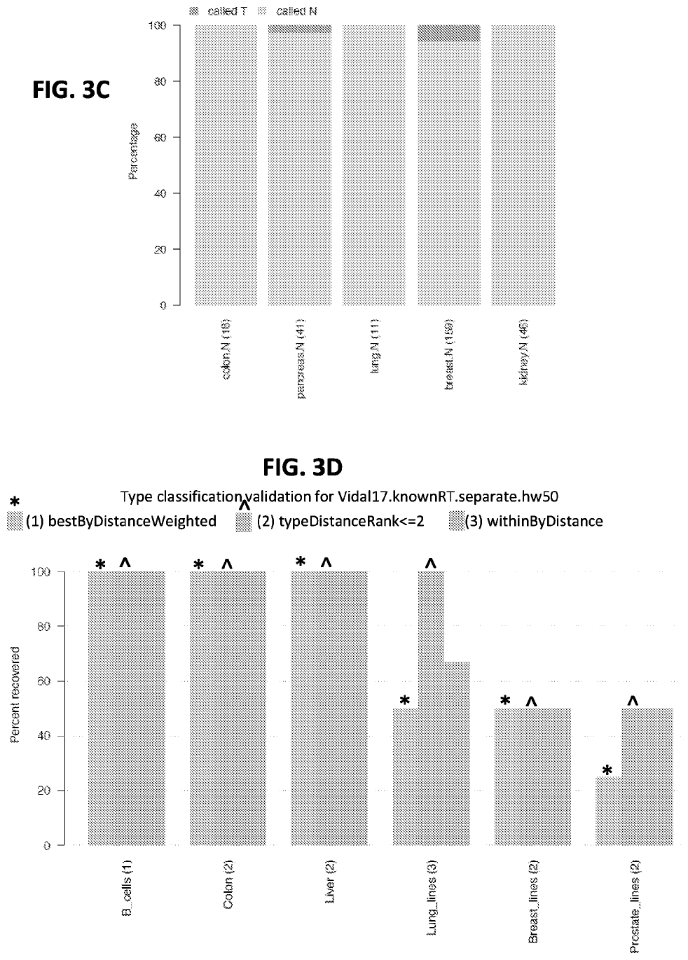

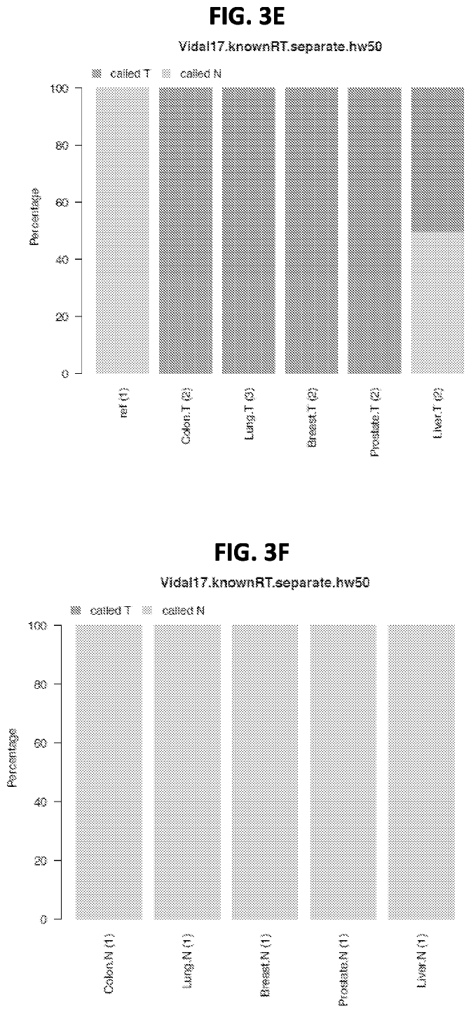

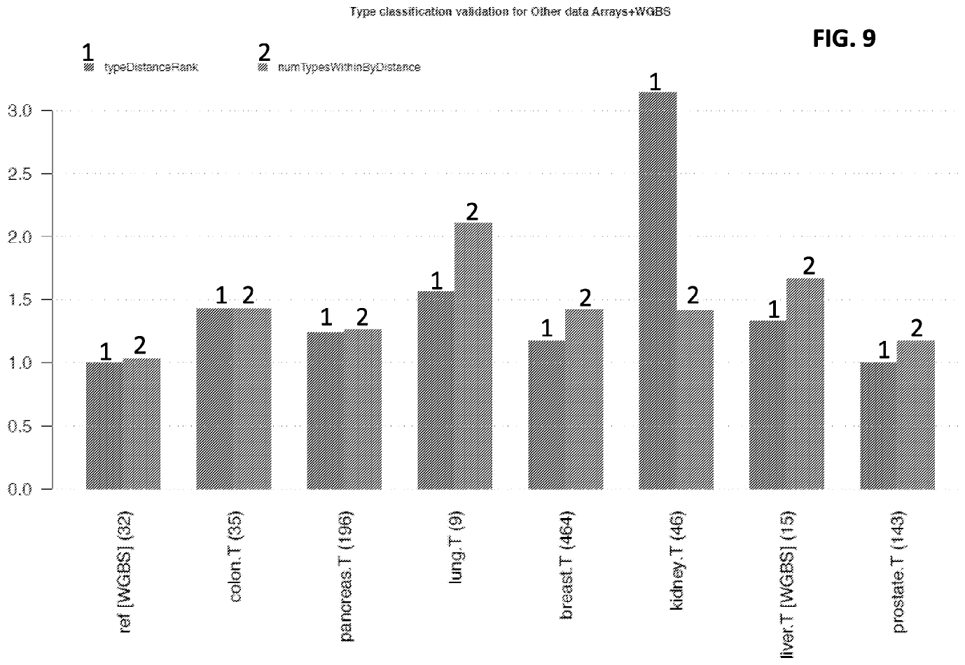

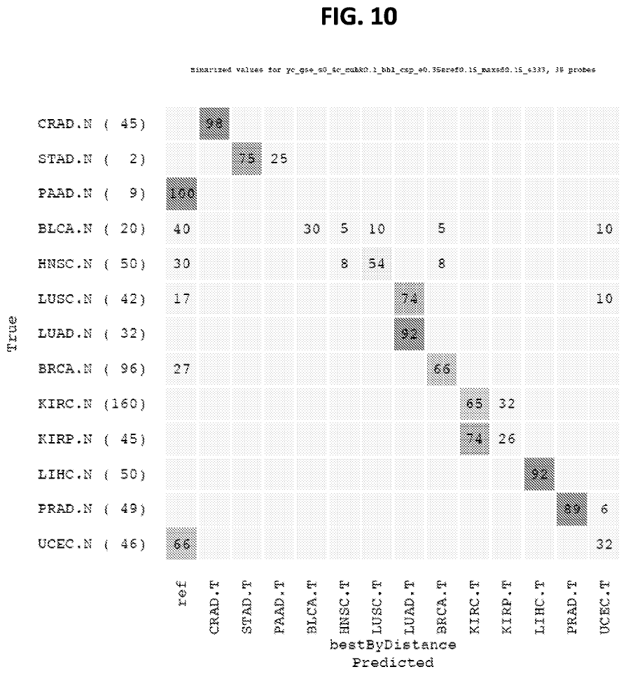

Overall the classification performance was comparable to the results obtained from the discovery dataset. For example, the 39-marker panel classified a median of 87% of tumor samples correctly (range 67-100%; FIG. 3A, orange). The colon, pancreas, breast, and prostate tumor samples were classified correctly 87-100% of the time. Lung, kidney, and liver classification performed worse with 67-72% correctly classified (assuming the unspecified lung samples to be adenocarcinoma), however encompassing additional likely predictions substantially improved correct type recovery (FIG. 3B, gray and green; FIG. 9). Cross-classification was seen in this dataset, for example, 22% of lung samples classified as lung squamous cell and 5% of kidney tumor samples classified as kidney renal cell. In normal blood plasma samples, all 32 samples were classified as reference using this panel. Likewise, the 8-marker panel correctly detected 69-93% of tumor samples, excluding kidney tumors which had a 20% detection (FIG. 3B). With kidney tumors excluded, the median correct classification rate was 85%. When considering the known sample identities, fewer than 10% of colon and prostate tumor samples were identified incorrectly as normal samples. Considering normal tissue samples, only 10/275 (3.6%) of were incorrectly identified as tumor samples, and these misclassifications were limited to pancreas (1/41) and breast (9/159) tissue samples. In normal blood plasma samples, only one out of 32 was called a tumor (FIG. 3B, 3C).

In order to further estimate the performance of the 8- and 39-marker panels on WGBS data, 17 additional samples were analyzed. These samples include one normal sample each of B cells, breast, colon, liver, lung and prostate (n=6), as well as two each of colorectal, liver, breast, and prostate cancers, and three lung cancer samples (lung adenocarcinoma, squamous cell and small cell lung cancer) (n=11) (Vidal et al., Oncogene, 36(40):5648-5657, 2017). DNA from normal tissues, colorectal and liver cancers was harvested from the tissue samples whereas the rest were collected from cell lines. The 39-mrker tumor type classification panel correctly classified all 6 normal samples (by tissue) and 8 tumor samples, missing on one breast and one prostate cancer cell line. The small cell lung cancer line (the lung cancer subtype not explicitly considered by us) had LUSC as its 2^{nd} ranked classification (FIG. 3D). The 8-marker tumor detection panel performed correctly on all samples except one liver carcinoma that went undetected (FIG. 3E, 3F).

### Example 8

### Performance Evaluation

The reasons for poor performance of some sample sets in the validation data were evaluated and it was concluded that data quality affected the results. For example, in the 39-marker panel, data for 9 of 39 markers were absent across all kidney samples (FIG. 3A). Further, the liver samples were analyzed using a different method, whole genome bisulfite sequencing, WGBS. Although the rate of correct classification for WGBS liver tumor samples appeared low (72%), when all predictions were done using the “within range” approach the correct classification was made in all cases (FIG. 3A).

The 8-marker panel performance was poor in kidney tumors, where data for 3 of 8 markers were absent (two of the missing markers were specifically discriminative for kidney cancers) (FIG. 3B). Data quality issues also affected samples in addition to kidney samples. For example, pancreas tumor samples lacked data at 2 of 39 tumor-type classification markers and 1 of 8 tumor detection markers, yielding 87% correct classification and 92% detection rates, respectively. Also, of the 9 control breast samples called as tumors, 8 were from adjacent tissues, and only 1 from a healthy donor. Nevertheless, there were comparable numbers of healthy (50) and adjacent normal controls (52) in the datasets containing these 9 false positive calls.

### Example 9

### Performance of Panels on Non-Cancer Conditions

In addition to tumor samples described above, tissue samples from several non-cancer conditions were assessed: type 2 diabetes, asthma, chronic kidney disease, non-alcoholic fatty liver disease and endometriosis, as well as peripheral blood from individuals with Crohn's disease or ulcerative colitis (Table 3). All but the liver disease samples were accompanied by normal control samples. None of 200 affected samples and 165 controls were identified as tumor samples using the 8-marker panel (Table 3), and all blood samples were classified as reference by type, using the 39-marker panel.

TABLE 3
Validation samples: non-cancer conditions and control samples

| | | Sample | Called as | Called as | |
|---|---|---|---|---|---|
| Data Source | Description | Count | tumor | normal | Format |
| GSE32148 | peripheral blood: | 17 | 0 | 17 | Infinium 450K array |
| | Crohns' disease | | | | |
| | peripheral blood: | 11 | 0 | 11 | Infinium 450K array |
| | ulcerative colitis | | | | |
| | peripheral blood: | 20 | 0 | 20 | Infinium 450K array |
| | normal controls | | | | |
| GSE85566 | airway epithelial | 74 | 0 | 74 | Infinium 450K array |
| | cells: asthma | | | | |
| | airway epithelial | 41 | 0 | 41 | Infinium 450K array |
| | cells: control | | | | |
| GSE50874 | chronic kidney | ~20^{a} | 0 | ~20^{a} | Infinium 450K array |
| | disease | | | | |
| | kidney control | ~65^{a} | 0 | ~65^{a} | Infinium 450K array |
| GSE49542 | non-alcoholic | 59 | 0 | 59 | Infinium 450K array |
| | fatty liver | | | | |
| | disease | | | | |
| GSE87621 | endometriosis | 4 | 0 | 4 | Infinium 450K array |
| | [cell culture] | | | | |
| | control [cell | 5 | 0 | 5 | Infinium 450K array |
| | culture] | | | | |
| PMID: | pancreatic islets: | 15 | 0 | 15 | Infinium 450K array |
| 24603685 | T2D | | | | |
| | pancreatic islets: | 34 | 0 | 34 | Infinium 450K array |
| | non-diabetic | | | | |

^{a}The source paper states that “External validation was performed on 87 microdissected human kidney tubule epithelial samples, 21 samples from patients with DKD and 66 controls (including hypertension (n = 22), diabetes mellitus (n = 22) or none (n = 22))”. The data are for 85 samples without identification.

### Example 10

### Performance of Panels Using Amplicon-Based Bisulfite Sequencing

To further assess the utility of the 8- and 39-merker panels, it was tested whether methylation levels at the identified markers were useful when measured via amplicon-based bisulfite sequencing. This targeted sequencing enables simultaneous interrogation of adjacent CpGs on the same strand of DNA, which is useful in a blood-based application to enhance signal detection. Using two runs of the Fluidigm platform (see Methods), 4 or 5 tumor and 2 or 3 normal samples were assessed for eight different types of solid tissue (colon, stomach, pancreas, lung, breast, kidney, liver and prostate), as well as 13 normal blood samples. This type of assay offers a nonmultiplexed, high-throughput, low-volume means of analyzing up to 48 markers from up to 48 samples in a single run.

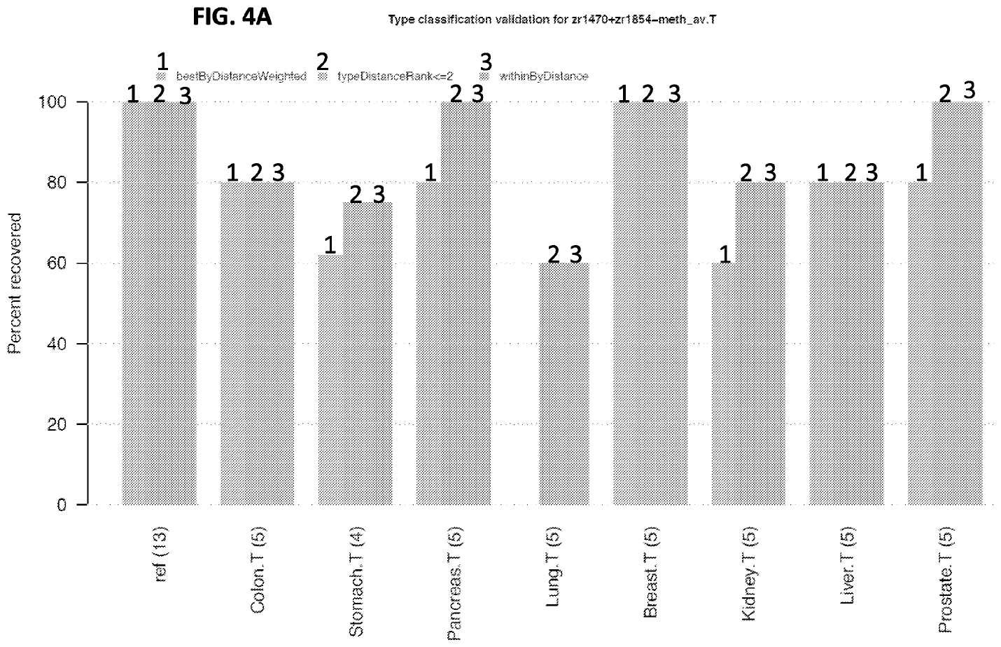

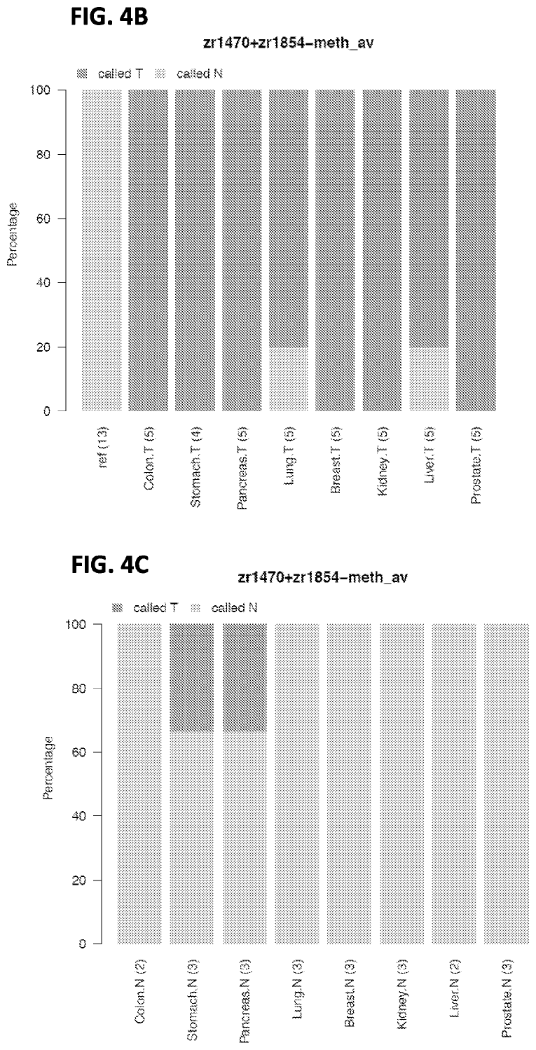

With the 39-marker panel, breast tumors and blood reference were 100% correctly classified by type, while colon, pancreas liver and prostate tumors classified 80% correct. Kidney and lung tumor samples had the worst performances, with all lung samples misclassified (4/5 as breast and 1 as reference) (FIG. 4A). This result appeared to be due to technical issues, mainly poor amplification performance of some amplicons. Nevertheless, retaining additional candidate classifications, either those with rank 2, or alternatively those “within range”, improved recovery of the correct type for stomach, pancreas, kidney, prostate and lung, with pancreas and prostate reaching 100% and lung reaching 60% (FIG. 4A). It was observed that the average rank of correct type, as well as the average number of types in range remained similar to the values reported for the array data, except for lung, where they rose to 2.6 and 2.2, respectively.

With the 8-marker panel, 6 out of 8 tumor types were 100% correctly identified as tumors, while lung and liver had one false negative each (FIG. 4B). For normal samples, colon, lung, breast kidney, liver and prostate were 100% correct, whereas stomach and pancreas each had one false positive sample (FIG. 4C). All blood samples were correctly identified as reference (FIG. 4B). Overall, 54/58 (93%) samples were correctly identified (91% when excluding 13 blood samples). For this assays presented in FIGS. 4B and 4C, the primers used to amplify the 8-marker panel from genomic DNA were the forward and reverse primers set forth as SEQ ID NOs: 1-16.

### Example 11

### Performance of Panels on Cell Free DNA from Patient Plasma

Given the successful classification of the probe panels on tumor and normal tissues and normal peripheral blood samples, plasma samples from patients with or without cancer were examined. The data assessed came from publically available whole genome bisulfite sequencing data from plasma samples of patients with hepatocellular (liver) cancer (Chan et al (PNAS, 110(47):18761-18768, 2013).

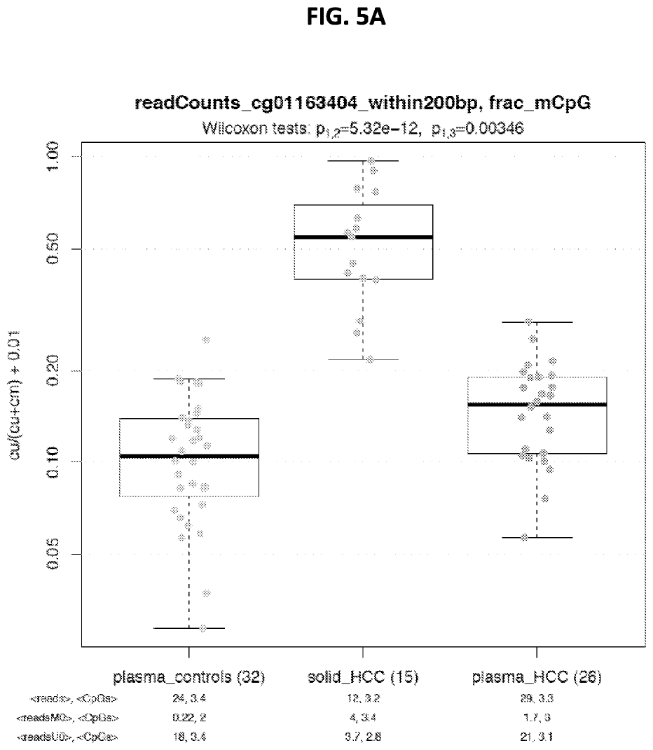

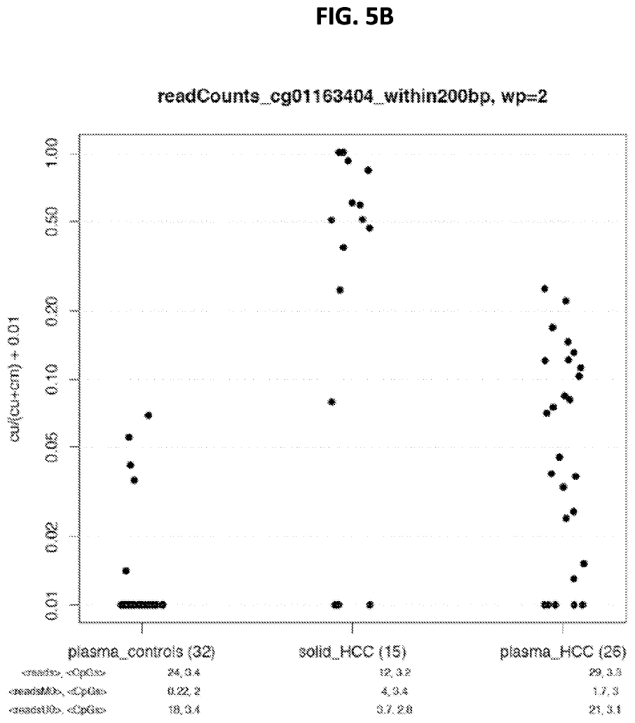

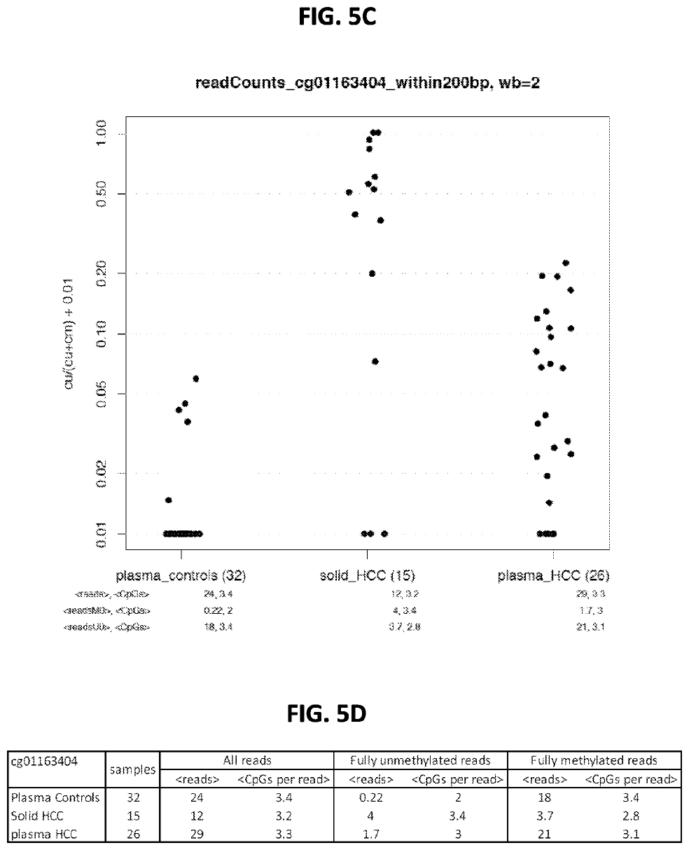

For the 8-probe panel, the methylation in individual sequence reads for 200 bp intervals around the original CpG probe site were examined. It was found that the average methylation differed significantly between plasma controls and plasma hepatocellular cases (p<0.05 after Holm's correction) in six out of the eight loci. An example for one of the loci is shown in FIG. 5. A sample was called as a tumor if at least one of the probes had a signal above all normal controls and detected ˜16/26 (61.5%) of plasma hepatocellular cases (varying from 15-17, depending on the exact method of signal detection—see Methods). When plasma controls were swapped for HCC plasma samples using the same algorithm to ask if any controls were called positive, generally no controls were called positive (when using one of the considered signal detection methods, defined by no read weighting (r=0; see Methods), one of the 32 controls was called positive), further indicating detectable HCC signal was specific for the tumor plasma.

Using the 39-probe panel for tumor-type classification, however, classifies overwhelming majority (19-25) of the 26 plasma HCCs as reference. Those that are classified differently, tend to classify to types closest to the reference class (mainly BRCA.T). This is not surprising as for the correct classification, many probes must show concordant signal which is unlikely with shallow coverage. Absence of signal in multiple loci makes the sample look like reference. Nevertheless, there is (statistically significantly) more signal in the probes that should have it for LIHC.T (HCC) than in those that should not (p<0.05 after Holm's correction, Spearman correlation test or Wilcoxon rank sum test; see Methods), indicating that some signal is present.

### Example 12

### Multiplex Amplification of 8-Marker Panel

This example describes a multiplex assay for amplification of the 8-marker panel,

As discussed above, the methylation status of CpG sites located at chr6:88876741, chr6:150286508, chr7:19157193, chr10:14816201, chr12:129822259, chr14:89628169, chr17:40333009, and chr17:46655394 can be used to assess cancer status in a subject.

To implement detection of the methylation status of these sites, primers were designed for multiplex amplification of genomic regions including each of chr6:88876741, chr6:150286508, chr7:19157193, chr10:14816201, chr12:129822259, chr14:89628169, chr17:40333009, and chr17:46655394. The primers design was optimized for amplification with an annealing temperature of 56° C. The primers are shown in the following table.

| Target | Forward primer | Reverse primer |
|---|---|---|
| chr6:88876741 | GGGTYGGGTTG | CCTACRAAACC |
| | AGTTTTGGGAT | CAACTATTTAC |
| | (SEQ ID | AT |
| | NO: 1) | (SEQ ID |
| | | NO: 2) |
| chr6:150286508 | YGTGTTTGGTT | CCTATCCTAAC |
| | GAAGGTATTTA | CCCAACTAAAA |
| | G | TT |
| | (SEQ ID | (SEQ ID |
| | NO: 3) | NO: 4) |
| chr7:19157193 | ATGGTTTYGAG | TCAAACCAATA |
| | GTTTAAAAAGA | ACACTACTACC |
| | AAG | C |
| | (SEQ ID | SEQ ID |
| | NO: 5) | NO: 6) |
| chr10:14816201 | TTYGGGTTTTT | ATCCACATCTT |
| | GGATGATGGGG | TTAAAAACACT |
| | (SEQ ID | CTAAAA |
| | NO: 7) | ATCTACA |
| | | (SEQ ID |
| | | NO: 8) |
| chr12:129822259 | AGAGTTTGGTG | ACTATCCTAAT |
| | ATTTGTTAGTT | TCTTAACTCCT |
| | ATATAGT | CCCCT |
| | TGG | (SEQ ID |
| | (SEQ ID | NO: 10) |
| | NO: 9) | |
| chr14:89628169 | GATGGTGTTAG | TACACAAAACC |
| | GAAAGTTATTG | AATCTTCAAAC |
| | GAATTGT | TTATAA |
| | T | CTTTTAA |
| | (SEQ ID | (SEQ ID |
| | NO: 11) | NO: 12) |
| chr17:40333009 | GTTTGTYGGGA | AAATAAAACRA |
| | TTTTGGGTTTT | ACTAAAATACA |
| | (SEQ ID | AAAAAT |
| | NO: 13) | TCTAAA |
| | | (SEQ ID |
| | | NO: 14) |
| chr17:46655394 | AGTTYGAGGGG | AAACCAACAAC |
| | AAGAATTTGGT | CCCTTCATAAC |
| | (SEQ ID | (SEQ ID |
| | NO: 15) | NO: 16) |

The amplicons generated using these primers are as follows (with and without primer sequence):

| | Target amplified sequence |
|---|---|
| Target | (with primer) |
| chr6:88876741 | GGGCCGGGCTGAGCTTTGGGACCGC |
| | GCTGCGCAGCCCCCGAGCCGTTCGC |
| | TACCTGGCTGCGGTCGCGCGGCCAC |
| | CTGTCCTCCGCCCTCGGGGCGCCGC |
| | CCAGCTTCGCGCCAGGCGCCTTCT |
| | CCAGCGCCCGCCGCCCTTTCCCGG |
| | CGAGACCACTCGggCGCGcccCGc |
| | cCGcCGgTCCCCGATGCAAACAGT |
| | TGGGCTCCGTAGG |
| | (SEQ ID NO: 17) |
| chr6:150286508 | CGTGCCTGGCTGAAGGCACTCAGTT |
| | CCCCTCCGGGGCTCCTTTCCGCCGA |
| | GTCCGCTTCCTGCAGCTGCTGCTAG |
| | CACCGCAGTCCAGGGGGAGTGTCAA |
| | AGAAGGCTGAAAAGGAATTGCAGGA |
| | GGGTGGAGGGACCAAAAGGCTACAG |
| | AGGGCAAGGTAGGGCGGGGATCCCT |
| | GGTGCAGACCCGCAGCCCCACTGGC |
| | CCTAGGGAAGGAGAAACCAGATTCC |
| | CGAACCCTAGCTGGGGTCAGGACAG |
| | G |
| | (SEQ ID NO: 18) |
| chr7:19157193 | ATGGCCCCGAGGTCCAAAAAGAAAG |
| | CGCCCAACGGCTGGACGCACACCCC |
| | GCCAGGCCTCCTGGAAACGGTGCCG |
| | GTGCTGCAGAGCCCGCGAGGTGTCT |
| | GGGAGTTGGGCGAGAGCTGCAGACT |
| | TGGAGGCTCTTATACCTCCGTGCAG |
| | GCGGAAAGTTTGGGGGCAGCAGTGT |
| | CATTGGCCTGA |
| | (SEQ ID NO: 19) |
| chr10:14816201 | CCCGGGTCCCTGGATGATGGGGCGG |
| | ACTGTGAAGCAGTGGTGTTTCACGC |
| | TTCCATCCCCAGACCATCAATTATT |
| | GACACGCCCAAGGTGAGTGAGTGTT |
| | CGCTCTGCGATTATAGACGGGATGG |
| | AGCTGGAGGAGCGTTGGGATCATGT |
| | GGCGAATGTTTCAGCAAACAAACTC |
| | ATTTAACCTTACTGAATAAGGCATT |
| | GCGGGCGTTCTCACTAGTGCGAAGA |
| | AGTGTGTTAAAGCCGTGTAGATTCT |
| | CAGAGTGTTCTCAAAAGATGTGGAT |
| | (SEQ ID NO: 20) |
| chr12:129822259 | AGAGCCTGGTGACCTGCCAGCCACA |
| | CAGCTGGTCACGTGGCAGGTCGAGT |
| | ACCCCGGAGAGATCACGTCTGACTT |
| | GGGAGTGTCCAAGATCTATGTGAGC |
| | CCAAAGGACTTGATTGGAGTTGTGC |
| | CGCTGGCTATGGTAAGCAAGCCCCG |
| | CCCTGGGCTGTTAGAACTGAACTCG |
| | GGGGAGGGGAAGGCGCCGGCGCCGC |
| | ACTGAGTCCCAGGCTGGGTGGGGAA |
| | AAGGGGAGGAGCCAAGAATCAGGAC |
| | AGT |
| | (SEQ ID NO: 21) |
| chr14:89628169 | GATGGTGCCAGGAAAGCCACTGGAA |
| | TTGTCACACGGCGAGCACAGAGGGC |
| | CGGCCACCAGTCCTCGATGCTTCTG |
| | AACCCTGAAGCCCGATGACATCTTA |
| | CGAGGTGGACGTTGGACTGTTCATG |
| | CGCATCGGGTGTCAGTGACTCATGG |
| | AGAAGAAATGGGGTAAATTTTTAGT |
| | GATGTTGCTAATCATTGAATTCTGT |
| | TCTCTATTAAATTAAGAAAATGTTC |
| | CAAAAGCCATAAGCCTGAAGATTGG |
| | CCCTGTGca |
| | (SEQ ID NO: 22) |
| chr17:40333009 | GCCTGCCGGGACCCTGGgccccCGc |
| | CGcctcCGccaccaccccCGcCGcc |
| | ccCGccacCGccCGGTCTGTCCCCT |
| | CGGGCTCCTGCGCCGCCACCCGCCG |
| | GGGCCCTCCTCCCGGAGCCCGGCCA |
| | GCGCTGCGAGGCGGTCAGCAGCAGC |
| | CCCCCGCCGCCTCCCTGCGCCCAGA |
| | ACCCCCTGCACCCCAGCCCGTCCCA |
| | CTC |
| | (SEQ ID NO: 23) |
| chr17:46655394 | AGCCCGAGGGGAAGAACCTGGCCCG |
| | TGGGGAGGTGGGGGGGACCGAAACG |
| | GCGCTGAGCCGAGCCGAGAGCTACG |
| | GGGTTCGGAGCAGAGGCAGCGGCAG |
| | CGGCAGCGGCAGTAAGAGGGAGGGG |
| | AGGAGGCAGGAGGGCGCATggggCG |
| | cccCGgcccctcCGacagCGCGccc |
| | cctcCGgccCGgcCGCGcTGAAAGC |
| | TCCCCAGCGCCGCGCCTTGAACCCA |
| | CGCCCCGGGGCCATGCCGGTCATGA |
| | AGGGGTTGCTGGCCC |
| | (SEQ ID NO: 24) |
| | Target amplified sequence |
| Target | (without primer) |
| chr6:88876741 | CGCGCTGCGCAGCCCCCGAGCCGTT |
| | CGCTACCTGGCTGCGGTCGCGCGGC |
| | CACCTGTCCTCCGCCCTCGGGGCGC |
| | CGCCCAGCTTCGCGCCAGGCGCCTT |
| | CTCCAGCGCCCGCCGCCCTTTCCCG |
| | GCGAGACCACTCGggCGCGcccCGc |
| | cCGcCGgTCCCCG |
| | (SEQ ID NO: 25) |
| chr6:150286508 | TTCCCCTCCGGGGCTCCTTTCCGCC |
| | GAGTCCGCTTCCTGCAGCTGCTGCT |
| | AGCACCGCAGTCCAGGGGGAGTGTC |
| | AAAGAAGGCTGAAAAGGAATTGCAG |
| | GAGGGTGGAGGGACCAAAAGGCTAC |
| | AGAGGGCAAGGTAGGGCGGGGATCC |
| | CTGGTGCAGACCCGCAGCCCCACTG |
| | GCCCTAGGGAAGGAGAAACCAGATT |
| | CCCG |
| | (SEQ ID NO: 26) |
| chr7:19157193 | CGCCCAACGGCTGGACGCACACCCC |
| | GCCAGGCCTCCTGGAAACGGTGCCG |
| | GTGCTGCAGAGCCCGCGAGGTGTCT |
| | GGGAGTTGGGCGAGAGCTGCAGACT |
| | TGGAGGCTCTTATACCTCCGTGCAG |
| | GCGGAAAGTTTGG |
| | (SEQ ID NO: 27) |
| chr10:14816201 | CGGACTGTGAAGCAGTGGTGTTTCA |
| | CGCTTCCATCCCCAGACCATCAATT |
| | ATTGACACGCCCAAGGTGAGTGAGT |
| | GTTCGCTCTGCGATTATAGACGGGA |
| | TGGAGCTGGAGGAGCGTTGGGATCA |
| | TGTGGCGAATGTTTCAGCAAACAAA |
| | CTCATTTAACCTTACTGAATAAGGC |
| | ATTGCGGGCGTTCTCACTAGTGCGA |
| | AGAAGTGTGTTAAAGCCG |
| | (SEQ ID NO: 28) |
| chr12:129822259 | TCACGTGGCAGGTCGAGTACCCCGG |
| | AGAGATCACGTCTGACTTGGGAGTG |
| | TCCAAGATCTATGTGAGCCCAAAGG |
| | ACTTGATTGGAGTTGTGCCGCTGGC |
| | TATGGTAAGCAAGCCCCGCCCTGGG |
| | CTGTTAGAACTGAACTCGGGGGAGG |
| | GGAAGGCGCCGGCGCCGCACTGAGT |
| | CCCAGGCTGGGTGGGGAAA |
| | (SEQ ID NO: 29) |
| chr14:89628169 | ACACGGCGAGCACAGAGGGCCGGCC |
| | ACCAGTCCTCGATGCTTCTGAACCC |
| | TGAAGCCCGATGACATCTTACGAGG |
| | TGGACGTTGGACTGTTCATGCGCAT |
| | CGGGTGTCAGTGACTCATGGAGAAG |
| | AAATGGGGTAAATTTTTAGTGATGT |
| | TGCTAATCATTGAATTCTGTTCTCT |
| | ATTAAATTAAGAAAATGTT |
| | (SEQ ID NO: 30) |
| chr17:40333009 | CGcCGcctcCGccaccaccccCGcC |
| | GccccCGccacCGccCGGTCTGTCC |
| | CCTCGGGCTCCTGCGCCGCCACCCG |
| | CCGGGGCCCTCCTCCCGGAGCCCGG |
| | CCAGCGCTGCGAGGCGGTCAGCAGC |
| | AGCCCCCCGCCGCCTCCCTGCG |
| | (SEQ ID NO: 31) |
| chr17:46655394 | CCGTGGGGAGGTGGGGGGGACCGAA |
| | ACGGCGCTGAGCCGAGCCGAGAGCT |
| | ACGGGGTTCGGAGCAGAGGCAGCGG |
| | CAGCGGCAGCGGCAGTAAGAGGGAG |
| | GGGAGGAGGCAGGAGGGCGCATggg |
| | gCGcccCGgcccctcCGacagCGCG |
| | ccccctcCGgccCGgcCGCGcTGAA |
| | AGCTCCCCAGCGCCGCGCCTTGAAC |
| | CCACGCCCCGGGGCCATGCCG |
| | (SEQ ID NO: 32) |

The multiplex PCR assay is intended for use with samples containing cell-free DNA, such as blood or plasma samples, although other types samples can also be used. Prior to amplification, the sample is treated with bisulfite to convert unmethylated cytosines to uracil, which are amplified as thymine.

The presence of the thymine (or adenine-thymine base pair) in place of cytosine is detected to indicate that the cytosine was not methylated in the sample. Non-limiting detection methods include amplicon sequencing (e.g., as described above), fluorescence, agarose gel separation, high resolution melting (such as the DREAMing method described in Pisanic et al., “Dreaming a simple and ultrasensitive method for assessing intratumor epigenetic heterogeneity directly from liquid biopsies,” Nucleic Acids Research, 43(22):e154, 2015).

The results of the detection procedure are used to assign a status of altered or normal methylation to each of the genomic segments. Detection of a significant change (increase or decrease) in methylation of the segment compared to normal control is used assign the status of altered or not.

Liquid biopsy samples such as blood and plasma from a patient with cancer contain intrinsically low numbers of circulating tumor DNA. High-resolution epigenetic analysis, for example, using the DREAMing method, is used to detect single copy variation in methylation status from liquid biopsy samples, which can be used to assign the normal or altered methylation status for each of the genomic segments.

As discussed above, in normal tissue, CpG sites located at genomic segments containing chr10:14816201, chr12:129822259, and chr14:89628169 are methylated and CpG sites located at genomic segments containing chr17:40333009, chr17:46655394, chr6:88876741, chr6:150286508, and chr7:19157193 are not methylated. Deviation from this normal status is used for assignment of altered methylation status.

The biological sample is identified as from a subject with cancer if at least one of the genomic segments is assigned an altered methylation status, and the biological sample is identified as from a subject without cancer if none of the genomic segments are assigned an altered methylation status.

We claim all subject matter that comes within the scope and spirit of the claims below. Alternatives specifically addressed in these sections are merely exemplary and do not constitute all possible alternatives to the embodiments described herein.

1. A method, comprising:
obtaining a plurality of sequence reads of a methylation sequencing assay covering genomic segments of a biological sample from a human subject, wherein the genomic segments contain the following genomic positions: chr6:88876741, chr6:150286508, chr7:19157193; chr10:14816201, chr12:129822259, chr14:89628169, chr17:40333009, and chr17:46655394 according to a GRCh37/hg19 reference human genome;
assigning a methylation status of altered or normal to each of the genomic segments by comparing methylation of CpG sites of the sequence reads covering the respective genomic segments to a normal control; and
identifying the biological sample as from a subject with cancer if at least one of the genomic segments is assigned an altered methylation status, or
identifying the biological sample as from a subject without cancer if none of the genomic segments are assigned an altered methylation status.

2. The method of claim 1, wherein:
assigning a methylation status to the genomic segments containing chr17:40333009, chr17:46655394, chr6:88876741, chr6:150286508, and chr7:19157193 comprises calculating a ratio X₁ according to:
X₁=F₂/(F₁+F₂)
wherein F₁ and F₂ are frequencies of sequence reads in the plurality corresponding to a genomic segment where less than 40% or at least 60% of the CpG sites are methylated, respectively, and wherein a genomic segment is assigned an altered methylation status if there is an increase in the ratio X₁ compared to the normal control and a genomic segment is assigned a normal methylation status if there is not an increase in the ratio X₁ compared to the normal control; and
assigning a methylation status to the genomic segments containing chr10:14816201, chr12:129822259, and chr14:89628169 comprises calculating a ratio X₂ according to:
X₂=F₁/(F₁+F₂)
wherein F₁ and F₂ are as defined above, and wherein a genomic segment is assigned an altered methylation status if there is an increase in the ratio X₂ compared to the normal control and a genomic segment is assigned a normal methylation status if there is not an increase in the ratio X₂ compared to the normal control.

3. The method of claim 1, wherein:
assigning a methylation status to the genomic segments containing chr17:40333009, chr17:46655394, chr6:88876741, chr6:150286508, and chr7:19157193 comprises calculating a ratio X₃ according to:
X₃=F₄/(F₃+F₄)
wherein F₃ and F₄ are frequencies of sequence reads in the plurality corresponding to a genomic segment where less than 20% or at least 80% of the CpG sites are methylated, respectively, and wherein a genomic segment is assigned an altered methylation status if there is an increase in the ratio X₃ compared to the normal control and a genomic segment is assigned a normal methylation status if there is not an increase in the ratio X₃ compared to the normal control; and
assigning a methylation status to the genomic segments containing chr10:14816201, chr12:129822259, and chr14:89628169 comprises calculating a ratio X₄ according to:
X₄=F₃/(F₃+F₄)
wherein F₃ and F₄ are as defined above, and wherein a genomic segment is assigned an altered methylation status if there is an increase in the ratio X₄ compared to the normal control and a genomic segment is assigned a normal methylation status if there is not an increase in the ratio X₄ compared to the normal control.

4. The method of claim 1, wherein:
assigning a methylation status to the genomic segments containing chr17:40333009, chr17:46655394, chr6:88876741, chr6:150286508, and chr7:19157193 comprises calculating a ratio X₅ according to:
X₅=F₆/(F₅+F₆)
wherein F₅ and F₆ are frequencies of sequence reads in the plurality corresponding to a genomic segment where none or all of the CpG sites are methylated, respectively, and wherein a genomic segment is assigned an altered methylation status if there is an increase in the ratio X₅ compared to the normal control and a genomic segment is assigned a normal methylation status if there is not an increase in the ratio X₅ compared to the normal control; and
assigning a methylation status to the genomic segments containing chr10:14816201, chr12:129822259, and chr14:89628169 comprises calculating a ratio X₆ according to:
X₆=F₅/(F₅+F₆)
wherein F₅ and F₆ are as defined above, and wherein a genomic segment is assigned an altered methylation status if there is an increase in the ratio X₆ compared to the normal control and a genomic segment is assigned a normal methylation status if there is not an increase in the ratio X₆ compared to the normal control.

5. The method of claim 2, wherein the increase in the ratios X₁ and X₂, X₃ and X₄, and/or X₅ and X₆ compared to the normal control is an increase of at least 50% and/or an increase of at least two standard deviations.

6. (canceled)

7. The method of claim 1, wherein the genomic segments are plus or minus up to 300 bases of the genomic positions and/or plus or minus 50 to 300 bases of the genomic positions.

8. (canceled)

9. The method of claim 1, comprising identifying the biological sample as from a subject with cancer if at least two of the genomic segments is assigned an altered methylation status.

10. The method of claim 1, wherein the methylation sequencing assay is a bisulfite sequencing assay.

11. The method of claim 1, wherein the biological sample is a whole blood, serum, plasma, buccal epithelium, saliva, urine, stools, ascites, cervical pap smears, or bronchial aspirates sample.

12. (canceled)

13. The method of claim 1, wherein the biological sample contains cell-free DNA comprising the genomic segments.

14. The method of claim 1, wherein the genomic segments are PCR amplified prior to sequencing.

15. The method of claim 1, wherein the cancer is selected from colon cancer, rectal cancer, stomach cancer, pancreatic cancer, bladder cancer, head-neck cancer, lung cancer, breast cancer, kidney cancer, cervical cancer, liver cancer, uterine cancer, ovarian cancer, and prostate cancer.

16. The method of claim 1, further comprising obtaining the biological sample from the subject.

17. The method of claim 1, further comprising administering a therapeutically effective amount of an anti-cancer agent to the subject if the biological sample is identified as a sample from a subject with cancer.

18. The method of claim 1, implemented at least in part using a computer.

19. A computing system, comprising:
one or more processors;
memory; and
a classification tool configured to:
receive a plurality of sequence reads of a methylation sequencing assay covering genomic segments of a biological sample from a human subject, wherein the genomic segments contain the following genomic positions: chr6:88876741, chr6:150286508, chr7:19157193; chr10:14816201, chr12:129822259, chr14:89628169, chr17:40333009, and chr17:46655394 according to a GRCh37/hg19 reference human genome;
assign a methylation status of altered or normal to each of the genomic segments by comparing methylation of CpG sites of the sequence reads covering the respective genomic segments to a normal control; and
classify the biological sample as from a subject with cancer if at least one of the genomic segments is assigned an altered methylation status, or
classify the biological sample as from a subject without cancer if none of the genomic segments are assigned an altered methylation status.

20. A method, comprising:
providing a biological sample containing cell-free DNA from a human subject
treating the sample with bisulfite;
amplifying genomic segments from the bisulfite-treated sample, wherein the genomic segments contain the following genomic positions: chr6:88876741, chr6:150286508, chr7:19157193; chr10:14816201, chr12:129822259, chr14:89628169, chr17:40333009, and chr17:46655394 according to a GRCh37/hg19 reference human genome;
detecting methylation of the cell-free DNA corresponding to the genomic segments;
assigning a methylation status of altered or normal to the genomic segments; and
identifying the biological sample as from a subject with cancer if at least one of the genomic segments is assigned an altered methylation status, or
identifying the biological sample as from a subject without cancer if none of the genomic segments are assigned an altered methylation status.

21. The method of claim 20, wherein the genomic segments are plus or minus up to 300 bases of the genomic positions and/or plus or minus 50 to 300 bases of the genomic positions.

22. (canceled)

23. The method of claim 20, wherein the genomic segments containing chr6:88876741, chr6:150286508, chr7:19157193; chr10:14816201, chr12:129822259, chr14:89628169, chr17:40333009, and chr17:46655394 correspond to genomic sequence comprising or consisting of SEQ ID NOs: 25-32, respectively.

24. The method of claim 20, wherein amplifying the genomic segments comprises PCR amplification.

25. The method of claim 24, wherein the amplification is a single multiplex PCR amplification including amplification of each of the genomic segments.

26. The method of claim 20, wherein detecting methylation of the cell-free DNA corresponding the amplified genomic segments comprises sequencing the amplified genomic segments and/or a high-resolution PCR melt assay.

27. (canceled)

28. The method of claim 20, wherein:
amplifying the chr6:88876741 genomic segment comprises forward and reverse primers comprising or consisting of SEQ ID NOs: 1 and 2, respectively;
amplifying the chr6:150286508 genomic segment comprises forward and reverse primers comprising or consisting of SEQ ID NOs: 3 and 4, respectively;
amplifying the chr7:19157193 genomic segment comprises forward and reverse primers comprising or consisting of SEQ ID NOs: 5 and 6, respectively;
amplifying the chr10:14816201 genomic segment comprises forward and reverse primers comprising or consisting of SEQ ID NOs: 7 and 8, respectively;
amplifying the chr12:129822259 genomic segment comprises forward and reverse primers comprising or consisting of SEQ ID NOs: 9 and 10, respectively;
amplifying the chr14:89628169 genomic segment comprises forward and reverse primers comprising or consisting of SEQ ID NOs: 11 and 12, respectively;
amplifying the chr17:40333009 genomic segment comprises forward and reverse primers comprising or consisting of SEQ ID NOs: 13 and 14, respectively; and/or
amplifying the chr17:46655394 genomic segment comprises forward and reverse primers comprising or consisting of SEQ ID NOs: 15 and 16, respectively.

29. The method of claim 20, comprising identifying the biological sample as from a subject with cancer if at least two of the genomic segments is assigned an altered methylation status.

30. The method of claim 20, wherein the biological sample is a whole blood, serum, plasma, buccal epithelium, saliva, urine, stools, ascites, cervical pap smears, or bronchial aspirates sample.

31. The method of claim 30, wherein the biological sample is a blood or plasma sample.

32. The method of claim 20, wherein the cancer is selected from colon cancer, rectal cancer, stomach cancer, pancreatic cancer, bladder cancer, head-neck cancer, lung cancer, breast cancer, kidney cancer, cervical cancer, liver cancer, uterine cancer, ovarian cancer, and prostate cancer.

33. The method of claim 20, further comprising obtaining the biological sample from the subject.

34. The method of claim 20, further comprising administering a therapeutically effective amount of an anti-cancer agent to the subject if the biological sample is identified as a sample from a subject with cancer.

35. A kit comprising one or more primers comprising the amino acid sequence of any of SEQ ID NOs: 1-16, wherein the primers are up to 75 nucleotides in length.

36.-37. (canceled)


SEQUENCE LISTING (32 sequences)

SEQ ID NO: 1 - DNA, 22 bp, Artificial Sequence
  Primer
gggtygggttgagttttgggat

SEQ ID NO: 2 - DNA, 24 bp, Artificial Sequence
  Primer
cctacraaacccaactatttacat

SEQ ID NO: 3 - DNA, 23 bp, Artificial Sequence
  Primer
ygtgtttggttgaaggtatttag

SEQ ID NO: 4 - DNA, 24 bp, Artificial Sequence
  Primer
cctatcctaaccccaactaaaatt

SEQ ID NO: 5 - DNA, 25 bp, Artificial Sequence
  Primer
atggtttygaggtttaaaaagaaag

SEQ ID NO: 6 - DNA, 23 bp, Artificial Sequence
  Primer
tcaaaccaataacactactaccc

SEQ ID NO: 7 - DNA, 22 bp, Artificial Sequence
  Primer
ttygggtttttggatgatgggg

SEQ ID NO: 8 - DNA, 35 bp, Artificial Sequence
  Primer
atccacatcttttaaaaacactctaaaaatctaca

SEQ ID NO: 9 - DNA, 32 bp, Artificial Sequence
  Primer
agagtttggtgatttgttagttatatagttgg

SEQ ID NO: 10 - DNA, 27 bp, Artificial Sequence
  Primer
actatcctaattcttaactcctcccct

SEQ ID NO: 11 - DNA, 30 bp, Artificial Sequence
  Primer
gatggtgttaggaaagttattggaattgtt

SEQ ID NO: 12 - DNA, 35 bp, Artificial Sequence
  Primer
tacacaaaaccaatcttcaaacttataacttttaa

SEQ ID NO: 13 - DNA, 22 bp, Artificial Sequence
  Primer
gtttgtygggattttgggtttt

SEQ ID NO: 14 - DNA, 34 bp, Artificial Sequence
  Primer
aaataaaacraactaaaatacaaaaaattctaaa

SEQ ID NO: 15 - DNA, 22 bp, Artificial Sequence
  Primer
agttygaggggaagaatttggt

SEQ ID NO: 16 - DNA, 22 bp, Artificial Sequence
  Primer
aaaccaacaaccccttcataac

SEQ ID NO: 17 - DNA, 209 bp, Artificial Sequence
  amplicon with primer sequence
gggccgggctgagctttgggaccgcgctgcgcagcccccgagccgttcgctacctggctgcggtcgcgcggccacctgtcctccgccctcggggcgccgcccagcttcgcgccaggcgccttctccagcgcccgccgccctttcccggcgagaccactcgggcgcgccccgcccgccggtccccgatgcaaacagttgggctccgtagg

SEQ ID NO: 18 - DNA, 251 bp, Artificial Sequence
  amplicon with primer sequence
cgtgcctggctgaaggcactcagttcccctccggggctcctttccgccgagtccgcttcctgcagctgctgctagcaccgcagtccagggggagtgtcaaagaaggctgaaaaggaattgcaggagggtggagggaccaaaaggctacagagggcaaggtagggcggggatccctggtgcagacccgcagccccactggccctagggaaggagaaaccagattcccgaaccctagctggggtcaggacagg

SEQ ID NO: 19 - DNA, 186 bp, Artificial Sequence
  amplicon with primer sequence
atggccccgaggtccaaaaagaaagcgcccaacggctggacgcacaccccgccaggcctcctggaaacggtgccggtgctgcagagcccgcgaggtgtctgggagttgggcgagagctgcagacttggaggctcttatacctccgtgcaggcggaaagtttgggggcagcagtgtcattggcctga

SEQ ID NO: 20 - DNA, 275 bp, Artificial Sequence
  amplicon with primer sequence
cccgggtccctggatgatggggcggactgtgaagcagtggtgtttcacgcttccatccccagaccatcaattattgacacgcccaaggtgagtgagtgttcgctctgcgattatagacgggatggagctggaggagcgttgggatcatgtggcgaatgtttcagcaaacaaactcatttaaccttactgaataaggcattgcgggcgttctcactagtgcgaagaagtgtgttaaagccgtgtagattctcagagtgttctcaaaagatgtggat

SEQ ID NO: 21 - DNA, 253 bp, Artificial Sequence
  amplicon with primer sequence
agagcctggtgacctgccagccacacagctggtcacgtggcaggtcgagtaccccggagagatcacgtctgacttgggagtgtccaagatctatgtgagcccaaaggacttgattggagttgtgccgctggctatggtaagcaagccccgccctgggctgttagaactgaactcgggggaggggaaggcgccggcgccgcactgagtcccaggctgggtggggaaaaggggaggagccaagaatcaggacagt

SEQ ID NO: 22 - DNA, 259 bp, Artificial Sequence
  amplicon with primer sequence
gatggtgccaggaaagccactggaattgtcacacggcgagcacagagggccggccaccagtcctcgatgcttctgaaccctgaagcccgatgacatcttacgaggtggacgttggactgttcatgcgcatcgggtgtcagtgactcatggagaagaaatggggtaaatttttagtgatgttgctaatcattgaattctgttctctattaaattaagaaaatgttccaaaagccataagcctgaagattggccctgtgca

SEQ ID NO: 23 - DNA, 203 bp, Artificial Sequence
  amplicon with primer sequence
gcctgccgggaccctgggcccccgccgcctccgccaccacccccgccgcccccgccaccgcccggtctgtcccctcgggctcctgcgccgccacccgccggggccctcctcccggagcccggccagcgctgcgaggcggtcagcagcagccccccgccgcctccctgcgcccagaaccccctgcaccccagcccgtcccactc

SEQ ID NO: 24 - DNA, 265 bp, Artificial Sequence
  amplicon with primer sequence
agcccgaggggaagaacctggcccgtggggaggtgggggggaccgaaacggcgctgagccgagccgagagctacggggttcggagcagaggcagcggcagcggcagcggcagtaagagggaggggaggaggcaggagggcgcatggggcgccccggcccctccgacagcgcgccccctccggcccggccgcgctgaaagctccccagcgccgcgccttgaacccacgccccggggccatgccggtcatgaaggggttgctggccc

SEQ ID NO: 25 - DNA, 163 bp, homo sapiens
cgcgctgcgcagcccccgagccgttcgctacctggctgcggtcgcgcggccacctgtcctccgccctcggggcgccgcccagcttcgcgccaggcgccttctccagcgcccgccgccctttcccggcgagaccactcgggcgcgccccgcccgccggtccccg

SEQ ID NO: 26 - DNA, 204 bp, homo sapiens
ttcccctccggggctcctttccgccgagtccgcttcctgcagctgctgctagcaccgcagtccagggggagtgtcaaagaaggctgaaaaggaattgcaggagggtggagggaccaaaaggctacagagggcaaggtagggcggggatccctggtgcagacccgcagccccactggccctagggaaggagaaaccagattcccg

SEQ ID NO: 27 - DNA, 138 bp, homo sapiens
cgcccaacggctggacgcacaccccgccaggcctcctggaaacggtgccggtgctgcagagcccgcgaggtgtctgggagttgggcgagagctgcagacttggaggctcttatacctccgtgcaggcggaaagtttgg

SEQ ID NO: 28 - DNA, 218 bp, homo sapiens
cggactgtgaagcagtggtgtttcacgcttccatccccagaccatcaattattgacacgcccaaggtgagtgagtgttcgctctgcgattatagacgggatggagctggaggagcgttgggatcatgtggcgaatgtttcagcaaacaaactcatttaaccttactgaataaggcattgcgggcgttctcactagtgcgaagaagtgtgttaaagccg

SEQ ID NO: 29 - DNA, 194 bp, homo sapiens
tcacgtggcaggtcgagtaccccggagagatcacgtctgacttgggagtgtccaagatctatgtgagcccaaaggacttgattggagttgtgccgctggctatggtaagcaagccccgccctgggctgttagaactgaactcgggggaggggaaggcgccggcgccgcactgagtcccaggctgggtggggaaa

SEQ ID NO: 30 - DNA, 194 bp, homo sapiens
acacggcgagcacagagggccggccaccagtcctcgatgcttctgaaccctgaagcccgatgacatcttacgaggtggacgttggactgttcatgcgcatcgggtgtcagtgactcatggagaagaaatggggtaaatttttagtgatgttgctaatcattgaattctgttctctattaaattaagaaaatgtt

SEQ ID NO: 31 - DNA, 147 bp, homo sapiens
cgccgcctccgccaccacccccgccgcccccgccaccgcccggtctgtcccctcgggctcctgcgccgccacccgccggggccctcctcccggagcccggccagcgctgcgaggcggtcagcagcagccccccgccgcctccctgcg

SEQ ID NO: 32 - DNA, 221 bp, homo sapiens
ccgtggggaggtgggggggaccgaaacggcgctgagccgagccgagagctacggggttcggagcagaggcagcggcagcggcagcggcagtaagagggaggggaggaggcaggagggcgcatggggcgccccggcccctccgacagcgcgccccctccggcccggccgcgctgaaagctccccagcgccgcgccttgaacccacgccccggggccatgccg